SEQUENCE LISTING (4 sequences)

SEQ ID NO: 1 - DNA, 20 bp, Artificial Sequence
  chemically synthesized primer
gatctcaggaaacaggctcg

SEQ ID NO: 2 - DNA, 20 bp, Artificial Sequence
  chemically synthesized primer
cctgtaagccttgccaaaag

SEQ ID NO: 3 - DNA, 20 bp, Artificial Sequence
  chemically synthesized primer
gtgggccgctctaggcacca

SEQ ID NO: 4 - DNA, 20 bp, Artificial Sequence
  chemically synthesized primer
tggccttagggtgcaggggg

(19) United States
(12) Patent Application Publication
(10) Pub. No.: US 2005/0256179 A1
(43) Pub. Date: Nov. 17, 2005

(54) Selective pharmacologic inhibition of protein trafficking and related methods of treating human diseases

(76) Inventors: Jagadish C. Sircar, San Diego, CA (US); Mark L. Richards, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/915,722

(22) Filed: Aug. 9, 2004

Related U.S. Application Data
(60) Provisional application No. 60/493,497, filed on Aug. 8, 2003.

(51) Int. Cl. A61K 31/4184

(52) U.S. Cl. 514/394

(57) ABSTRACT

Preferred aspects of the present invention relate to the inhibition of intracellular protein trafficking pathways through selective pharmacologic down-regulation of specific resident ER and golgi proteins, and more particularly, to methods of treating a variety of disease conditions, which depend on these intracellular protein trafficking pathways.

### RELATED APPLICATIONS

This application claims priority under 35 U.S.C. §119(e) to U.S. Provisional Application No. 60/493,497 filed on Aug. 8, 2003.

### BACKGROUND OF THE INVENTION

1. Field of the Invention

Preferred aspects of the present invention relate to the inhibition of intracellular protein trafficking pathways through selective pharmacologic down-regulation of specific resident ER and golgi proteins, and more particularly, to methods of treating a variety of disease conditions, which depend on these intracellular protein trafficking pathways.

2. Description of the Related Art

In 1898, Camillio Golgi described a novel intracellular network which now bears his name (Golgi, 1898). The Golgi complex is an elaborate cytoplasmic organelle that has a prominent function in the processing, transporting, and sorting of intracellular proteins (reviewed in Gonatas, 1994; Mellman, 1995; Nilsson and Warren, 1994). Structurally, the Golgi complex is localized in the perinuclear region of most mammalian cells and is characterized by stacks of membrane-bound cisternae as well as a functionally distinct trans- (“TGN”), medial and cis-Golgi networks (“CGN”; see e.g., FIG. 1). It is proposed that the sorting functions of the Golgi complex are performed in TGN and CGN while the processing functions take place in the cis-, medial-, and trans-compartments (Mellman and Simons, 1992). The intracellular transport of newly synthesized proteins requires directed movement from the endoplasmic reticulum (“ER”), via transport vesicles to the cis-, medial- and trans-compartments of the Golgi complex, and in some cases, to the plasma membrane (Banfield et al., 1994; Farquhar and Palade, 1981; Griffiths et al., 1989; Mellman, 1995; Nilsson and Warren, 1994; Rothman and Orci, 1992).

Coatomer proteins COPI-coated vesicles are currently understood to mediate this anterograde transport across the intervening cistemae (Rothman, 1994; Schekman and Orci, 1996). Protein transport through the Golgi complex is mediated by small vesicles budding from a donor membrane and are targeted to, and fused with, an acceptor membrane (Rothman and Orci, 1992). Transport vesicles are known to move towards the TGN and are also hypothesized to move in the ‘retrograde’ direction to the CGN via the coat protein complex (coatomer proteins, e.g. beta-COPs, ref. (Banfield et al., 1994; Barlowe et al., 1994; Duden et al., 1991; Orci et al., 1997; Pelham, 1994; Seaman and Robinson, 1994; Serafini et al., 1991; Waters et al., 1991). In addition to protein trafficking, these pathways for the vesicular transport are believed to be important for the recycling of the membranous structures. The signals that control the vesicular traffic are poorly understood although it is known that intracellular microtubules are important components (Kreis, 1990; Mizuno and Singer, 1994). Other proteins of the Golgi complex believed to play a role include families of proteins such as the adaptins (Pearse and Robinson, 1990), GTP-binding (or “Rab”) proteins (Jena et al., 1994; Martinez et al., 1994; Nuoffer et al., 1994; Oka and Nakano, 1994; Pfeffer, 1994), ADP ribosylation factors (ARFs) (Steams et al., 1990), and resident enzymes (reviewed in (Farquhar, 1985; Nilsson and Warren, 1994). See also FIG. 26 illustrating proposed associations of various ER and Golgi proteins with distinct regions of the protein and membrane trafficking apparatus.

Recently, there has been a significant interest in Golgi apparatus disturbing agents, particularly Brefeldin A, due to its reported anti-tumor activity. Brefeldin A (BFA) was first described to be an antifungal, cytotoxic, and cancerostatic antibiotic (Haerri, et al. (1963) Chem. Abs.59:5726h). Brefeldin A was also reported to have anti-viral properties (Tamura et al. (1968) J. Antibiotics 21:161-166). In recent years, Brefeldin A has been studied extensively as a protein transport inhibitor. It is believed that Brefeldin A can reversibly disrupt the Golgi apparatus, thereby affecting protein transport through the cytoplasm (Domes et al. (1989) J. Cell Biol. 109:61-72 (1989); Lippincott-Schwartz et al. (1991) J. Cell Biol. 112:567-577). It is now known that Brefeldin A induces retrograde membrane transport from Golgi to the ER (Dinter et al.(1998) Histochem. Cell Biol. 109:571-590). Currently Brefeldin A is used as a tool by researchers to interfere with the processing and sorting of finished proteins in order to more fully understand protein trafficking. Because Brefeldin A broadly interferes with protein transport from the ER to the Golgi in most cells tested, it poses significant toxicity concerns and has not been developed as a therapeutic agent.

Accordingly, there is a need for elucidating ER and/or golgi proteins and mechanisms for modulating specific protein trafficking processes that are induced in various disease states, such as allergy, cancer and viral infection, and for identifying pharmacologic inibitors that selectively target such mechanisms.

### SUMMARY OF THE INVENTION

A method is disclosed in accordance with a preferred embodiment of the present invention for selectively inhibiting eukaryotic cell proliferation associated with a disease condition. The method comprises administering an amount of a composition sufficient to suppress expression of at least one ER/golgi resident protein associated with proliferation-dependent protein trafficking between the ER and golgi, such that the cell proliferation associated with the disease condition is inhibited. In preferred variations to the method, the at least one ER/golgi resident protein is selected from the group consisting of GS 15, GS28, nicastrin and a Rab. More preferably, the at least one ER/golgi resident protein is GS28.

In preferred embodiments of the method, the composition comprises a compound selected from the group consisting of: 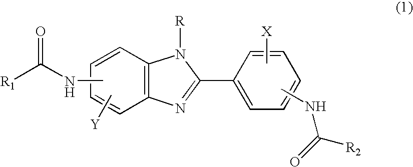
wherein X and Y are independently selected from the group consisting of H, alkyl, alkoxy, aryl, substituted aryl, hydroxy, halogen, amino, alkylamino, nitro, cyano, CF3, OCF3, CONH2, CONHR and NHCOR1;
wherein R is selected from the group consisting of H, CH₃, C₂H₅, C₃H₇, C₄H₉, CH₂Ph, and CH₂C₆H₄—F(p-); and
wherein R₁ and R₂ are independently selected from the group consisting of H, aryl, substituted aryl, cycloaryl substituted cycloaryl, multi-ring clycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like, wherein at least one of R1 and R2 are aromatic groups, 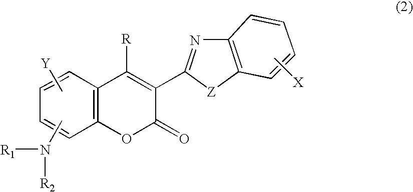
wherein X and Y are selected independently from the group consisting of alkyl, alkoxy, aryl, substituted aryl, heteroaryl, substituted heteroaryl, hydroxy, halogen, NO₂, CF₃, OCF3, NH₂, NHR₃, NR₃R₄ and CN;
wherein Z is selected from the group consisting of O, S, NH, and N—R′; wherein R′ is further selected from the group consisting of H, alkyl, aminoalkyl, and dialkylaminoalkyl;
wherein R is selected from the group consisting of H, alkyl, halogen, alkoxy, CF3 and OCF3; and
R1 and R2 are independently selected from the group consisting of H, alkyl, aminoalkyl, dialkylaminoalkyl, hydoxyalkyl, alkoxyalkyl, cycloalkyl, oxacycloalkyl and thiocycloalkyl, 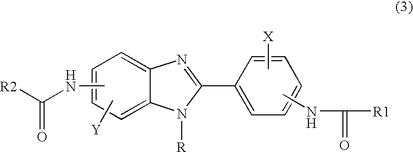
wherein X and Y are independently selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
wherein R is selected from the group consisting of H, CH3, C2H5, C3H7, C4H9, CH2Ph, and CH2C6H4-F(p-);
wherein R1 and R2 are independently selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, wherein said substitutions are not heterocyclic rings; and
wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, and substituted adamantyl are selected from the group consisting of alkyl, aryl, CF3, CH3, OCH3, OH, CN, COOR5, and COOH, 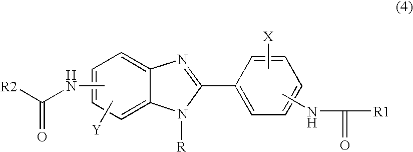
wherein X and Y are independently selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
wherein R is selected from the group consisting of H, CH3, C2H5, C3H7, C4H9, CH2Ph, and CH2C6H4-F(p-);
wherein R1 and R2 are independently selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, heterocyclic rings, and substituted heterocyclic rings;
wherein R1 and R2 cannot both be methyl groups;
wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, substituted adamantyl and substituted heterocyclic rings are selected from the group consisting of alkyl, acyl, aryl, CF3, CH3, OCH3, OH, CN, COOR5, COOH, COCF3, and heterocyclic rings; and
wherein at least one of R1, R2 or said substituents is a heterocyclic ring, 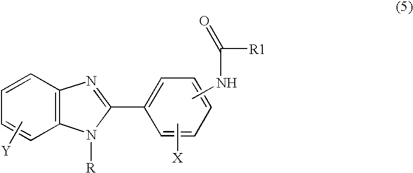
wherein X is selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
wherein R is selected from the group consisting of H, CH3, C2H5, C3H7, C4H9, CH2Ph, and CH2CH4-F(p-);
wherein Y is selected from the group consisting of mono, di, tri, and tetra substituted H, alkyl, alkoxy, aryl, benzo, substituted aryl, hydroxy, halogen, amino, alkylamino, nitro, cyano, CF3, OCF3, COPh, COOCH3, CONH2, CONHR, NHCONHR1, and NHCOR1;
wherein R1 is selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, heterocyclic rings containing one or more heteroatoms, and substituted heterocyclic rings; and
wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, substituted adamantyl, and substituted heterocyclic rings are selected from the group consisting of alkyl, aryl, CF3, CH3, OCH3, OH, CN, COOR, COOH, and heterocyclic rings, 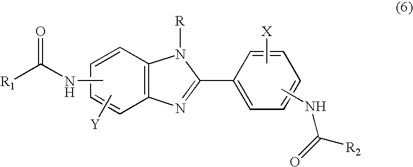
wherein X and Y are independently selected from the group consisting of H, alkyl, alkoxy, aryl, substituted aryl, hydroxy, halogen, amino, alkylamino, nitro, cyano, CF3, OCF3. CONH2, CONHR and NHCOR1;
wherein R is selected from the group consisting of H, CH3, C2H5, C3H7, C4H9, CH2Ph, CH2C6H4-F(p-); and
wherein R1 and R2 are independently selected from the group consisting of H, aryl, substituted aryl, cycloaryl substituted cycloaryl, multi-ring clycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like, wherein at least one of R1 and R2 are aromatic groups, 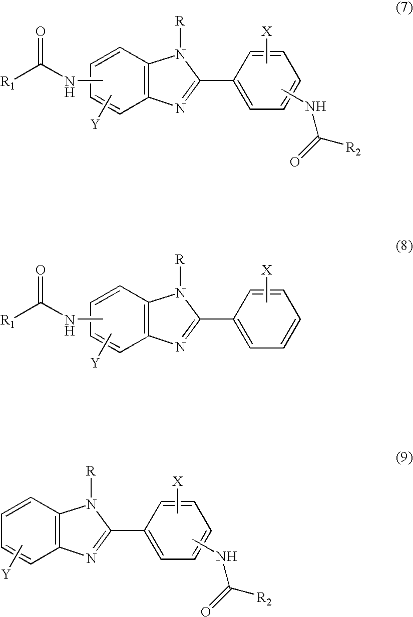
wherein X and Y are independently selected from the group consisting of H, alkyl, alkoxy, aryl, substituted aryl, hydroxy, halogen, amino, alkylamino, nitro, cyano, CF₃, OCF₃. CONH₂, CONHR and NHCOR₁;
wherein X and Y are independently selected from the group consisting of H, alkyl, alkoxy, aryl, substituted aryl, hydroxy, halogen, amino, alkylamino, nitro, cyano, CF3, OCF3. CONH2, CONHR and NHCOR1;
wherein R is selected from the group consisting of H, CH3, C2H5, C3H7, C4H9, CH2Ph, CH2C6H4-F(p-), COCH3, CO2CH2CH3, aminoalkyl and dialkylaminoalkyl; and
wherein R1 and R2 are independently selected from the group consisting of H, aryl, heteroaryl, thiophene, pyridyl, thiazolyl, isoxazolyl, oxazolyl, pyrimidinyl, substituted aryl, substituted heteroaryl, substituted thiophene, substituted pyridyl, substituted thiazolyl, substituted isoxazolyl, substituted oxazolyl, cycloaryl, cycloheteroaryl, quinolinyl, isoquinolinyl, substituted cycloaryl, substituted cycloheteroaryl, substituted quinolinyl, substituted isoqunolinyl, multi-ring cycloaryl, multi-ring cycloheteroaryl, benzyl, heteroaryl-methyl, substituted benzyl, substituted heteroaryl-methyl alkyl, dialkylaminoalkyl, cycloalkyl, cycloalkyl containing 1-3 heteroatoms, substituted cycloalkyl, substitute cycloalkyl containing 1-3 heteroatoms, multi-ring cycloalkyl, multiring cycloalkyl containing 1-3 heteroatoms, fused-ring aliphatic, fused-ring aliphatic containing 1-3 heteroatoms, cyclopropyl, substituted cyclopropyl, cyclobutyl, substituted cyclobutyl, cyclopentyl, pyrrole, piperidine, substituted cyclopentyl, cyclohexyl, substituted cyclohexyl, cycloheptyl, substituted cycloheptyl, bicycloheptyl, substituted pyrrole, substituted piperidine, bicyclooctyl, bicyclononyl, substituted bicycloalkenyl, adamantyl, and substituted adamantyl, heterocyclic ring, and substituted heterocyclic ring;
wherein at least one of R1 and R2 are aromatic groups or heteroaromatic groups; and
wherein R1 and R2 cannot both be phenyl groups, 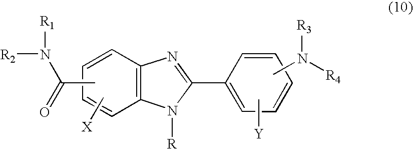
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, substituted polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein R3 and R4 are independently selected from the group consisting of H, alkyl, aryl, heteroaryl and COR′;
wherein R′ is selected from the group consisting of H, alkyl, substituted alkyl, C3-C9 cycloalkyl, substituted C3-C9 cycloalkyl, polycyclic aliphatics, substituted polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; wherein R′ is not haloalkyl;
wherein the substituent on R1, R2, and R′ is selected from the group consisting of H, halogens, polyhalogens, alkoxy group, substituted alkoxy, alkyl, substituted alkyl, dialkylaminoalkyl, hydroxyalkyl, carbonyl, OH, OCH3, COOH, OCOR′, COOR′, COR′, CN, CF3, OCF3, NO2, NR′R′, NHCOR′ and CONR′R′;
wherein X and Y are independently selected from the group consisting of H, halogens, alkoxy, substituted alkoxy, alkyl, substituted alkyl, dialkylaminoalkyl, hydroxyalkyl, OH, OCOR″, OCH3, COOH, CN, CF3, OCF3, NO2, COOR″, CHO and COR″;
wherein R″ is a C1-C8 alkyl, wherein said C1-C8 alkyl is selected from the group consisting of a straight chain, branched or cyclic alkyl; and wherein at least one of R1, R2, R3, or R4 is not H,

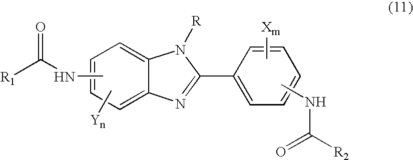

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF3, OCF3, COR1, COOR1, CONH2, CONHR1, and NHCOR1;
n is an integer from one to three;
m is an integer from one to four;
R is selected from the group consisting of H, CH3, C2H5, C3H7, C4H9, CH2Ph, CH2C6H4-F(p-), COCH3, COCH2CH3, CH2CH2N(CH3)2, and CH2CH2CH2N(CH3)2; and
R1 and R2 are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom, 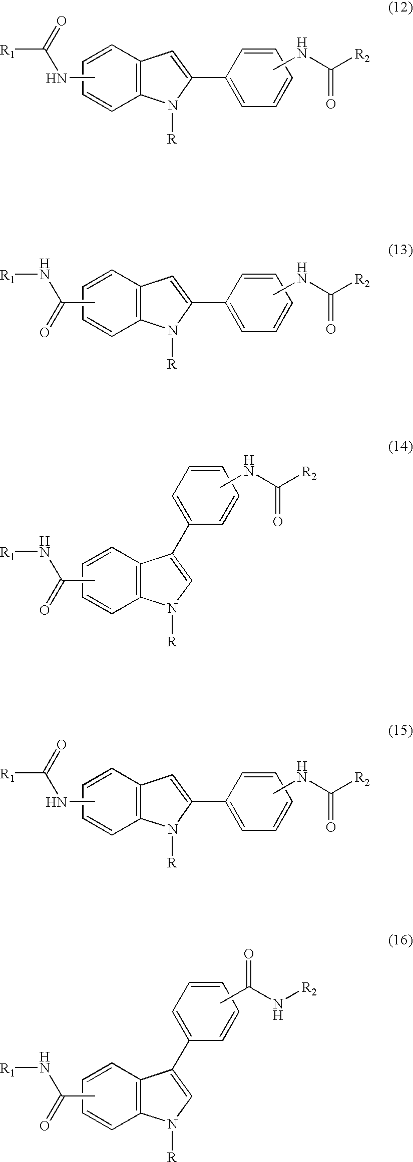 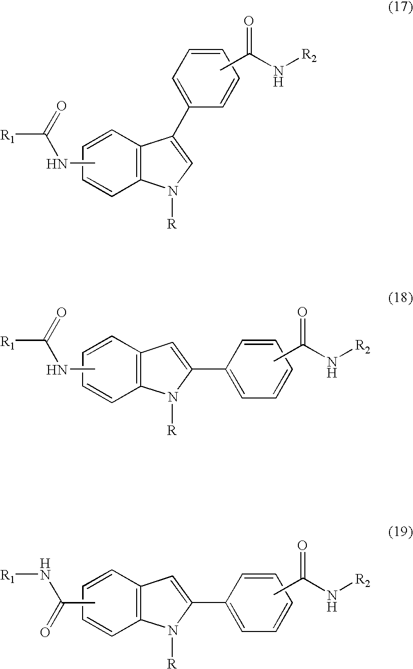
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is a C1-C8 alkyl, wherein said C1-C8 alkyl is selected from the group consisting of a straight chain, branched or cyclic alkyl, 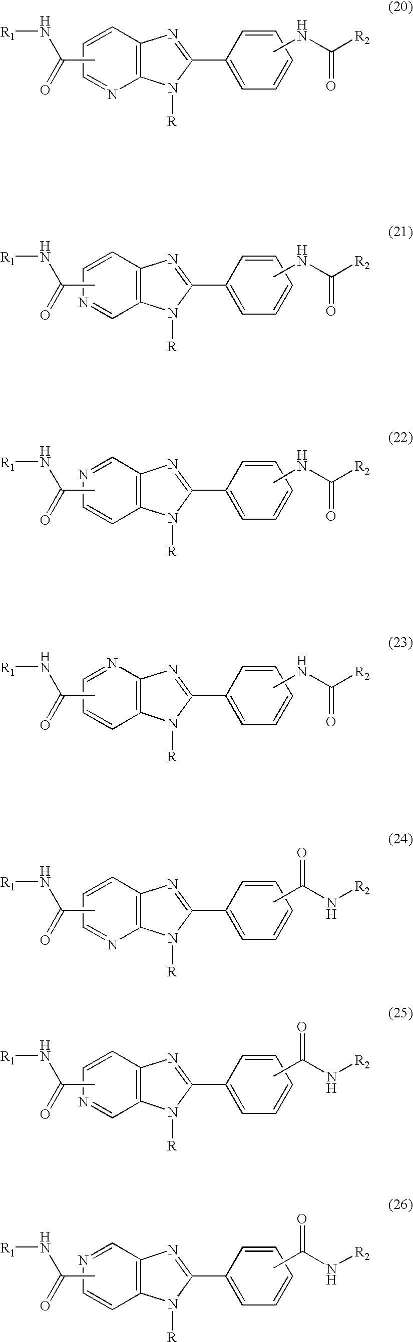 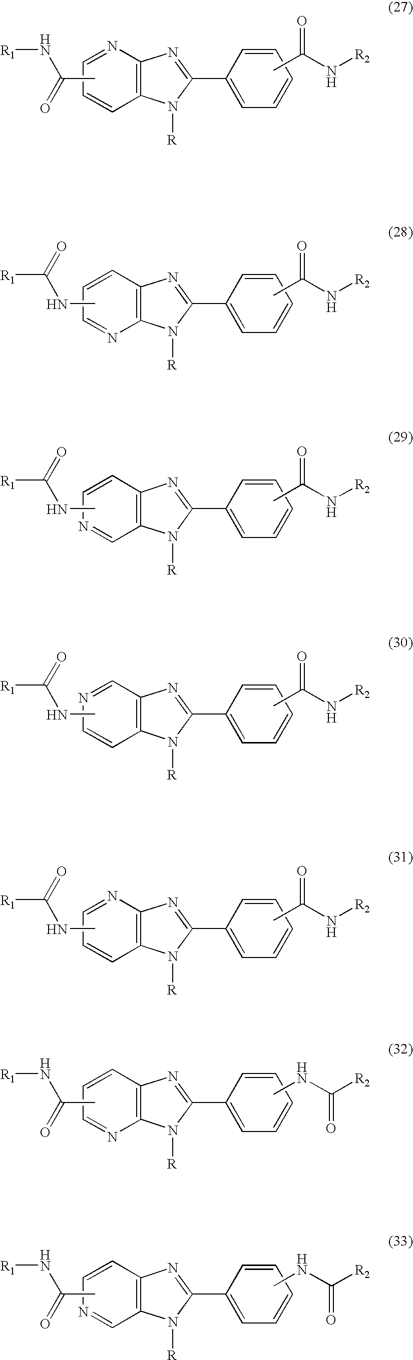 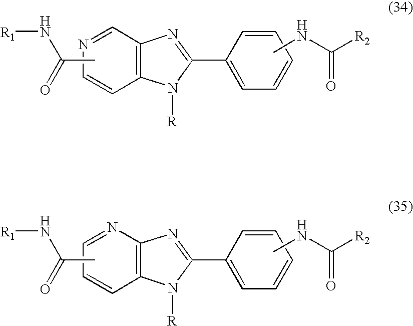
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is a C1-C8 alkyl, wherein said C1-C8 alkyl is selected from the group consisting of a straight chain, branched or cyclic alkyl, 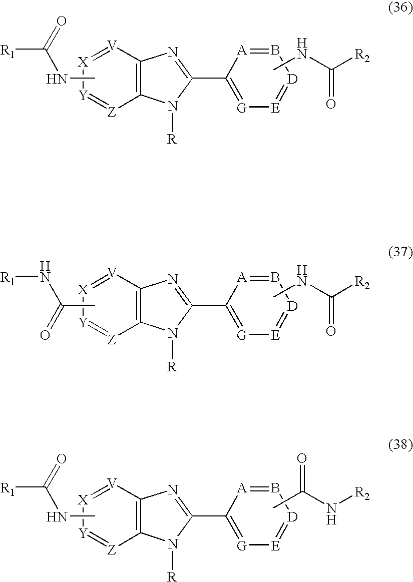
wherein A, B, D, E, G, V, X, Y, and Z are independently selected from carbon and nitrogen, with the proviso that at least one of A, B, D, E, G is nitrogen;
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′; and
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur, 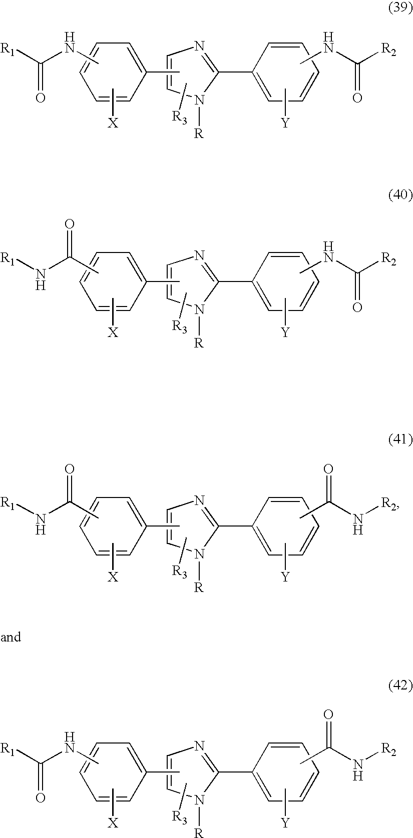
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R3, X, and Y are independently selected from the group consisting of H, halogen, alkoxy, substituted alkoxy, alkyl, substituted alkyl, dialkylaminoalkyl, hydroxyalkyl, OH, OCH3, COOH, CN, CF3, OCF3, NO2, COOR″, CHO, and COR″;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C1-C9 alkyl, wherein said C1-C9 alkyl is selected from the group consisting of straight chain alkyl, branched alkyl, and cyclic alkyl.

In more preferred embodiments of the method, the composition comprises the compound AVP 893.

In a variation to the method for selectively inhibiting eukaryotic cell proliferation associated with a disease condition, the composition comprises the compound: 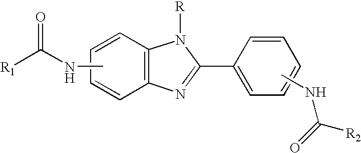
wherein R is selected from the group consisting of H, CH₃, C₂H₅, C₃H₇, C₄H₉, CH₂Ph, and CH₂C₆H₄—F(p-); and
wherein R₁ and R₂ are independently selected from the group consisting of H, aryl, substituted aryl, cycloaryl substituted cycloaryl, multi-ring cycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like; and
wherein said amount is sufficient to suppress expression of at least one ER/golgi resident protein involved in proliferation-dependent protein trafficking between the ER and golgi, such that the cell proliferation associated with the disease condition is inhibited.

In another variation to the method for selectively inhibiting eukaryotic cell proliferation, the composition comprises the compound: 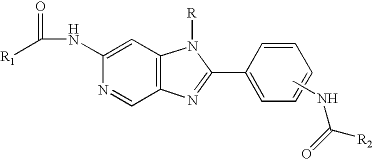
wherein R is selected from the group consisting of H, CH₃, C₂H₅, C₃H₇, C₄H₉, CH₂Ph, and CH₂C₆H₄—F(p-); and
wherein R₁ and R₂ are independently selected from the group consisting of H, aryl, substituted aryl, cycloaryl substituted cycloaryl, multi-ring cycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like; and
wherein said amount is sufficient to suppress expression of at least one ER/golgi resident protein involved in proliferation-dependent protein trafficking between the ER and golgi, such that the cell proliferation associated with the disease condition is inhibited.

In accordance with another preferred embodiment of the present invention, a method is disclosed for selectively inhibiting cytokine responses associated with a disease condition, comprising administering an amount of a composition sufficient to suppress expression of at least one ER/golgi resident protein involved in cytokine-dependent protein trafficking between the ER and golgi, such that the cytokine responses associated with the disease condition are inhibited. In preferred variations, the composition comprises a compound selected from the group consisting of compounds (1) through (42).

In accordance with another preferred embodiment of the present invention, a method is disclosed for selectively inhibiting viral replication, comprising administering an amount of a composition sufficient to suppress expression of at least one ER/golgi resident protein involved in viral protein trafficking between the ER and golgi, such that viral replication is inhibited. In preferred variations, the composition comprises a compound selected from the group consisting of compounds (1) through (42).

In accordance with another preferred embodiment of the present invention, a method is disclosed for selectively reducing B-cell secretion of IgE associated with an allergic reaction, comprising administering an amount of a composition sufficient to suppress expression of at least one ER/golgi resident protein involved in protein trafficking, such that the B-cell secretion of IgE is reduced. In preferred variations, the composition comprises a compound selected from the group consisting of compounds (1) through (42).

In accordance with another preferred embodiment of the present invention, a method is disclosed for diminishing GS28-mediated protein trafficking, comprising administering an amount of a composition sufficient to suppress GS28 expression such that GS28-mediated protein trafficking is diminished. In preferred variations, the composition comprises a compound selected from the group consisting of compounds (1) through (42).

In accordance with another preferred embodiment of the present invention, a method is disclosed for modifying effects of external influences on eukaryotic cells, wherein said external influences depend on GS28-mediated protein trafficking, the method comprising administering an amount of a composition sufficient to alter GS28 expression in the cells such that the external influences are modified. In preferred variations, the composition comprises a compound selected from the group consisting of compounds (1) through (42).

In accordance with another preferred embodiment of the present invention, a method is disclosed for treating a viral infection, comprising administering an amount of a composition sufficient to reduce GS28 expression and thereby reduce progeny virion assembly, such that the viral infection is treated. In preferred variations, the composition comprises a compound selected from the group consisting of compounds (1) through (42).

In accordance with another preferred embodiment of the present invention, a method is disclosed for treating cancer, comprising administering an amount of an agent sufficient to inhibit expression of at least one ER-golgi protein, wherein said at least one ER-golgi protein is required for cancer cell proliferation. In preferred variations, the composition comprises a compound selected from the group consisting of compounds (1) through (42).

### BRIEF DESCRIPTION OF THE DRAWINGS

FIG. 1 is a schematic illustrating intracellular protein trafficking.

FIG. 2 shows the IgE response to antigen ex vivo.

FIG. 3 shows the IgE response to IL-4+αCD40 Ab in human PBL in vitro.

FIG. 4 illustrates murine spleen T cell cytokine responses in vitro.

FIG. 5 shows human PBL T cell cytokine responses.

FIG. 6 show CD23 on human monocytes.

FIG. 7 shows spleen cell proliferation response to AVP 893.

FIG. 8 shows proliferation of human PBL in response to stimulus and drug in vitro.

FIG. 9 shows an NCI 60-cell panel.

FIG. 10 is a schematic of a BAL protocol #1 and illustrates the cells in BAL wash.

FIG. 11 shows the AHR response in vivo.

FIG. 12 shows the effect of AVP 25752 on B16-F1 mouse melanoma tumor growth.

FIG. 13 shows the effect of AVP 893 on HS294t human melanoma tumor growth.

FIG. 14 is a dose response of AVP 13358 on various biochemical assays.

FIG. 15 is a kinase screen of AVP 13358.

FIG. 16 shows the PowerBlot results of the effect of AVP 893 on protein expression.

FIG. 17 shows the time course of AVP 893 action in B16 cells.

FIG. 18 shows the effect of AVP 893 on nicastrin and GS28 expression in various cells at 16 hours.

FIG. 19 shows the effect of AVP 893 on nicastrin, calnexin and GS28 expression in various cells overnight.

FIG. 20 shows the effect of AVP 893 on nicastrin, n-gly, calnexin and GS28 expression in various cells overnight.

FIG. 21 shows inhibition of stimulated protein expression in BALB/c spleen cells by AVP 893.

FIG. 22 shows dose-responsive inhibition of PMA/ionomycin-stimulated nicastrin and GS28 expression in BALB/c spleen cells by various compounds.

FIG. 23 shows the PMA effect on AVP 893 inhibition of PBL proliferation response to IL-4/αCD40 Ab.

FIG. 24 shows the selective dose-response of AVP 893 in down-regulating IL-4/αCD40 Ab induced protein expression after 48 hours in the presence and absence of PMA.

FIG. 25 shows GS28 mRNA response to AVP 893 in human PBL.

FIG. 26 is a schematic showing involvement of various ER and golgi proteins in protein trafficking pathways.

FIG. 27 shows dose-responsive inhibition by AVP 893 of Rab expression in 18-20 hour cultures.

FIG. 28 shows a comparison of the effects of AVP 893 on GS28 and Rab1a protein expression in 3T3 cells.

FIG. 29 shows the effect of AVP 893 on expression of resident golgi proteins.

FIG. 30 shows the effect of AVP 893 on Mannosidase II expression.

FIG. 31 shows the effect of AVP 893 on Rab1B expression in Vero cells.

FIG. 32 shows the effect of AVP 893 on golgi morphology in MOLT4 cells.

FIG. 33 shows the effect of AVP 893 on protein expression in B16 cells.

FIG. 34 shows the Rab6 distribution in B16 cells.

FIG. 35 shows the Rab1B distribution in B16 cells.

FIG. 36 shows the SNAP23 response to AVP 893 in B16 cells.

FIG. 37 shows NCI results with AVP 893 and Brefeldin A.

FIG. 38 shows the effects of AVP 893 and Brefeldin A on GS28 and nicastrin expression.

FIG. 39 shows the Rab6 response to Brefeldin A and AVP 893 in 3T3 cells.

FIG. 40 shows a quantitative comparison of GS28 and nicastrin in 6 cell lines.

FIG. 41 shows unique activity of AVP 893 on resident golgi proteins compared to known pharmacological agents in 3T3 cells.

FIG. 42 shows the differential effects of AVP 893 and Brefeldin A on GS28, Calnexin and Rab6 expression.

FIG. 43 shows the differential effects of AVP 893, Brefeldin A and Nocodozole on Mannosidase II expression.

FIG. 44 shows the effect of AVP 893 on HSV-2 propagation in Vero cells in vitro.

FIG. 45 showing action of the AVP 893 on gE expression in HSV-2 infected Vero cells.

FIG. 46 is a schematic showing the elucidated mechanism of action of the AVP compounds.

FIG. 47 is a schematic showing the multiple effects of the selective inhibition of GS28 protein expression by AVP 893.

### DETAILED DESCRIPTION OF THE PREFERRED EMBODIMENT

An effort to develop novel therapeutic agents to treat allergic disorders led to the identification of lead compounds that suppress IgE responses ex vivo, in vitro, and in vivo. Additional series of compounds have been subsequently synthesized based upon their activity in suppressing IgE responses in vitro. These series of compounds, as well as their synthetic pathways and their biological activities, are detailed in issued U.S. Pat. Nos. 6,271,390, 6,451,829, 6,369,091, 6,303,645, and 6,759,425, and co-pending U.S. patent application Ser. Nos. 09/983,054, 10/103,258, 10/661,139, 10/661,296 and 10/821,667, and co-pending international Patent Application Nos. PCT/US03/05985 and PCT/US03/06981; all of which are incorporated herein in their entirety by reference thereto. These compounds have been discovered to have other biological effects in addition to suppression of IgE, including inhibition of cytokine production/release, suppression of cell surface receptor expression, and inhibition of cellular proliferation. Some of the lead compounds included in this series are AVP 893, AVP 13358, and AVP 25752, all of which share the above-described biological effects while the activity of a number of other analogs have been defined on the basis of one or more of these actions.

The compounds were not identified on the basis of a target-based assay but rather based on their cellular activity. Thus, the mechanism of action has until recently been a mystery. The activity profile of these compounds is highly unusual and suggests that their shared mechanism of action is novel. These agents do not affect the activity of more than 70 kinases and other enzymes. Moreover, a screen of drug activity on the expression of over 950 proteins revealed only a handful of modulated proteins in vitro. These results and the studies subsequent to this form the basis of the patent application described herein.

Several distinct series of chemical compounds are described that have in common a suppressive action on the expression of IgE, elicitation of cytokines, expression of membrane receptors, and cellular proliferation. These series include the following compounds: 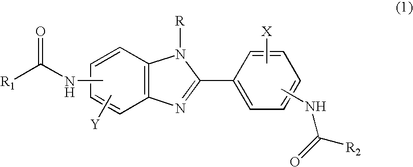
wherein X and Y are independently selected from the group consisting of H, alkyl, alkoxy, aryl, substituted aryl, hydroxy, halogen, amino, alkylamino, nitro, cyano, CF₃, OCF₃, CONH₂, CONHR and NHCOR₁;
wherein R is selected from the group consisting of H, CH₃, C₂H₅, C₃H₇, C₄H₉, CH₂Ph, and CH₂C₆H₄—F(p-); and
wherein R₁ and R₂ are independently selected from the group consisting of H, aryl, substituted aryl, cycloaryl substituted cycloaryl, multi-ring clycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like, wherein at least one of R1 and R2 are aromatic groups, 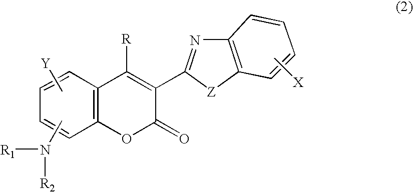
wherein X and Y are selected independently from the group consisting of alkyl, alkoxy, aryl, substituted aryl, heteroaryl, substituted heteroaryl, hydroxy, halogen, NO₂, CF₃, OCF₃, NH₂, NHR₃, NR₃R₄ and CN;
wherein Z is selected from the group consisting of O, S, NH, and N—R′; wherein R′ is further selected from the group consisting of H, alkyl, aminoalkyl, and dialkylaminoalkyl;
wherein R is selected from the group consisting of H, alkyl, halogen, alkoxy, CF3 and OCF3; and
R1 and R2 are independently selected from the group consisting of H, alkyl, aminoalkyl, dialkylaminoalkyl, hydoxyalkyl, alkoxyalkyl, cycloalkyl, oxacycloalkyl and thiocycloalkyl, 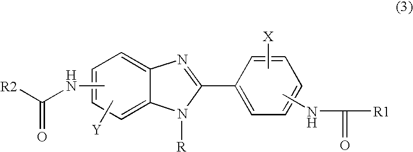
   wherein X and Y are independently selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
   wherein R is selected from the group consisting of H, CH3, C2H5, C3H7, C4H9, CH2Ph, and CH2C6H4-F(p-);
   wherein R1 and R2 are independently selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, wherein said substitutions are not heterocyclic rings; and
   wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, and substituted adamantyl are selected from the group consisting of alkyl, aryl, CF3, CH3, OCH3, OH, CN, COOR5, and COOH, R (4) wherein X and Y are independently selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1; 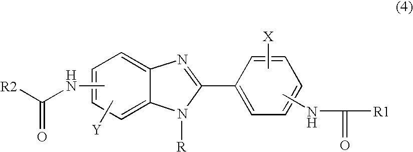
   wherein R is selected from the group consisting of H, CH3, C2H5, C3H7, C4H9, CH2Ph, and CH2C6H4-F(p-);
   wherein R1 and R2 are independently selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, heterocyclic rings, and substituted heterocyclic rings;
   wherein R1 and R2 cannot both be methyl groups;
   wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, substituted adamantyl and substituted heterocyclic rings are selected from the group consisting of alkyl, acyl, aryl, CF3, CH3, OCH3, OH, CN, COOR5, COOH, COCF3, and heterocyclic rings; and
   wherein at least one of R1, R2 or said substituents is a heterocyclic ring, 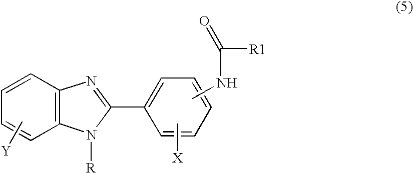
   wherein X is selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
   wherein R is selected from the group consisting of H, CH3, C2H5, C3H7, C4H9, CH2Ph, and CH2CH4-F(p-);
   wherein Y is selected from the group consisting of mono, di, tri, and tetra substituted H, alkyl, alkoxy, aryl, benzo, substituted aryl, hydroxy, halogen, amino, alkylamino, nitro, cyano, CF3, OCF3, COPh, COOCH3, CONH2, CONHR, NHCONHR1, and NHCOR1;
   wherein R1 is selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, heterocyclic rings containing one or more heteroatoms, and substituted heterocyclic rings; and
   wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, substituted adamantyl, and substituted heterocyclic rings are selected from the group consisting of alkyl, aryl, CF3, CH3, OCH3, OH, CN, COOR, COOH, and heterocyclic rings, 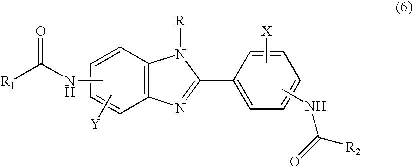
   wherein X and Y are independently selected from the group consisting of H, alkyl, alkoxy, aryl, substituted aryl, hydroxy, halogen, amino, alkylamino, nitro, cyano, CF3, OCF3. CONH2, CONHR and NHCOR1;
   wherein R is selected from the group consisting of H, CH3, C2H5, C3H7, C4H9, CH2Ph, CH2C6H4-F(p-); and
   wherein R1 and R2 are independently selected from the group consisting of H, aryl, substituted aryl, cycloaryl substituted cycloaryl, multi-ring clycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like, wherein at least one of R1 and R2 are aromatic groups, 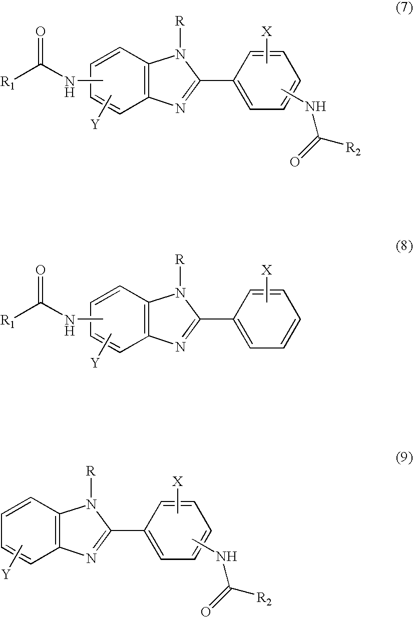
   wherein X and Y are independently selected from the group consisting of H, alkyl, alkoxy, aryl, substituted aryl, hydroxy, halogen, amino, alkylamino, nitro, cyano, CF₃, OCF₃. CONH₂, CONHR and NHCOR₁;
   wherein X and Y are independently selected from the group consisting of H, alkyl, alkoxy, aryl, substituted aryl, hydroxy, halogen, amino, alkylamino, nitro, cyano, CF3, OCF3. CONH2, CONHR and NHCOR1;
   wherein R is selected from the group consisting of H, CH3, C2H5, C3H7, C4H9, CH2Ph, CH2C6H4-F(p-), COCH3, CO2CH2CH3, aminoalkyl and dialkylaminoalkyl; and
   wherein R1 and R2 are independently selected from the group consisting of H, aryl, heteroaryl, thiophene, pyridyl, thiazolyl, isoxazolyl, oxazolyl, pyrimidinyl, substituted aryl, substituted heteroaryl, substituted thiophene, substituted pyridyl, substituted thiazolyl, substituted isoxazolyl, substituted oxazolyl, cycloaryl, cycloheteroaryl, quinolinyl, isoquinolinyl, substituted cycloaryl, substituted cycloheteroaryl, substituted quinolinyl, substituted isoqunolinyl, multi-ring cycloaryl, multi-ring cycloheteroaryl, benzyl, heteroaryl-methyl, substituted benzyl, substituted heteroaryl-methyl alkyl, dialkylaminoalkyl, cycloalkyl, cycloalkyl containing 1-3 heteroatoms, substituted cycloalkyl, substitute cycloalkyl containing 1-3 heteroatoms, multi-ring cycloalkyl, multiring cycloalkyl containing 1-3 heteroatoms, fused-ring aliphatic, fused-ring aliphatic containing 1-3 heteroatoms, cyclopropyl, substituted cyclopropyl, cyclobutyl, substituted cyclobutyl, cyclopentyl, pyrrole, piperidine, substituted cyclopentyl, cyclohexyl, substituted cyclohexyl, cycloheptyl, substituted cycloheptyl, bicycloheptyl, substituted pyrrole, substituted piperidine, bicyclooctyl, bicyclononyl, substituted bicycloalkenyl, adamantyl, and substituted adamantyl, heterocyclic ring, and substituted heterocyclic ring;
   wherein at least one of R1 and R2 are aromatic groups or heteroaromatic groups; and
   wherein R1 and R2 cannot both be phenyl groups, 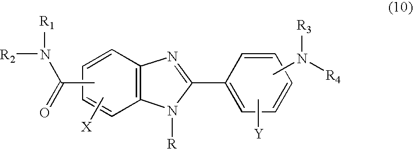
   wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
   wherein R1 and R2 are independently selected from the group consisting of H, alkyl, substituted alkyl, C3-C9 cycloalkyl, substituted C3-C9 cycloalkyl, polycyclic aliphatic groups, substituted polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
   wherein R3 and R4 are independently selected from the group consisting of H, alkyl, aryl, heteroaryl and COR′;
   wherein R′ is selected from the group consisting of H, alkyl, substituted alkyl, C3-C9 cycloalkyl, substituted C3-C9 cycloalkyl, polycyclic aliphatics, substituted polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; wherein R′ is not haloalkyl;
   wherein the substituent on R1, R2, and R′ is selected from the group consisting of H, halogens, polyhalogens, alkoxy group, substituted alkoxy, alkyl, substituted alkyl, dialkylaminoalkyl, hydroxyalkyl, carbonyl, OH, OCH3, COOH, OCOR′, COOR′, COR′, CN, CF3, OCF3, NO2, NR′R′, NHCOR′ and CONR′R′;
   wherein X and Y are independently selected from the group consisting of H, halogens, alkoxy, substituted alkoxy, alkyl, substituted alkyl, dialkylaminoalkyl, hydroxyalkyl, OH, OCOR″, OCH3, COOH, CN, CF3, OCF3, NO2, COOR″, CHO and COR″;
   wherein R″ is a C1-C8 alkyl, wherein said C1-C8 alkyl is selected from the group consisting of a straight chain, branched or cyclic alkyl; and wherein at least one of R1, R2, R3, or R4 is not H,

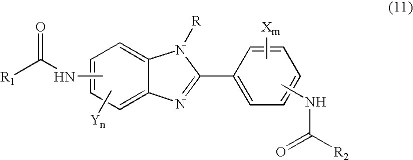

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF3, OCF3, COR1, COOR1, CONH2, CONHR1, and NHCOR1;
n is an integer from one to three;
m is an integer from one to four;
R is selected from the group consisting of H, CH3, C2H5, C3H7, C4H9, CH2Ph, CH2C6H4-F(p-), COCH3, COCH2CH3, CH2CH2N(CH3)2, and CH2CH2CH2N(CH3)2; and
R1 and R2 are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom, 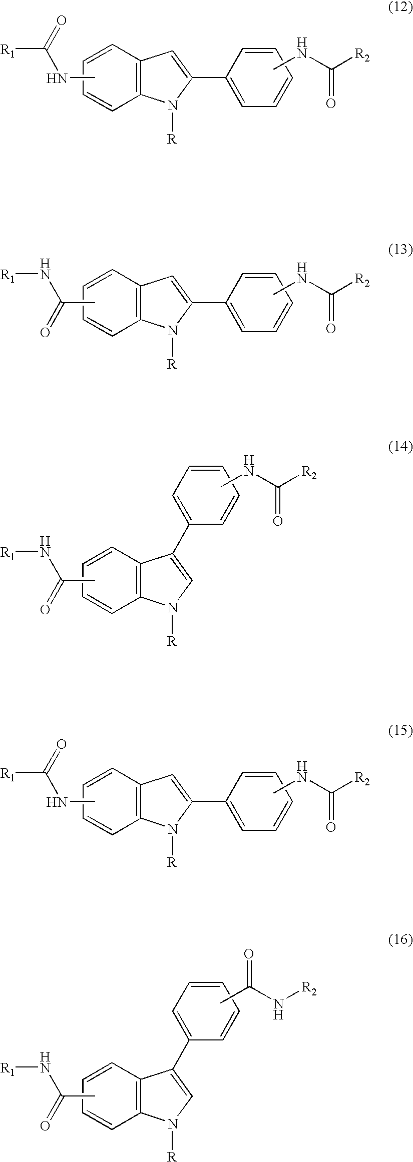 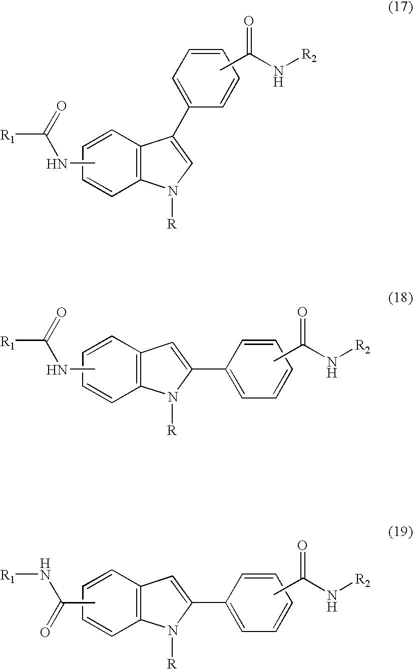
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and wherein R″ is a C1-C8 alkyl, wherein said C1-C8 alkyl is selected from the group consisting of a straight chain, branched or cyclic alkyl, 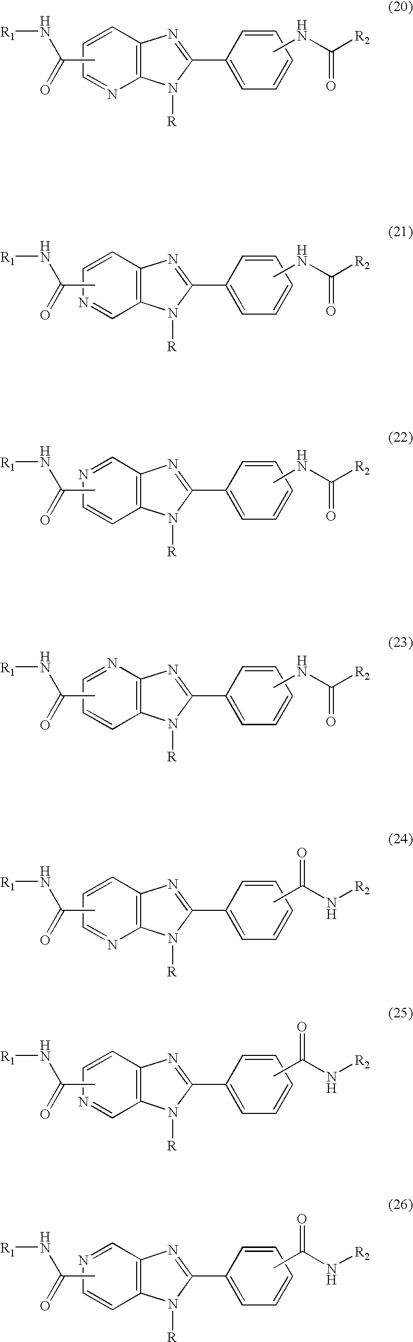 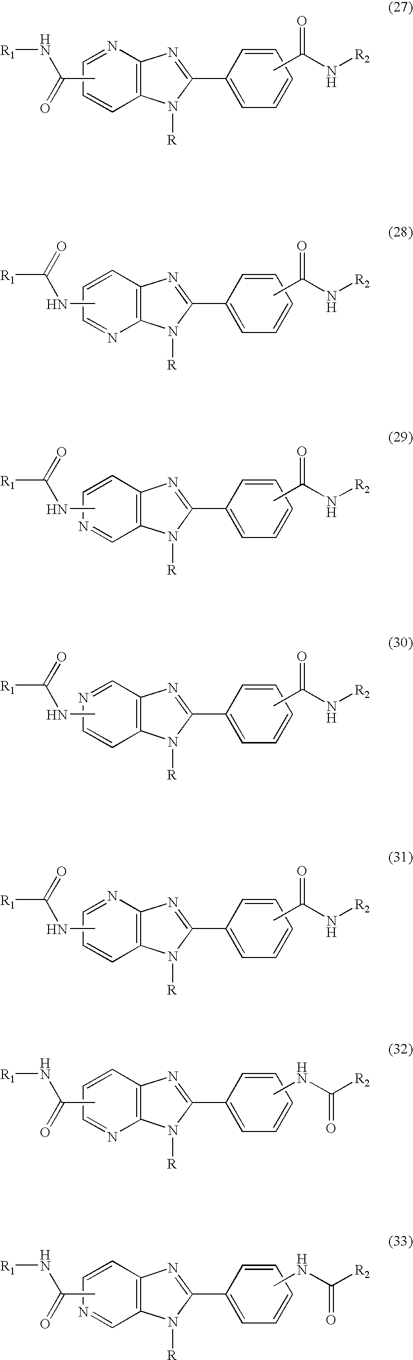 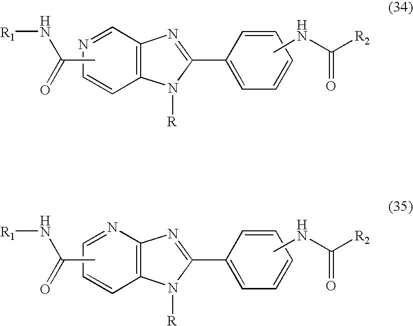
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is a C1-C8 alkyl, wherein said C1-C8 alkyl is selected from the group consisting of a straight chain, branched or cyclic alkyl, 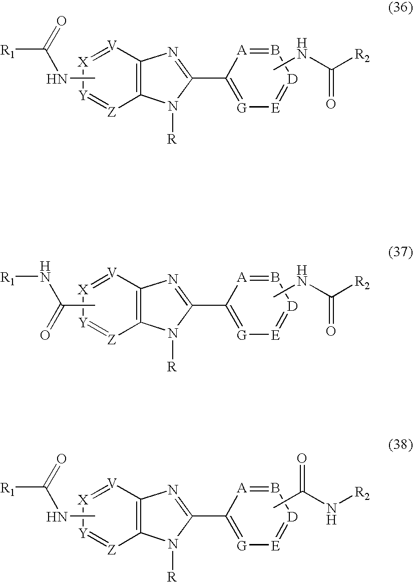
wherein A, B, D, E, G, V, X, Y, and Z are independently selected from carbon and nitrogen, with the proviso that at least one of A, B, D, E, G is nitrogen;
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′; and
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur, 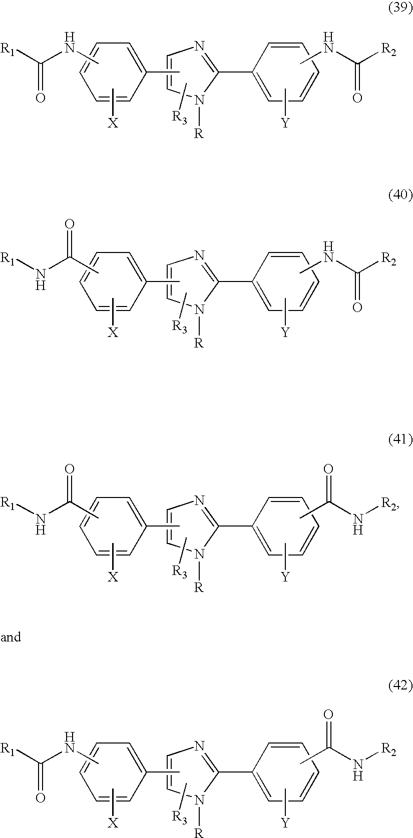
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R3, X, and Y are independently selected from the group consisting of H, halogen, alkoxy, substituted alkoxy, alkyl, substituted alkyl, dialkylaminoalkyl, hydroxyalkyl, OH, OCH3, COOH, CN, CF3, OCF3, NO2, COOR″, CHO, and COR″;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected form the group consisting of nitrogen, oxygen and sulfur; and
wherein R″is selected from the group consisting of C1-C9 alkyl, wherein said C1-C9 alkyl is selected from the group consisting of straight chain alkyl, branched alkyl, and cyclic alkyl.

Numerous specific compounds that exemplify the generic formulas (1) through (42) have been synthesized and tested in accordance with preferred aspects of the present invention. Some preferred compounds are listed below in TABLE 1.

TABLE 1

| AVP NUMBER | STRUCTURE |
|---|---|
| 13358 | 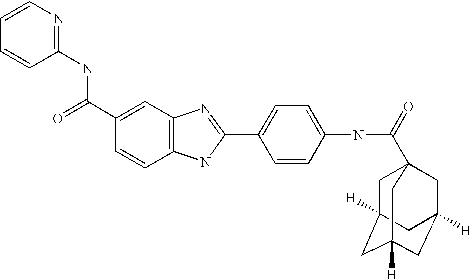 |
| 26135 | 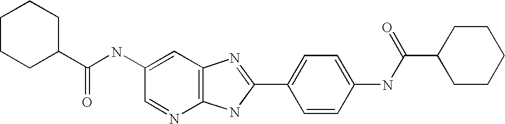 |
| 26294 | 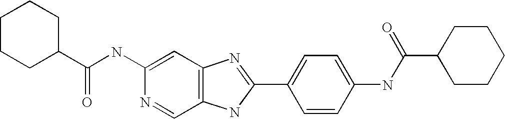 |
| 26296 | 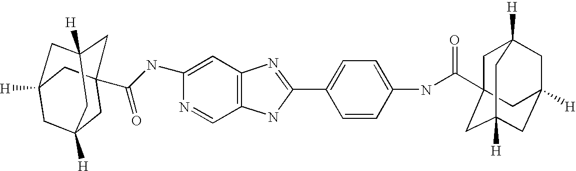 |
| 26350 | 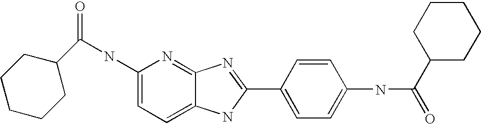 |
| 26359 | 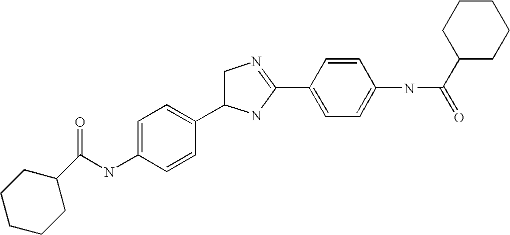 |
| 26405 | 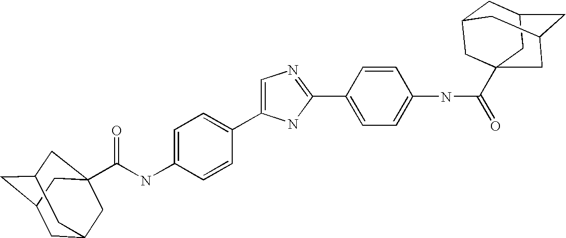 |
| 26410 | 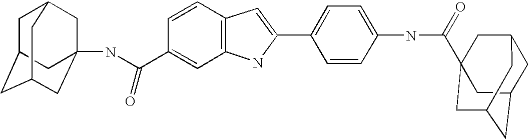 |
| 26411 | 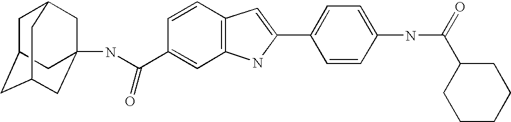 |
| 26412 | 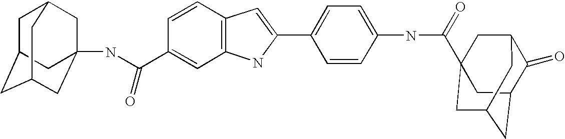 |
| 26428 | 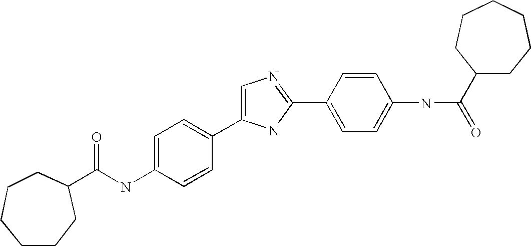 |
| 26438 | 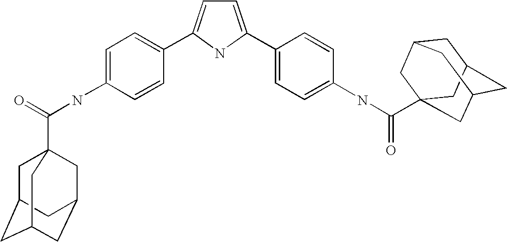 |
| 26449 | 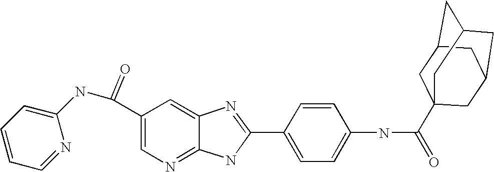 |
| 26465 | 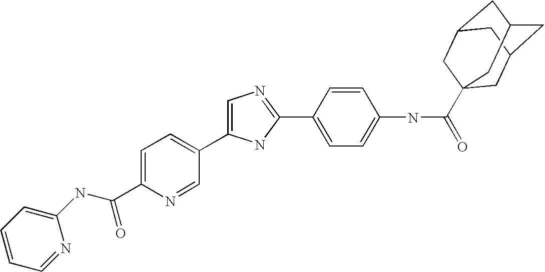 |
| 26472 | 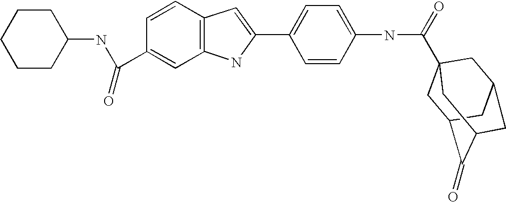 |
| 26489 | 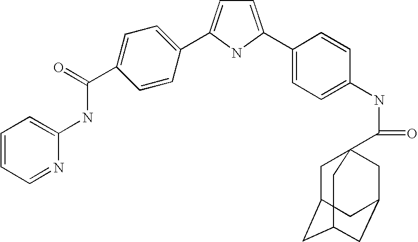 |
| 893 | 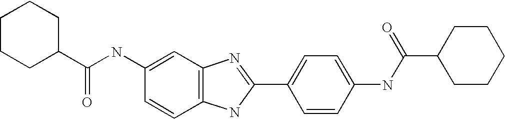 |

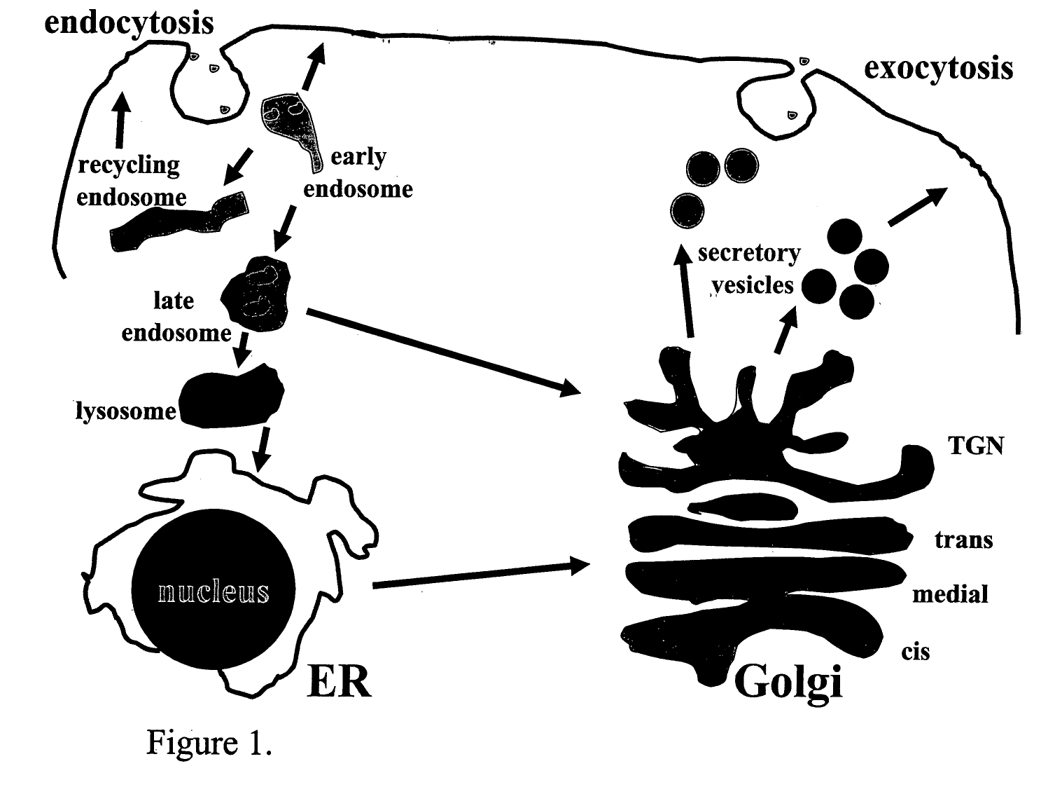

Recently, the mechanism of action of these compounds was investigated in order to link the diverse actions of these compounds. These studies led to the revelation that intracellular protein trafficking (FIG. 1) is affected by drug treatment in vitro. This novel mechanism of action has no known duplication by drugs utilized in the treatment of human disease. Moreover, only a handful of chemicals used in the dissection of molecular mechanisms of cellular processes are known to inhibit intracellular protein trafficking. The compounds described herein affect the expression of particular proteins responsible for movement of cellular proteins between the endoplasmic reticulum (ER) and the golgi in all primary cells and many tumor cell lines. Moreover, studies designed to track intracellular protein movement show that the compounds block the ER-to-golgi movement of proteins in vitro by a mechanism that is distinct from that utilized by other known inhibitors such as Monensin and Brefeldin A. The described activity explains the known diverse actions of the AVP compounds and successfully predicts additional activity, particularly inhibition of viral propagation.

Assays

In one preferred embodiment, the present invention is directed to small molecule inhibitors of IgE (synthesis and/or release) which are useful in the treatment of allergy and/or asthma or any diseases where IgE is pathogenic. The particular compounds disclosed herein were identified by their ability to suppress IgE levels in both ex vivo and in vivo assays. Development and optimization of clinical treatment regimens can be monitored by those of skill in the art by reference to the ex vivo and in vivo assays described below.

Ex Vivo Assay—This system begins with in vivo antigen priming and measures secondary antibody responses in vitro. The basic protocol was documented and optimized for a range of parameters including: antigen dose for priming and time span following priming, number of cells cultured in vitro, antigen concentrations for eliciting secondary IgE (and other Ig's) response in vitro, fetal bovine serum (FBS) batch that will permit optimal IgE response in vitro, the importance of primed CD4+ T cells and hapten-specific B cells, and specificity of the ELISA assay for IgE (Marcelletti and Katz, Cellular Immunology 135:471-489 (1991); incorporated herein by reference).

The actual protocol utilized for this project was adapted for a more high throughput analyses. BALB/cByj mice were immunized i.p. with 10 μg DNP-KLH adsorbed onto 4 mg alum and sacrificed after 15 days. Spleens were excised and homogenized in a tissue grinder, washed twice, and maintained in DMEM supplemented with 10% FBS, 100 U/ml penicillin, 100 μg/ml streptomycin and 0.0005% 2-mercaptoethanol. Spleen cell cultures were established (2-3 million cells/ml, 0.2 ml/well in quadruplicate, 96-well plates) in the presence or absence of DNP-KLH (10 ng/ml). Test compounds (2 μg/ml and 50 ng/ml) were added to the spleen cell cultures containing antigen and incubated at 37° C. for 8 days in an atmosphere of 10% CO2.

Culture supernatants were collected after 8 days and Ig's were measured by a modification of the specific isotype-selective ELISA assay described by Marcelletti and Katz (Supra). The assay was modified to facilitate high throughput. ELISA plates were prepared by coating with DNP-KLH overnight. After blocking with bovine serum albumin (BSA), an aliquot of each culture supernatant was diluted (1:4 in phosphate buffered saline (PBS) with BSA, sodium azide and Tween 20), added to the ELISA plates, and incubated overnight in a humidified box at 4° C. IgE levels were quantitated following successive incubations with biotinylated-goat antimouse IgE (b-GAME), AP-streptavidin and substrate.

Antigen-specific IgG1 was measured similarly, except that culture supernatants were diluted 200-fold and biotinylated-goat antimouse IgG1 (b-GAMG1) was substituted for b-GAME. IgG2a was measured in ELISA plates that were coated with DNP-KLH following a 1:20 dilution of culture supernatants and incubation with biotinylated-goat antimouse IgG2a (b-GAMG2a). Quantitation of each isotype was determined by comparison to a standard curve. The level of detectability of all antibody was about 200-400 μg/ml and there was less than 0.001% cross-reactivity with any other Ig isotype in the ELISA for IgE.

In Vivo Assay—Compounds found to be active in the ex vivo assay (above) were further tested for their activity in suppressing IgE responses in vivo. Mice receiving low-dose radiation prior to immunization with a carrier exhibited an enhanced IgE response to sensitization with antigen 7 days later. Administration of the test compounds immediately prior to and after antigen sensitization, measured the ability of that drug to suppress the IgE response. The levels of IgE, IgG1 and IgG2a in serum were compared.

Female BALB/cByj mice were irradiated with 250 rads 7 hours after initiation of the daily light cycle. Two hours later, the mice were immunized i.p. with 2 μg of KLH in 4 mg alum. Two to seven consecutive days of drug injections were initiated 6 days later on either a once or twice daily basis. Typically, i.p. injections and oral gavages were administered as suspensions (150 μl/injection) in saline with 10% ethanol and 0.25% methylcellulose. Each treatment group was composed of 5-6 mice. On the second day of drug administration, 2 μg of DNP-KLH was administered i.p. in 4 mg alum, immediately following the morning injection of drug. Mice were bled 7-21 days following DNP-KLH challenge.

Antigen-specific IgE, IgG1 and IgG2a antibodies were measured by ELISA. Periorbital bleeds were centrifuged at 14,000 rpm for 10 min, the supernatants were diluted 5-fold in saline, and centrifuged again. Antibody concentrations of each bleed were determined by ELISA of four dilutions (in triplicate) and compared to a standard curve: anti-DNP IgE (1:100 to 1:800.), anti-DNP IgG2a (1:100 to 1:800), and anti-DNP IgG1 (1:1600 to 1:12800).

In Vitro Measures of Drug Action

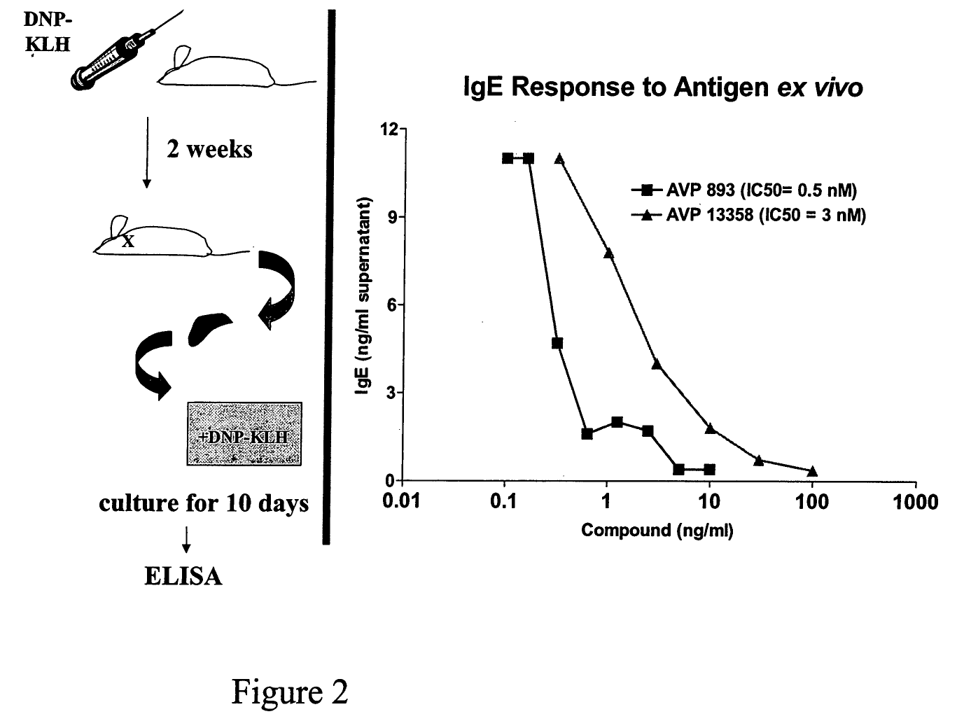

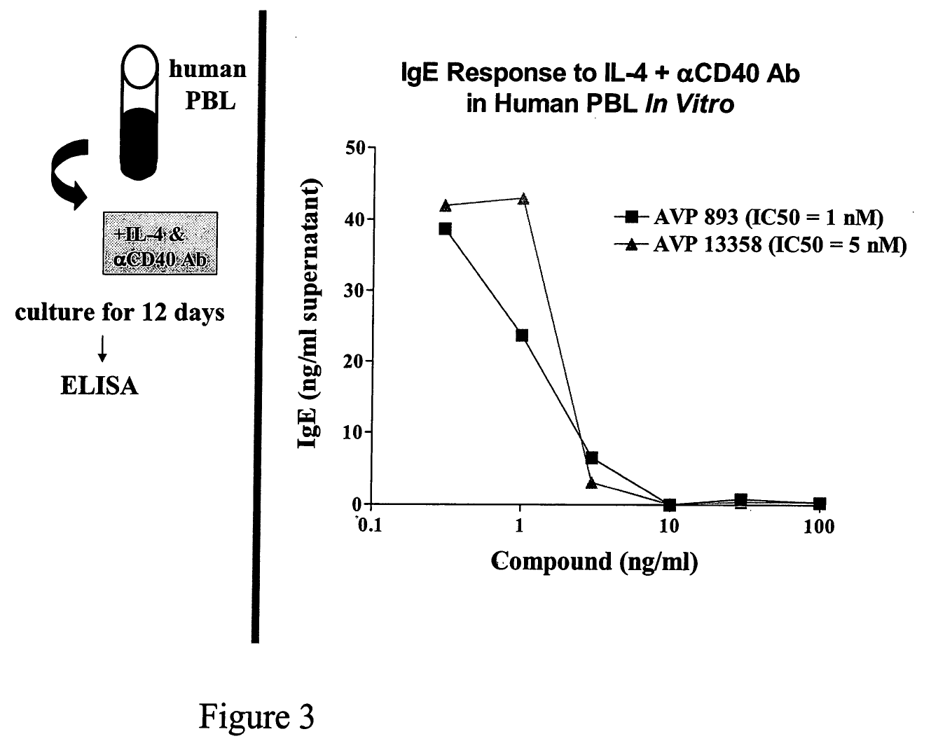

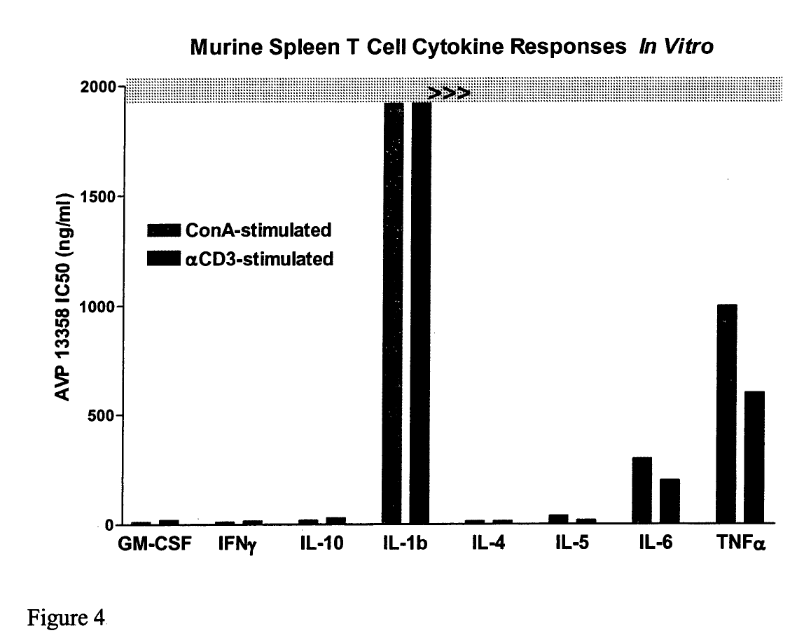

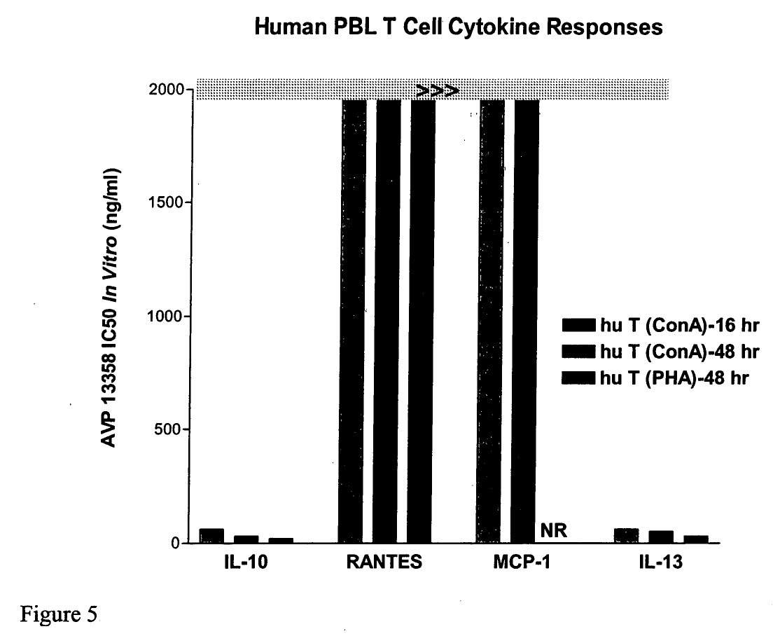

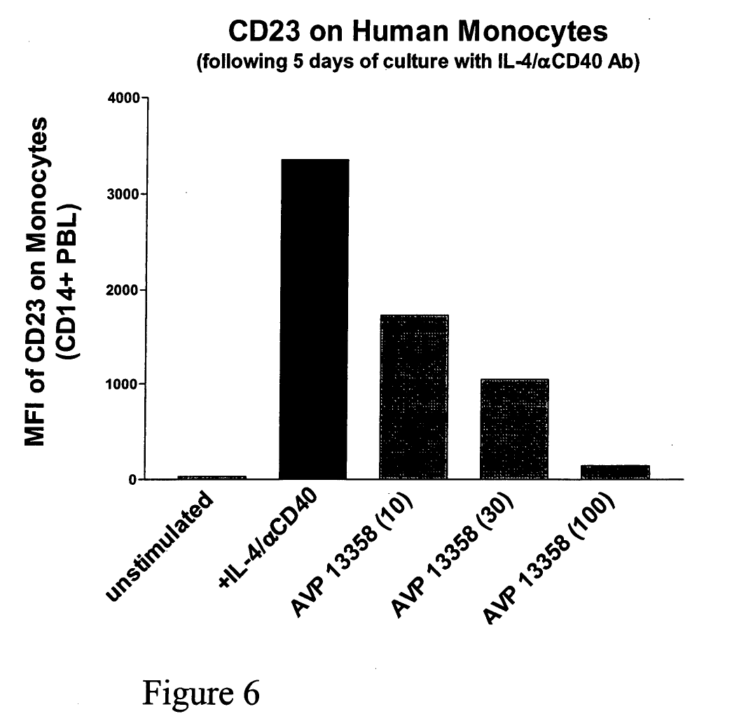

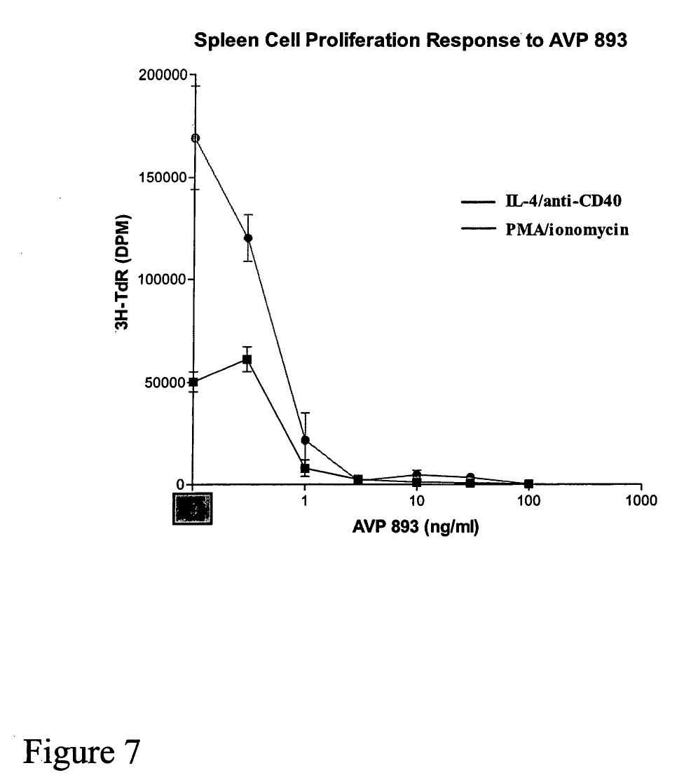

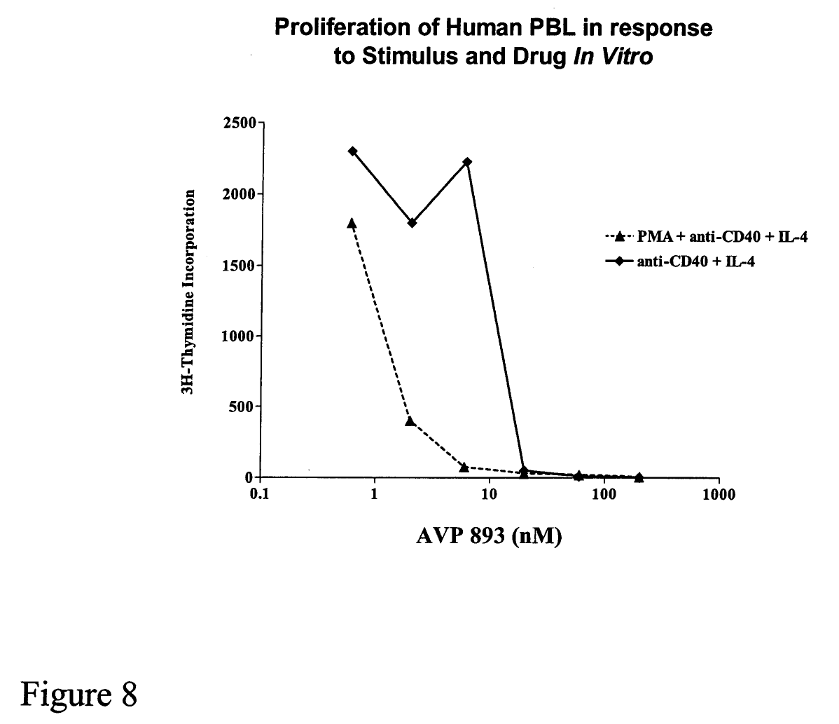

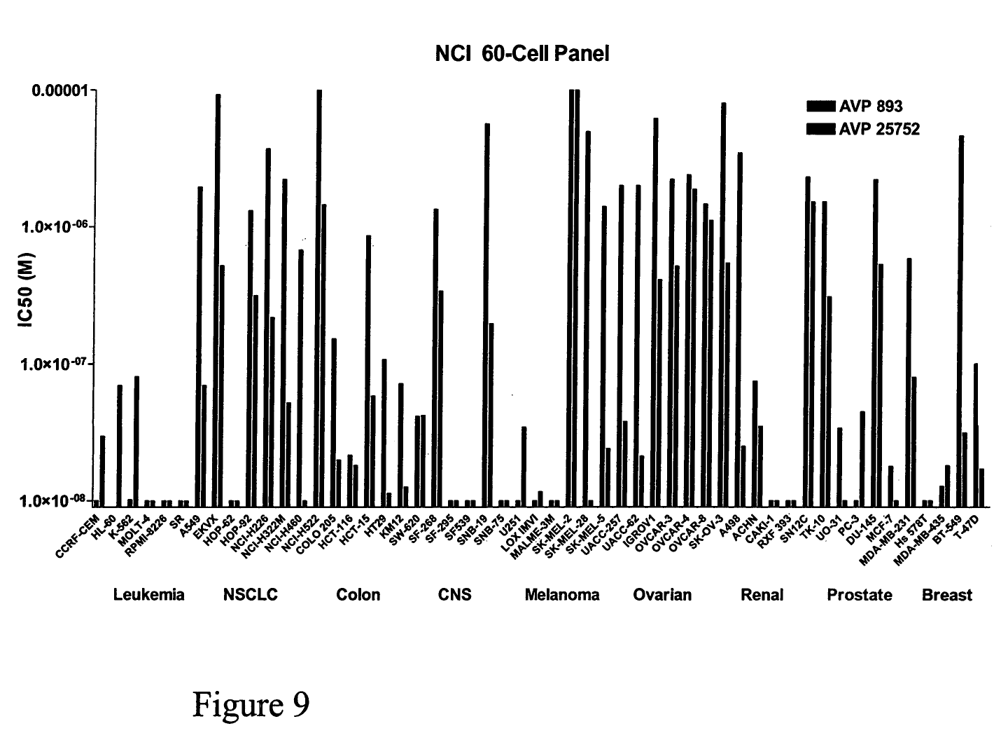

These series of compounds were initially identified on the basis of their IgE-blocking activity in an ex vivo IgE response protocol (FIG. 2), and the biological activity of all analogs are characterized on the basis of their activity in this assay. Activity in the ex vivo assay is corroborated in the in vitro assay of B cell response to 1L-4/anti-CD40 Ab-stimulated IgE in human PBL (FIG. 3) using standard procedures, and mouse splenic B cells (not shown). Drug action on T cells was shown by testing T cell cytokine responses to various stimuli in vitro. The response of a cadre of cytokines and chemokines to several alternative stimuli was tested in T cells from both mouse spleen and human PBL. The data for cytokines that were enhanced at least 10-fold by stimulus are shown in FIGS. 4 and 5. T cells were isolated from murine spleen and cultured for 16 hours in the presence of stimulus+/−AVP 13358. Supernatants were quantified for cytokines using Luminex beads. All cytokines achieved levels of at least 200 pg/ml and 10-fold higher than background (FIG. 4). T cells were isolated from donor PBL and cultured for 16-36 hours in the presence of Phytohemaglutin (PHA, 5 μg/ml) and ConA (5 μg/ml)+/−AVP 13358. Supernatants were quantified for cytokines using Luminex beads (FIG. 5). All cytokines achieved levels of at least 200 pg/ml and these levels were at least 10-fold higher than background. AVP 13358 potently suppressed the levels of most cytokines, including those important for the development of allergy, i.e., IL-4, IL-5, and IL-13. A third group of activities discovered for these compounds is the suppression of membrane receptor expression. Using a similar approach for stimulating the expression of CD23 (the B cell IgE receptor; FIG. 6) and the IL-4 receptor (not shown) as noted above, AVP 13358 potently blocked the induction of these receptors on murine B cells and human monocytes in vitro. The fourth activity discovered for these compounds was the inhibition of cellular proliferation. This effect was noted first in the proliferation of primary cells in response to a variety of stimuli, including IL-4/anti-CD40 Ab, PMA/ionomycin, LPS, ConA, or epidermal growth factor (EGF). Drug effects on the proliferation of mouse spleen cells and human PBL are shown in FIGS. 7 and 8, respectively. Compounds of these series also were shown to have anti-proliferative effects on tumor cell growth in vitro (FIG. 9). AVP compounds were submitted to the NCI for testing in their 60-cell screening panel. The data shown in FIG. 9 represents measures of total protein from 2-day cultures of tumor cell lines. Total protein was assessed by the SRB assay, as adopted for many of the proliferation experiments outline below.

Sulforhodamine B (SRB) Assay Protocol (Adapted from NCI Protocol)

For a typical screening experiment, cells are inoculated into 96 well microtiter plates in 100 μl at plating densities ranging from 5,000 to 40,000 cells/well depending on the doubling time of individual cell lines. After cell inoculation, the microtiter plates are incubated at 37° C., 5% or 10% CO2—depending on the cell line and media—95% air and 100% relative humidity for 24 h prior to addition of experimental drugs. After 24 h, two plates of each cell line are fixed in situ with TCA, to represent a measurement of the cell population for each cell line at the time of drug addition. Following drug addition, the plates are incubated for an additional 48 h at 37° C., 5%/10% CO2, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air-dried. Sulforhodamine B (SRB) solution (10011) at 0.4% (w/v) in 1% acetic acid is added to each well, and plates are incubated for 10 minutes at room temperature. After staining, unbound dye is removed by washing five times with 1% acetic acid and the plates are air-dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, control growth, and test growth in the presence of drug at the five concentration levels], the percentage growth is calculated at each of the drug concentrations levels.

Testing performed at the National Cancer Institute (NCI) revealed the compounds to be novel both in structure and the profile of cells against which the compounds were active.

Corroboration of In Vitro Action by In Vivo Activity.

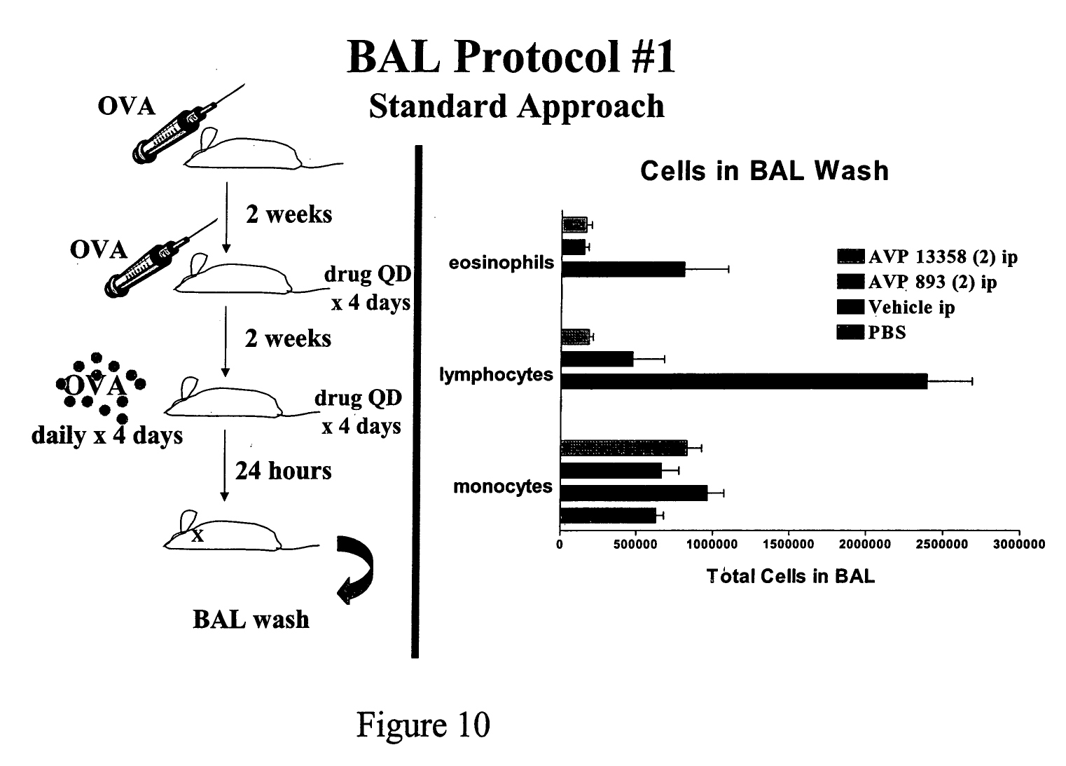

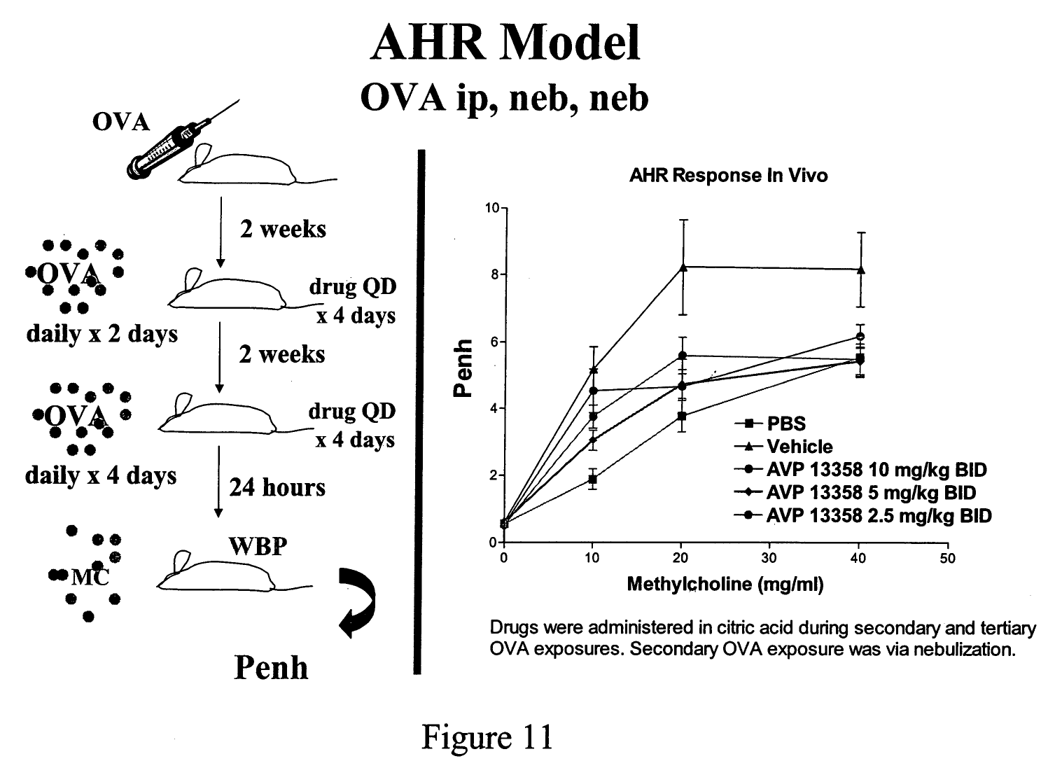

Several of the compounds have been tested in in vivo models of human disease that also reflect the results observed in vitro. Two models of allergic asthma were tested in mice, the broncho-alveolar lavage (BAL) and airway hyper-reactivity (AHR) models. Both models are initiated by a similar protocol to generate an “allergic” response to chicken ovalbumin (OVA). The BAL model measures cellular and cytokine infiltration into the lungs in response to nebulized OVA. Drug administration suppresses the eosinophil and lymphocyte infiltration in the standard protocol (FIG. 10) as well as other similar models. Where increases of cytokines and IgE were noted, drug also suppressed these responses (not shown). Airway hyper-reactivity response to methacholine challenge also was inhibited by drug (FIG. 11). Lastly, B cell expression of CD23 in mice was suppressed by chronic (3 or more consecutive days) treatment with drug in vivo (not shown).

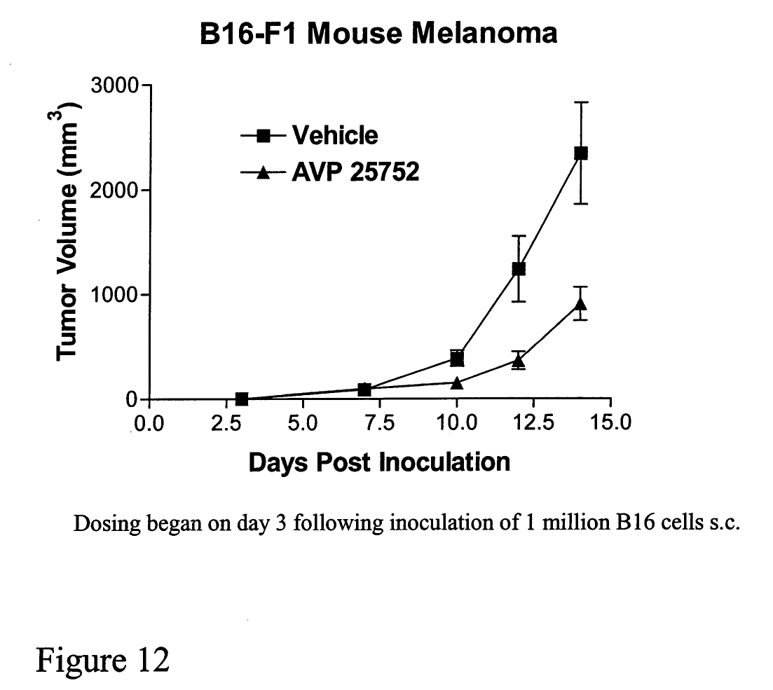

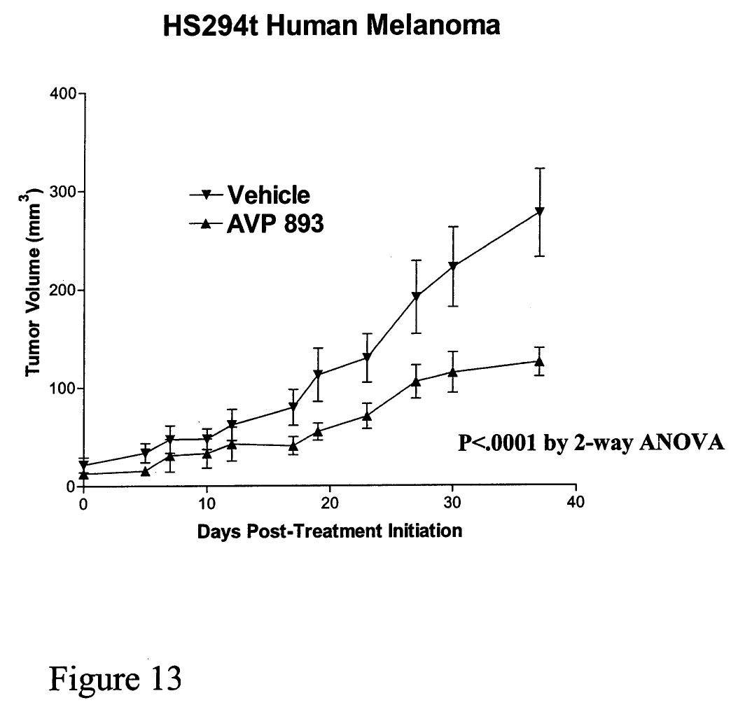

Compounds have been tested for activity in a number of in vivo tumor models. Subcutaneous inoculation of B16 melanoma tumor cells into syngeneic (C57BL/6) mice results in the rapid tumor growth. Drug (AVP 25752) treatment of mice that had been inoculated with tumor cells experienced a significant decrease in the rate of tumor growth compared to vehicle-treated mice (FIG. 12). Similar but more dramatic results were obtained when human melanoma tumor cells were inoculated into Nu/Nu mice in a xenographic model (FIG. 13). Twenty five Nu/Nu mice were inoculated s.c. with 8 million Hst294t tumor cells. Twelve days later mice were separated into two groups and treated with AVP 893 (10-40 mg/kg/day) or vehicle i.p. daily.

Thus, AVP drug effects on the variety of responses observed in vitro are also noted in vivo. This not only provides a level of confidence that the in vitro findings can be carried over to the intact animal, but also indicates that these agents may have utility in treating human diseases wherein these effects would be beneficial.

Screening for Biological Activity

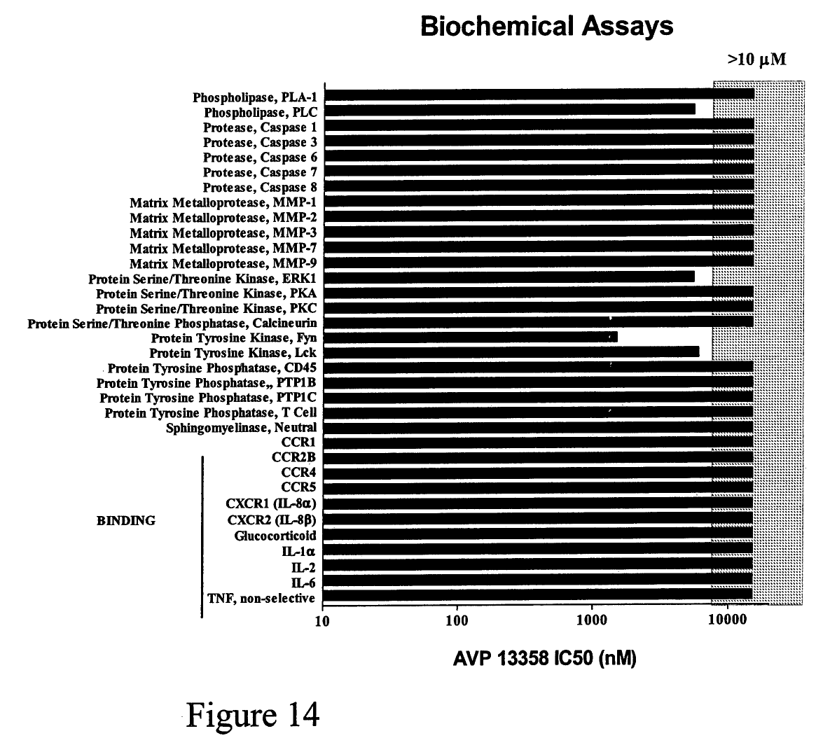

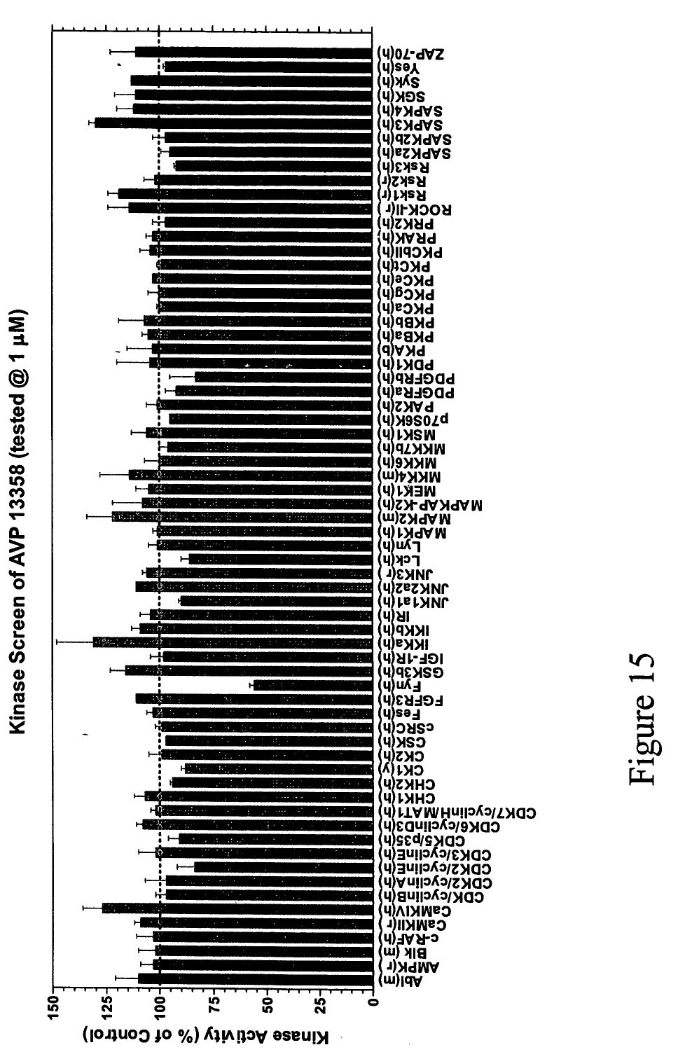

In an effort to understand how these compounds might be acting at the cellular and molecular level, several screens of drug activity were initiated. The first 2 screens were designed to test the activity of drug on certain binding events and the activity of a variety of enzymes in vitro (FIGS. 14 and 15). The results of in vitro biochemical assays (as indicated on the Y-axis) are shown in FIG. 14. The results of a panel of kinase assays, performed by Upstate, Inc. are shown in FIG. 15. AVP 13358 (1 μM) was tested for activity in 60 kinase assays as a part of a screening protocol performed by Upstate Inc. However, no drug activity was observed at concentrations of less than 1 μg/ml, far above it's IC50 for the pharmacological activities described above.

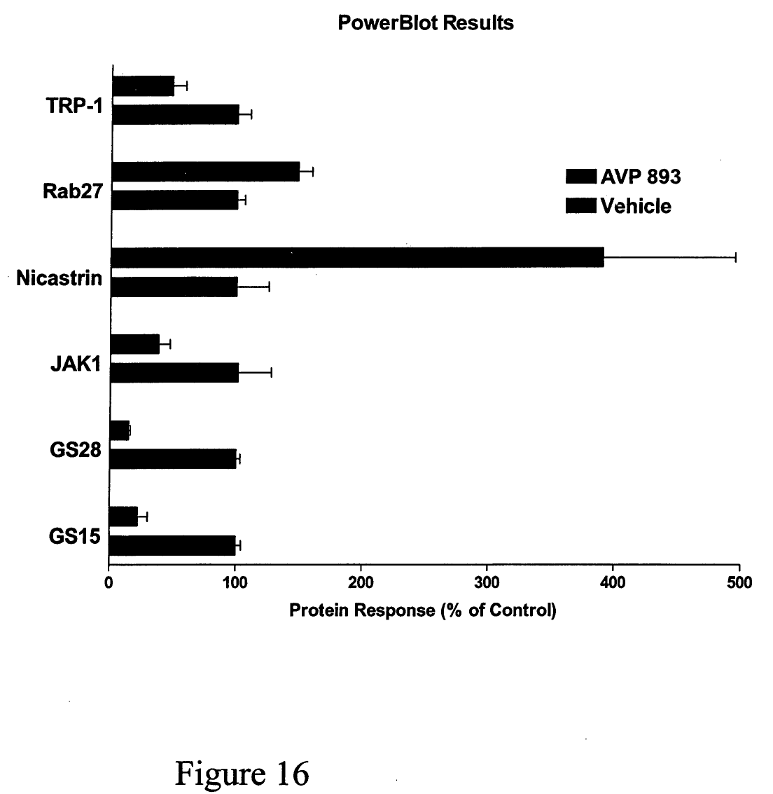

A second series of experiments tested the activity of AVP 893 on the expression of over 950 proteins by Western blotting in vitro (in triplicate); methods detailed below. B16 tumor cells were chosen for this screen and a 16 hour duration of AVP 893 treatment was selected to optimize the number of proteins that might be modified by drug. Only 6 proteins were found to be consistently and significantly modified in lysates derived from drug-treated cells (FIG. 16). B16-F10 cells were cultured for 16 hr in the presence or absence of 100 ng/ml AVP 893. Samples were harvested and lysates prepared according to instructions supplied by Becton-Dickinson. Samples were placed on dry ice and submitted to the same for expression analysis of 950 proteins. Of these, only GS28 and nicastrin were found to be consistent changes in the B16 and other cell lines. Although both proteins have entirely different functions in the cell, and have not been linked (apparently) in the scientific literature, there is a rational explanation for the changes noted in each protein, as described below.

Western Blotting and Sample Preparation

The culture medium was removed by vacuuming (for attached cells) or by low speed centrifugation (for suspension cells) for 5-7 minutes at room temperature. The cells were wasedh twith PBS, spun at 1200 rpm and the cell pellets were kept on ice. 300 μl/2.0×10⁷ cells of ice cold lysis buffer was added with freshly added protease inhibitors. Cell pellets were gently resuspended and incubated on ice for at least 30 min, vortexed a few times during incubation. Cell lysate was spun at 14,000 rpm for 2-5 min at 4° C. The supernatant was transferred to a new microfuge tube and the pellet was discarded. An aliquot of sample was mixed with an equal volume of 2× sample buffer (InVitrogen), and stored at −80° C. Protein concentration was determined by using “BCA protein assay reagent kit” from Pierce.

Electrophoresis and Transfer

Protein samples (in sample buffer) were boiled for 1-3 minutes and put on ice. Same amount of protein were loaded on the NuPage gel (InVitrogen). After the electrophoresis was complete, proteins were transferred from the gel to a PVDF membrane using the electro-blotting apparatus from InVitrogen; the voltage was set to 25 for 2-3 hr. Block non-specific binding by incubating membrane with 5% milk (in PBS, 0.1% tween 20) for at least 30 min at room temperature or overnight at 4° C. The blocked membrane was incubated with primary antibody (See TABLE 2) diluted in 5% milk for 1 hour at room temperature. Optimal antibody dilution depends on the company, the amount of protein. Dilutions of 1:1000 were generally used for the primary antibodies from Santa Cruz. The membrane was washed with PBS, 0.1% tween 3-4 times 5 mins. The membrane was incubated for 30-60 minutes at room temperature with horseradish peroxidase (HRP) conjugated secondary antibody diluted in 5% milk. We usually used 1:4000 dilution for the secondary antibody from Santa Cruz. The membrane was washed 3-4 times with PBS, 0.1% tween, each time 15 minutes. The detection solutions A and B were mixed in a ratio 40:1 and Pipetted onto the membrane, and incubated for 5 min at RT. A sheet of Hyper film ECL was placed on the top of the membrane in the dark and exposed for 1 min, or adjust accordingly.

TABLE 2

| Name | Cat # | | Species | company source |
|---|---|---|---|---|
| primary antibodies | | | | |
| ARF (H-50) | sc-9063 | rabbit polyclonal | m, r, h | Santa Cruz |
| Y1-adaptin (M-300) | sc-10763 | rabbit polyclonal | m, r, h | Santa Cruz |
| Bet1 | 612038 | mouse monoclonal | m, r, h, d, chick | BD bioscience |
| Copβ (T-14) | sc-13335 | goat polyclonal | m, r, h | Santa Cruz |
| Calnexin (C-20) | sc-6465 | goat polyclonal | m, r, h | Santa Cruz |
| EEA1 (N-19) | sc-6415 | goat polyclonal | m, h | Santa Cruz |
| E-Cadherin (H-108) | sc-7870 | rabbit polyclonal | m, r, h | Santa Cruz |
| Copε (E-20) | sc-12104 | goat polyclonal | m, r, h | Santa Cruz |
| ErbB-4 (C-18) | sc-283 | rabbit polyclonal | m, r, h | Santa Cruz |
| GS27 (G-20) | sc-14157 | goat polyclonal | m, r, h | Santa Cruz |
| GS15 | 610960 | mouse monoclonal | d, Hu, Ms, r, Bov, Frog | BD Bioscience |
| GS28 | 611184 | mouse monoclonal | m, r, h | BD Bioscience |
| GS28(N-16) | sc-15270 | rabbit polyclonal | m, r, h | Santa Cruz |
| HCAM (H300) | sc-7946 | rabbit polyclonal | m, r, h | Santa Cruz |
| HSV-1VP16(vA-19) | sc-17547 | goat polyclonal | HSV-1 protein | Santa Cruz |
| HSV-2glycoproteinD(vl-20) | sc-17538 | goat polyclonal | HSV-2 protein | Santa Cruz |
| HCAM (DF1485) | sc-7297 | mouse monoclonal | h | Santa Cruz |
| Histone H1(FL-219) | sc-10806 | rabbit polyclonal | broad | Santa Cruz |
| NSF (C-19) | sc-15917 | goat polyclonal | m, r, h | Santa Cruz |
| NSF (N-18) | sc-15915 | goat polyclonal | m, r, h | Santa Cruz |
| Notch1(H-131) | sc-9170 | rabbit polyclonal | m, r, h | Santa Cruz |
| Nicastrin (N-19) | sc-14369 | goat polyclonal | m, r, h | Santa Cruz |
| Presenilin 1 (N-19) | sc-1245 | goat polyclonal | m, r, h | Santa Cruz |
| Presenilin 2 (C-20) | sc-1456 | goat polyclonal | m, r, h | Santa Cruz |
| Rab5A (S-19) | sc-309 | rabbit polyclonal | m, r, h | Santa Cruz |
| Rab1A (C-19) | sc-311 | rabbit polyclonal | m, r, h | Santa Cruz |
| Rab1B (G-20) | sc-599 | rabbit polyclonal | m, r, h | Santa Cruz |
| Rab2 (P-19) | sc-307 | rabbit polyclonal | m, r, h | Santa Cruz |
| Rab8 (P-16) | sc-306 | rabbit polyclonal | h | Santa Cruz |
| Rab6 (C-19) | sc-310 | rabbit polyclonal | m, r, h | Santa Cruz |
| SNAP25 (N-19) | sc-7539 | goat polyclonal | m, r, h | Santa Cruz |
| SYP (C-20) | sc-7568 | goat polyclonal | m, r, h | Santa Cruz |
| Syntaxin (FL-288) | sc-13994 | rabbit polyclonal | m, r, h | Santa Cruz |
| Syntaxin-1 (HPC-1) | sc-12736 | mouse monoclonal | m, r, h | Santa Cruz |
| Ykt6p (K-16) | sc-10835 | goat polyclonal | m, r, h | Santa Cruz |
| α-SNAP (N-19) | sc-7770 | goat polyclonal | m, r, h | Santa Cruz |
| SNAP 23 | 111 202 | rabbit polyclonal | m, h | SYSY, Germany |
| SNAP 23A | VAP-SV013 | rabbit polyclonal | hu, ha, ca, bov | stressgen |
| β-Tubulin (D-10) | sc-5274 | mouse monoclonal | m, r, h | Santa Cruz |
| VAMP-1 (FL-118) | sc-13992 | rabbit polyclonal | m, r, h | Santa Cruz |
| VAMP-3 (N-12) | sc-18208 | goat polyclonal | m, r, h | Santa Cruz |
| p115 (N-20) | sc-16272 | goat polyclonal | m, r, h | Santa Cruz |
| secondary antibodies | | | | |
| Rabbit anti-goat IgG-HRP | sc-2768 | | | Santa Cruz |
| Goat anti-rabbit IgG-HRP | sc-2004 | | | Santa Cruz |
| anti-mouse IgG-HRP | sc-2005 | | | Santa Cruz |
| Goat anti-mouse IgG-HRP | sc-2005 | | | Santa Cruz |

Expression of Cell Trafficking Proteins

GS28 is a t-SNARE protein that is involved in the docking and fusion of vesicles in the golgi and the intermediate compartment (IC, located between the ER and golgi). Thus, GS28 is intimately involved in the movement of proteins (via vesicles) both between the ER and golgi and within the golgi cistemae. Nicastrin is a part of the γ-secretase complex that is responsible for intramembrane cleavage of a number of proteins that subsequently translocate into the nucleus and act as transcription factors. Included amongst these proteins are amyloid precursor protein (APP), Notch, erbB4, E-cadherin, and others. Drug treatment of B16 cells results in a block of nicastrin maturation such that the immature, partially glycosylated form of nicastrin accumulates at the expense of the fully glycosylated active moiety. Nicastrin normally passes through the ER where it its partially glycosylated and then to the golgi where glycosylation and sialation is completed. Thus nicastrin is essentially acting as a cargo protein whose changes are reflective of how it moves through the cell. By suppressing the maturation of nicastrin, AVP 893 treatment appears to prevent the ER-to-golgi trafficking of nicastrin, perhaps through its effect on GS28.

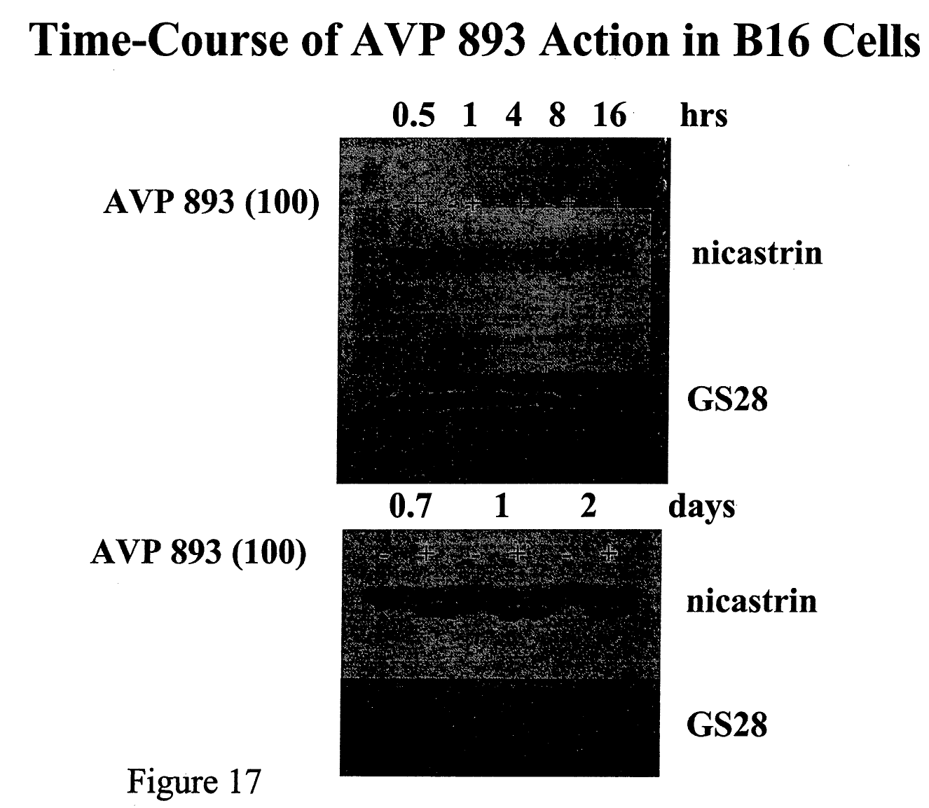

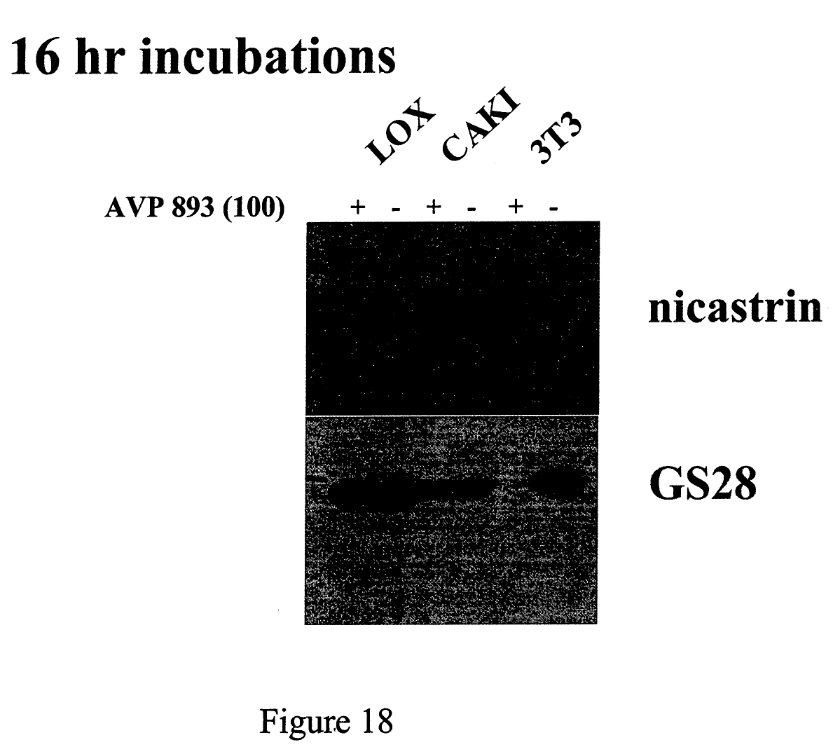

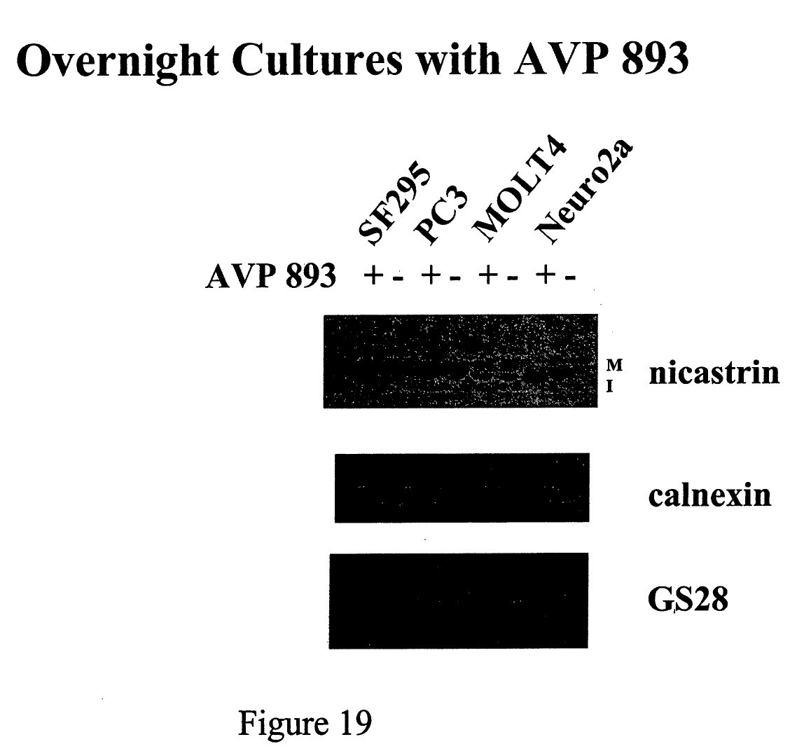

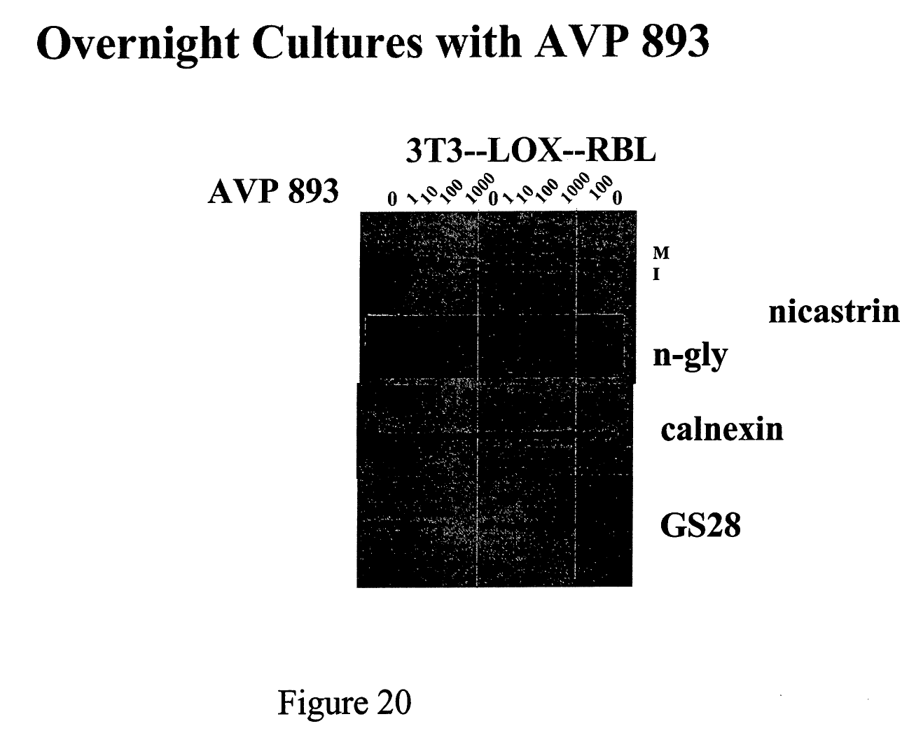

To further examine the putative protein trafficking effects of AVP 893, other proteins in this pathway were tested in vitro in B16 and other cell lines. The effect of AVP 893 on cellular proteins was corroborated in B16 cells and extended to include a time-course (FIG. 17). B16-F10 tumor cells were seeded in T75 flasks at 20% confluence and cultured overnight. AVP 893 (100 ng/ml) was added to several flasks and one flask of cells was harvested at several time points following addition of compound. Lysates were prepared, separated by electrophoresis, and probed with antibody as described above in the general Western blotting protocol. Drug effects on GS28 and nicastrin paralleled each other and were progressively stronger with longer drug incubations. Two days of culture with AVP 893 resulted in a complete loss of GS28. Other cell lines were tested for their expression of GS28 and nicastrin and found to respond similarly to drug, although quantitative differences were evident. Tumor cell lines found to respond similarly to AVP 893 include CAK1, SF295, PC3, MOLT4, Neuro2a, and RBL (FIGS. 18, 19, and 20). For the experiment shown in FIG. 18, LOX, CAKI, and 3T3 cell lines were treated as described for FIG. 17. For the results shown in FIG. 19, SF295, PC3, MOLT-4, and Neuro2a cells were treated as described for FIG. 17. For the results shown in FIG. 20, LOX, 3T3, and RBL cell lines were treated with varying concentrations of AVP 893 as described for FIG. 17. The effects on LOX cells were less evident. The normal fibroblast cell line, 3T3, showed a more profound response to drug as levels of GS28 and mature nicastrin were virtually eliminated by AVP 893 exposure. Levels of calnexin, a resident ER protein used as a control, were unchanged in drug-treated cells. An AVP 893 concentration/response evaluation for 3T3 cells suggests that the IC50 for GS28 and mature nicastrin expression is between 10 and 100 ng/ml (FIG. 20), which is consistent with the IC50 for AVP 893 inhibition of 3T3 cell proliferation.

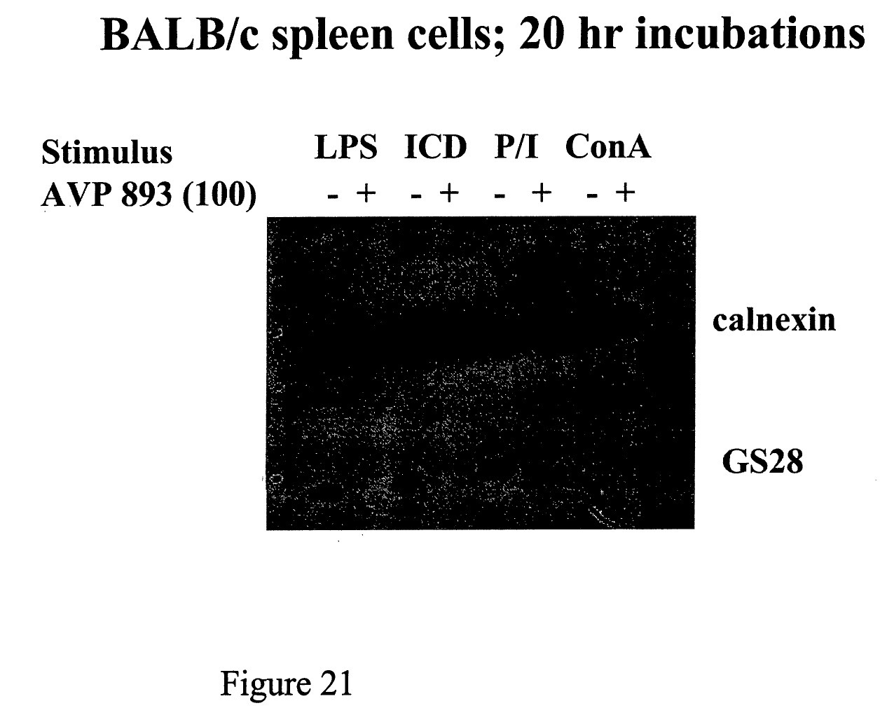

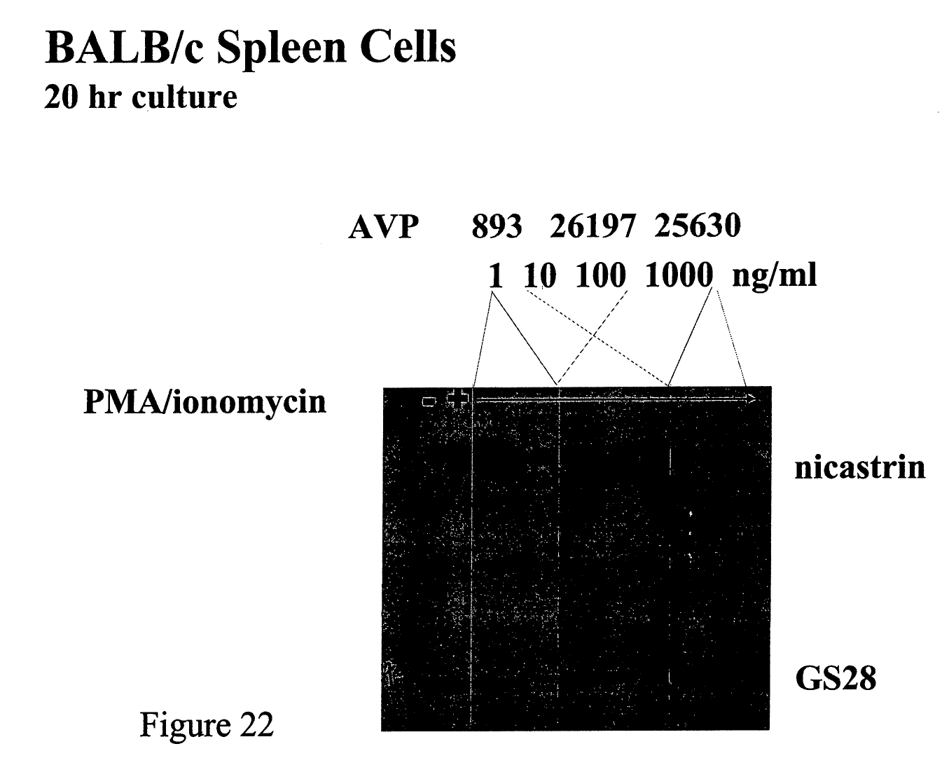

AVP 893 also suppressed GS28 expression in mouse spleen cells that were stimulated with various stimuli (FIG. 21). BALB/c spleen cells were cultured for 20 hours in the presence of stimulus+/−AVP 893 (100 ng/ml) and harvested and prepared as described in FIG. 17. Stimulus conditions include: LPS (10 μg/ml), IL-4 (10 ng/ml) plus anti-CD40 Ab (100 ng/ml), PMA (10 ng/ml) plus ionomycin (100 nM), or Con A (5 μg/ml). As with the 3T3 fibroblasts, spleen cell expression of GS28 was abrogated by drug while calnexin expression was minimally affected. FIG. 22 compares the effects of 3 compounds that possess different potencies for inhibition of IL-4/anti-CD40 Ab-stimulated IgE production or proliferation by mouse spleen cells. This experiment was carried out as described for FIG. 21, except that different AVP compounds with high (AVP 893, 5 nM), medium (AVP 26297, 50 nM), and low (AVP 25630, 500 nM) anti-proliferative potency were tested and compared. AVP 893 was tested at 1, 10, and 100 ng/ml; AVP 26297 was tested at 1, 10, 100, and 1000 ng/ml; AVP 25630 was tested at 10, 100, and 1000 ng/ml. For each compound, the effect on both GS28 and mature nicastrin paralleled their effect on proliferation in vitro suggesting that these effects at the cellular and proteins level are linked.

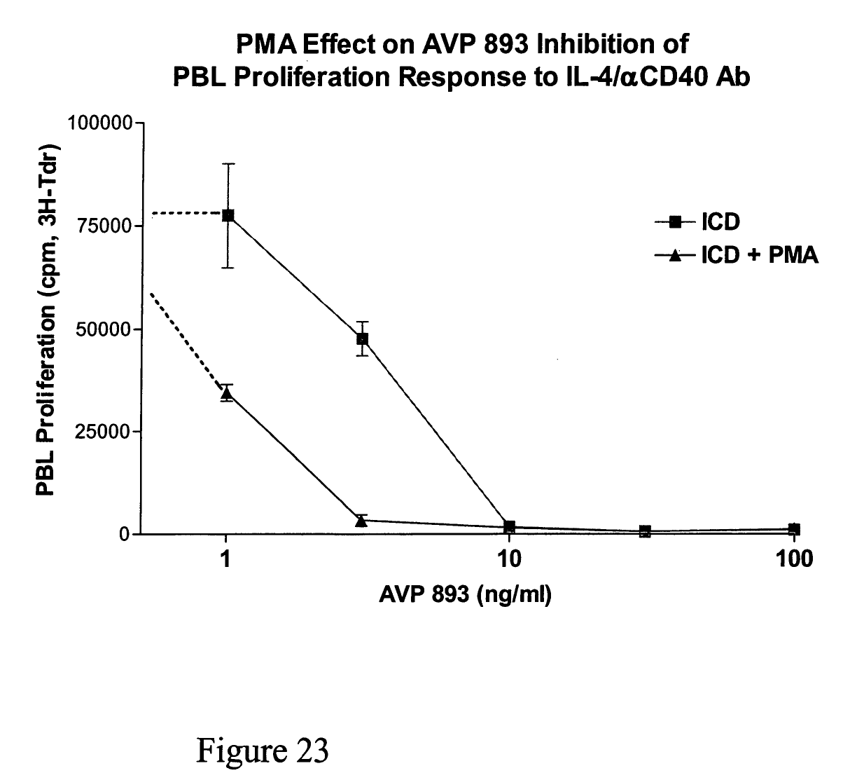

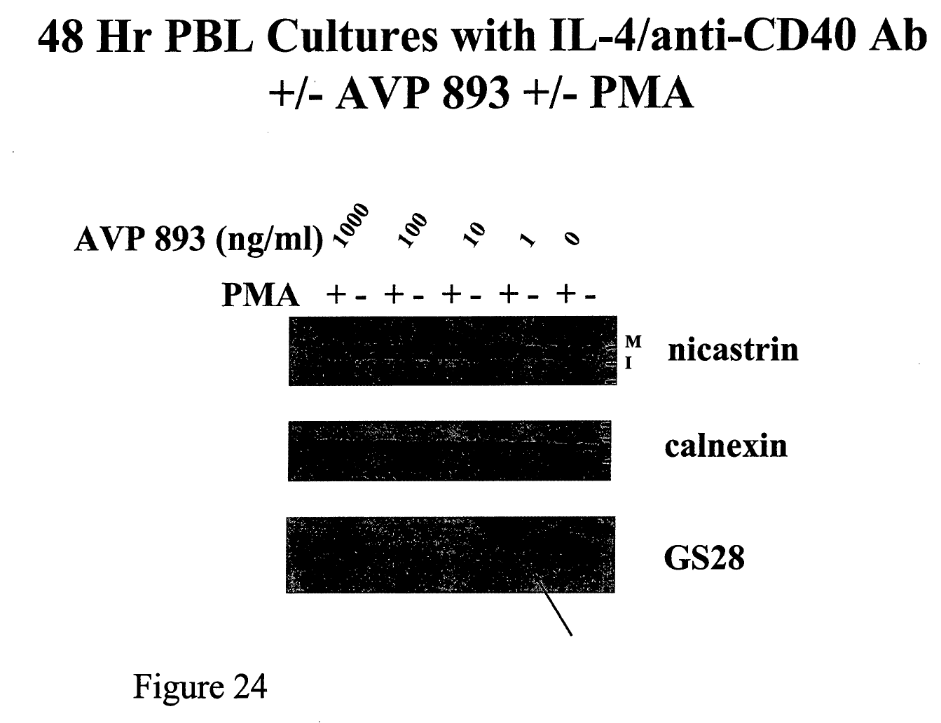

A similar experiment performed on mouse spleen cells was repeated in human PBL except that some samples were also treated with the protein kinase C activator, PMA. The addition of PMA to EL-4/anti-CD40 Ab in in vitro cultures does not affect the proliferation of human PBL or their IgE response but does enhance the potency of AVP 893 for inhibiting both measures (FIG. 23). PBL were prepared and cultured in the presence of stimulus+/−AVP 893 for 4 days before pulsing with 3H-Thymidine and harvesting. Stimulus conditions were either IL-4/anti-CD40 Ab or the combination of PMA and IL-4/anti-CD40 Ab. For these cultures, the following concentrations of human-specific reagents were used: PMA (100 ng/ml), IL-4 (100 ng/ml), and anti-CD40 Ab (300 ng/ml). Similarly, the addition of PMA to PBL does not increase the level of GS28 but enhances the potency of AVP 893 for inhibiting GS28 expression (FIG. 24). PBL cultures were carried out as described for FIG. 23 except that the cells were harvested after 48 hours and lysates prepared for Western blotting (as in FIG. 17). These results provide additional evidence for the existence of a link between the cellular effects of AVP 893 and GS28 expression in primary (non-transformed) cells.

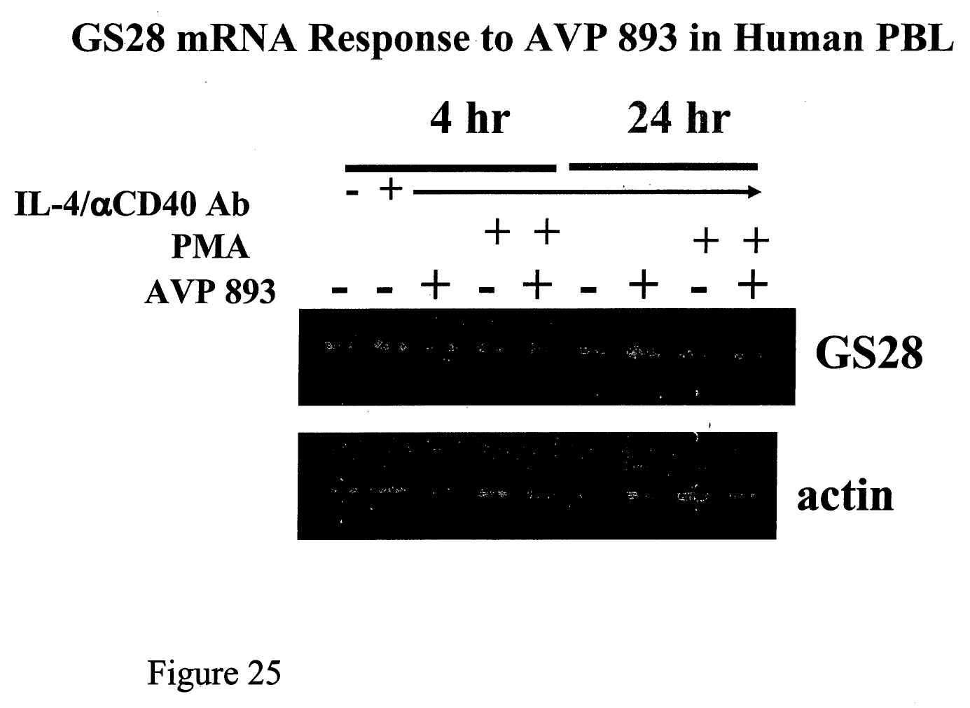

The specific mode by which AVP 893 diminishes expression of GS28 protein is not yet known but does not appear to involve transcription, as AVP 893 did not affect the level of GS28 mRNA when tested 3 to 16 hours following addition of drug (FIG. 25). Human buffy coats were purchased from the San Diego Blood Bank. Buffy Coat was purified of red blood cells using Histopaque-1077 following Sigma Diagnostic protocol. Lymphocytes (20 million) were then cultured in 75 cm2 flasks in cDMEM (+/−stimulus & AVP 893) for either 4 or 24 hrs. Cells were harvested and reconstituted in a Guanidine/Phenol solution essentially as described by Maniatis. The aqueous layer was removed and washed with Guanidine solution and finally 70% EtOH. RNA purity was checked by spectrophotometer. RT-PCR (36 cycles) was performed following the RT-PCR One-Step protocol (Qiagen). Similar results were obtained when testing mRNA samples obtained from other cell sources (not shown).

| | |
|---|---|
| Primers | |
| GS28 | 5′-GATCTCAGGAAACAGGCTCG-3′, |
| | 5′-CCTGTAAGCCTTGCCAAAAG-3′ |
| ACTIN | 5′-GTGGGCCGCTCTAGGCACCA-3′ |
| | 5′-TGGCCTTAGGGTGCAGGGGG-3′ |

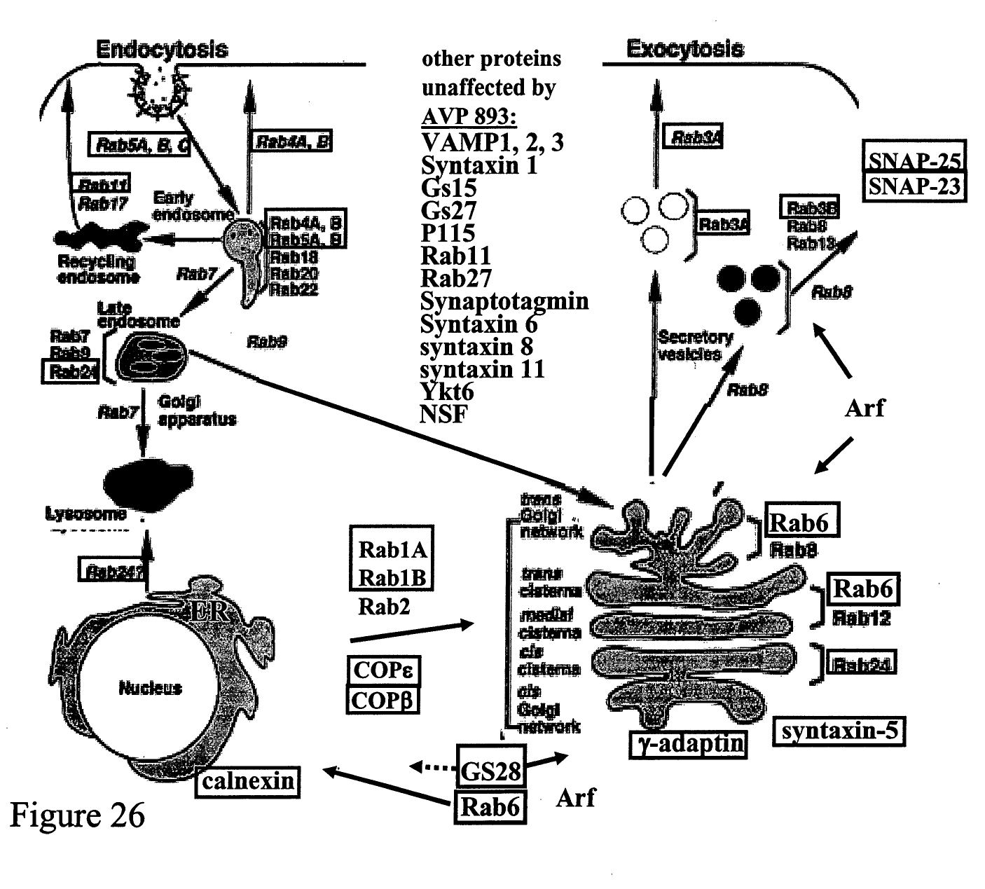

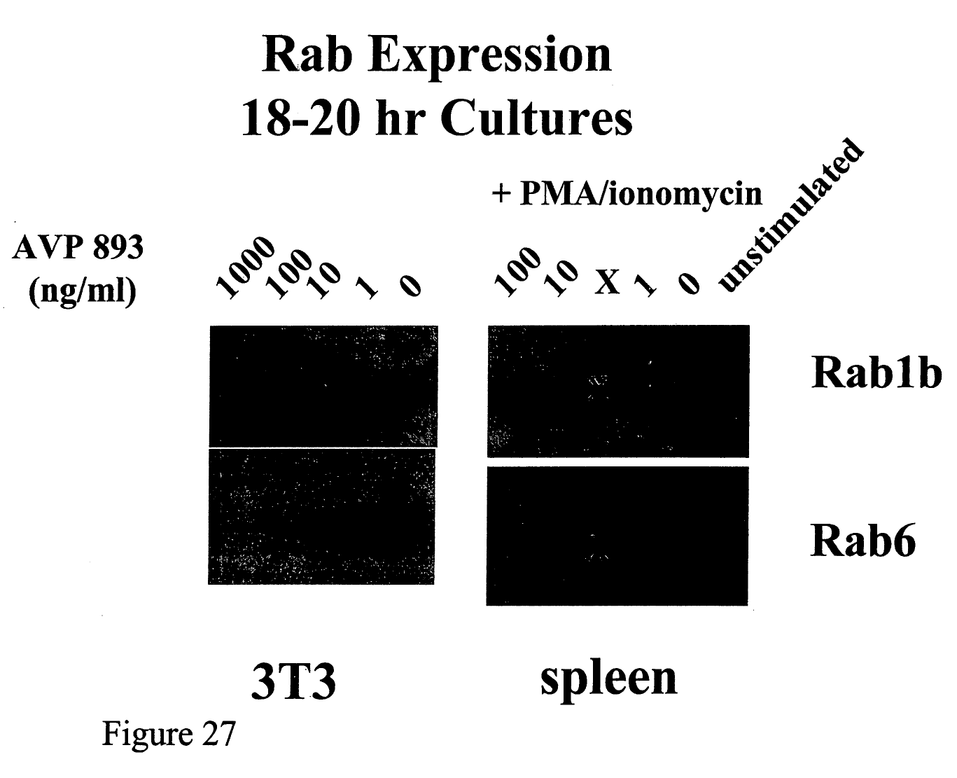

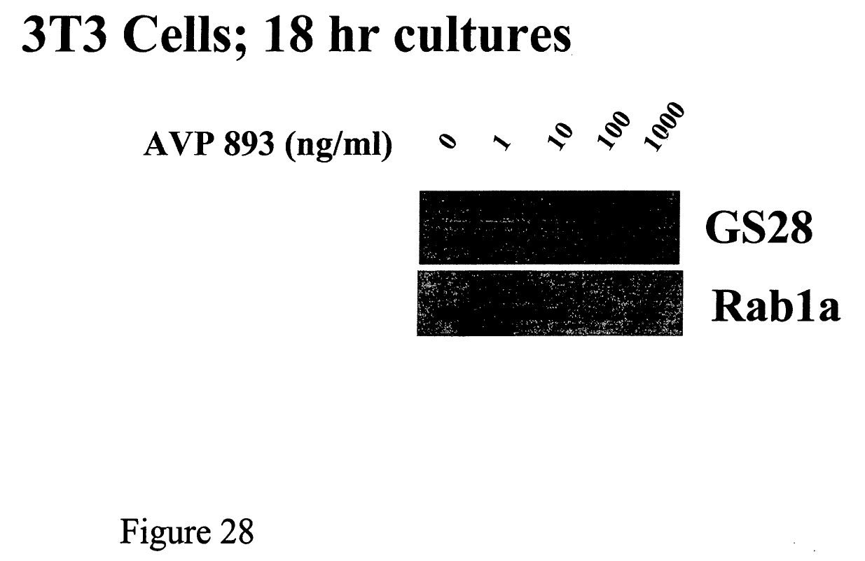

GS28 is but one member of a complicated pathway of interacting proteins that are responsible for the movement of vesicles through the cell. In addition to the SNARE proteins that are involved in vesicular docking and fusion, a group of small Ras-like GTPases known as Rabs are responsible for activating many of these proteins to permit their interaction. Rab proteins known to play a prominent role in the ER-golgi protein trafficking include Rab1a, Rab1b and Rab6 (FIG. 26). Both Rab1 proteins help COPII protein-coated vesicles to travel from the ER to the golgi, while Rab6 is involved in the retrograde movement of vesicles back to the ER. Consistent with the effect on GS28, AVP 893 also suppressed Rab6 expression in 3T3 and PMA/ionomycin-stimulated spleen cells in vitro (FIG. 27). 3T3 fibroblasts and BALB/c spleen cells were cultured overnight with AVP 893 and harvested as noted for FIG. 17. Spleen cells were cultured in the presence and absence of PMA/ionomycin as described for FIG. 21. The response of Rab1 differed depending upon the cell; Rab1b was suppressed in spleen cells by drug but not affected in 3T3 cells while Rab1a showed a mild response to drug in 3T3 cells (FIGS. 27 and 28).

The effect of AVP 893 on the expression of an array of other trafficking proteins was also tested but no other proteins appeared to be modulated quantitatively, including several of the putative interacting partners of GS28 (VAMPI, Gs15, Ykt6) and a variety of tethering proteins and GTPases (FIG. 26). Most of these proteins function outside of the ER-golgi region while the locations of many have not been defined.

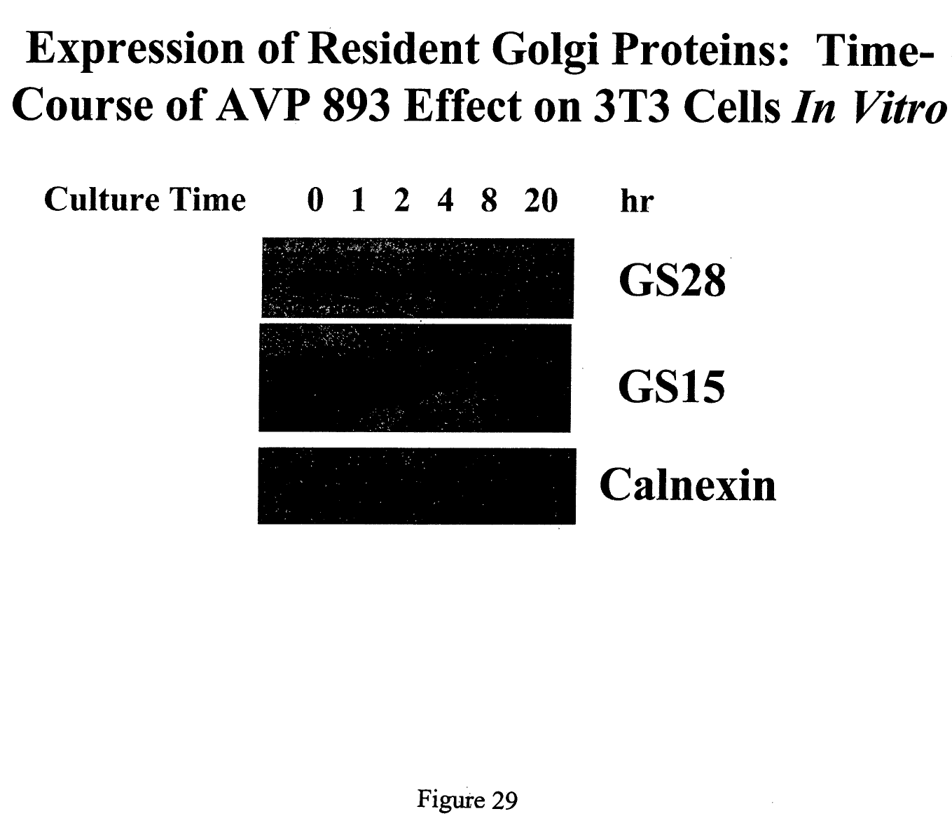

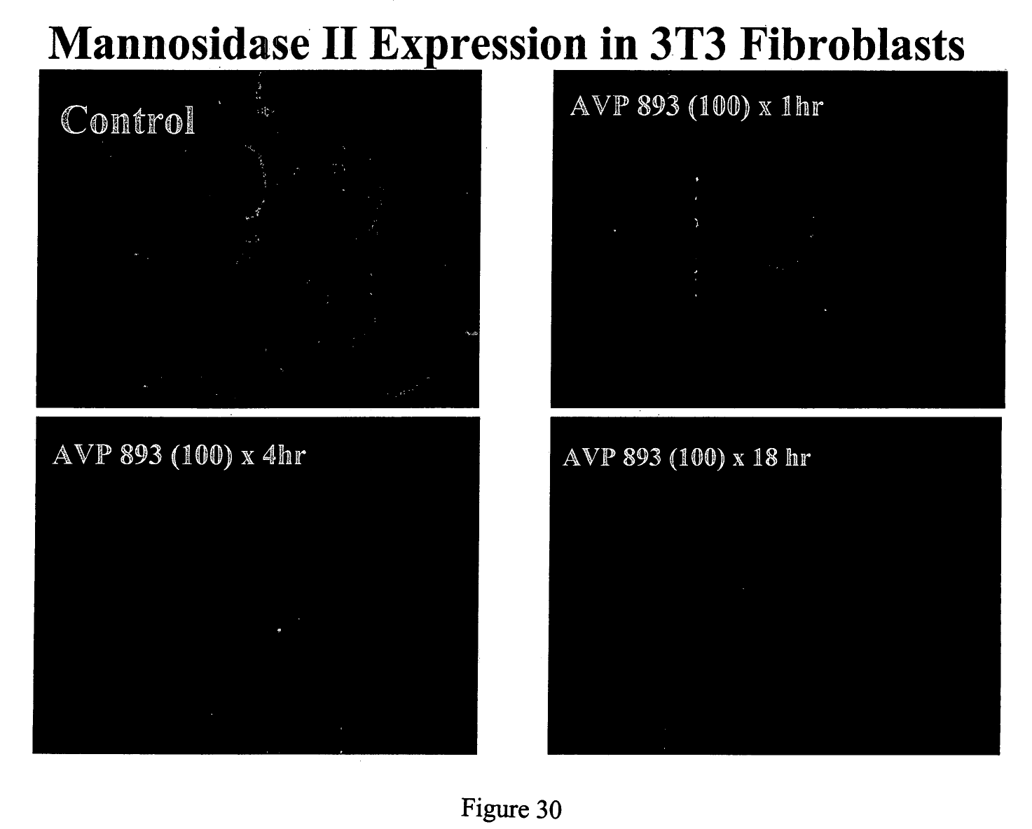

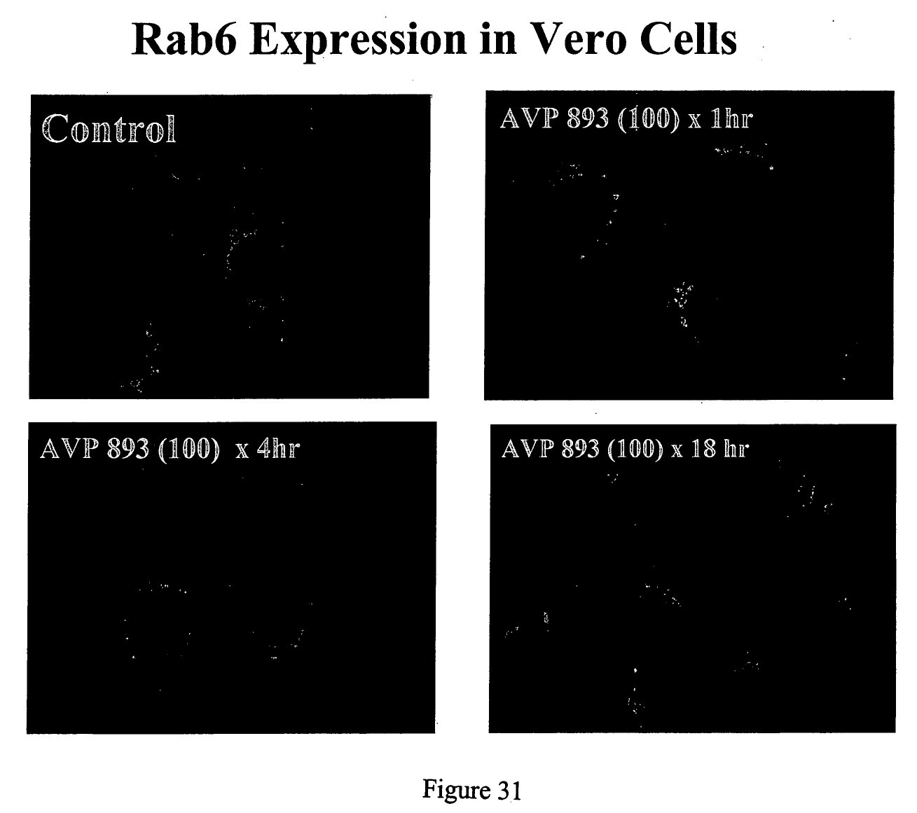

AVP 893 was found to affect the quantitative expression of resident golgi proteins such as GS28 and GS15 in a time-dependent manner, as shown in FIG. 29, as well as Mannosidase II (FIG. 30) and GPP130 (data not shown). GS15 staining in 3T3 cells was greatly diminished by AVP 893 beginning around 2 to 4 hrs of exposure, whereas GS28 levels started dropping off after 8 hrs of exposure, culminating in significantly reduced levels after 20 hrs of drug incubation. GM-130, a golgi-structural protein, did not appear to be affected by AVP 893 (data not shown). Likewise, the non-resident golgi protein Rab6 appeared to be unaffected in some cell types, as illustrated in FIG. 31.

These results demonstrate that AVP 893 acted discriminately on the expression of resident golgi proteins. GS15, GS28, GPP130, and Mannosidase II, sparing the golgi structural protein GM-130 and having little effect on the Rab GTPase Rab6. Furthermore, these affects were most pronounced following overnight (16-20 hr) incubations with AVP 893, although some affects at early time points were seen. These conclusions were drawn from the western blot analysis (FIG. 29), as well as from the immunocytochemical studies (FIGS. 30-31). More particularly, Mannosidase II, a resident golgi enzyme involved in carbohydrate processing, was shown to diminish (FIG. 30) in golgi beginning after 1 hr of AVP 893 application, with little to no discernible amount of the enzyme remaining after 4 hrs, and certainly none after 18 hrs. In contrast, as shown in FIG. 31, the staining of the GTPase Rab6 was not diminished nor significantly altered by the presence of AVP 893, even after 18 hrs.

Accordingly, it can be concluded that AVP 893 discriminately affects golgi resident proteins while leaving non-resident proteins (e.g. Rab6) or structural proteins, such as GM-130 (data not shown), unaffected. In addition, the Mannosidase II data is yet another example of the time course of AVP 893 action on resident golgi proteins, wherein a slow decrease in expression levels culminates in severely diminished levels after 16-20 hrs of drug incubation.

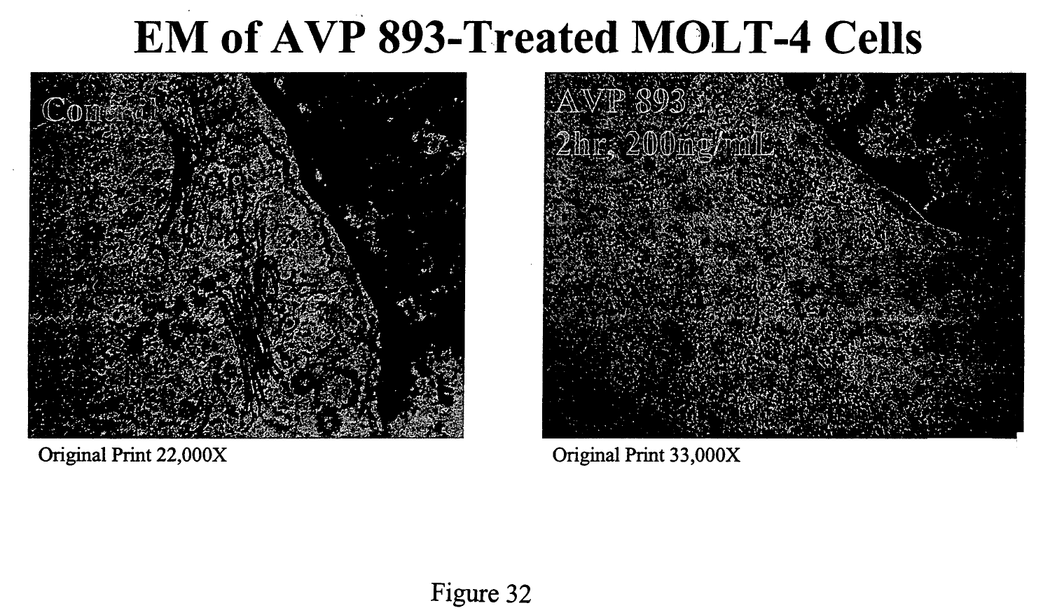

Experiments were conducted to examine the golgi structure and morphology on the ultrastructural level following treatment with AVP 893. Electron microscopic analysis of untreated MOLT4 cells vs. MOLT4 cells treated with AVP 893 (200 ng/mL) for 2 hrs or 18 hrs demonstrated that AVP 893 disrupts golgi structure (FIG. 32). At 2 hrs of AVP 893 treatment, and after 18 hrs treatment (data not shown), no golgi cisternae were found. This finding was repeated with Vero cells, where AVP 893 was applied for 1 hr, 4 hrs, and 18 hrs, with the later two exposures resulting in disruption of cisternal structure (data not shown). We therefore conclude that AVP 893 disturbs the structure of the golgi cistemae within a few hours of treatment.

Intracellular Protein Movement

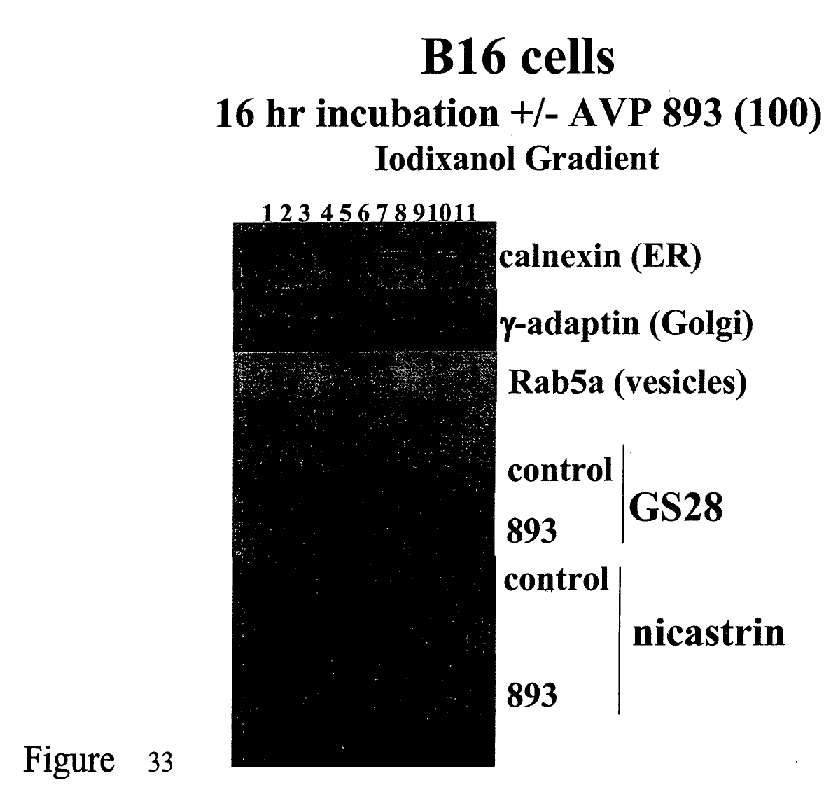

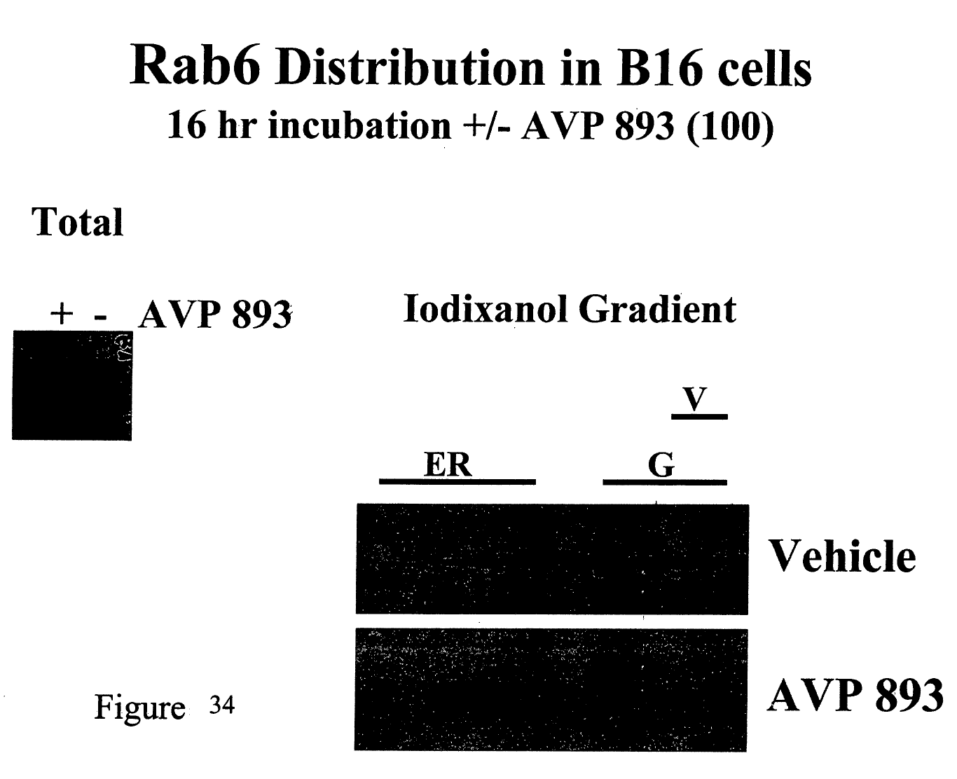

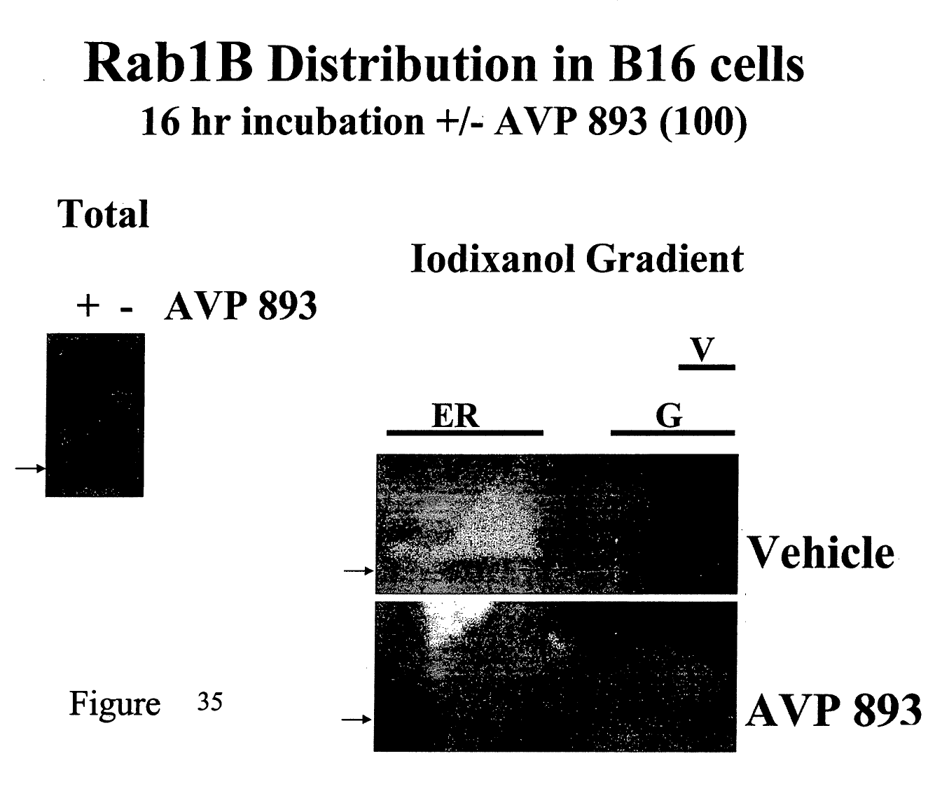

An effect on protein movement through the ER-golgi is suggested by the selective inhibition on trafficking proteins within this region. To test this possibility directly, cells were cultured with and without drug for 16-20 hours, harvested, lysed, and layered on top of a gradient of varying density iodixanol-containing fractions (2.5-30%). The gradients were centrifuged for 2 to 18 hours at 56K x g, collected, and tested for resident proteins via Western blot. Fractions were probed with antibodies specific for calnexin (ER-specific marker), γ-adaptin (golgi), and Rab5a (vesicles). Each of FIGS. 33-36 shows the levels of different proteins present in each fraction, which are compared with the presence of marker proteins; calnexin for the endoplasmic reticulum (ER), γ-adaptin for the Golgi (G), and Rab5a for vesicles/endosomes (V). FIGS. 34 and 35 also show the unfractionated levels of Rab6 and Rab1B, respectively, that were obtained prior to density gradient centrifugation. No difference in the expression of these 3 marker proteins was observed between the control and drug-treated cells.

B 16F1/B 16F10 Density Gradient Protocol

B16F10 cells were seeded into 175 cm2 flasks one day prior to drug application. On the subsequent day, fresh media +/−drug was applied to the cultures. 16 hours later, the cells were washed with cold Dulbecco's PBS, then harvested in ice-cold homogenization buffer: 130 mM KCl, 25 mM NaCl, 1 mM EGTA, 25 mM Tris pH7.4, plus 15 ul protease inhibitor per 5 mL buffer. 1 mL of buffer was used per flask, and the cells were scrapped off into 14 mL round-bottom culture tubes and kept on ice. The harvested cells were then homogenized with a tissue homogenizer (Polytron PT10/35), transferred into 2 mL centrifuge tubes, and spun at 1,000 rpm for 8 min at 4° C. The supernatant was collected and placed on top of a 30% to 2.5% iodixanol (Optiprep) gradient, previously prepared with homogenization buffer and kept cold. 16×100 mm ultracentrifuge tubes were used, and a Sorval OTD50B Ultracentrifuge with an AH-627 rotor, spinning the samples at 27,000 rpm for 1 hr. 1 mL samples were carefully removed from the top of the gradient, then diluted with a 2× sample buffer for Western Blot analysis (16 ul loaded per lane). NOTE: Throughout this protocol, samples were kept on ice as much as possible.

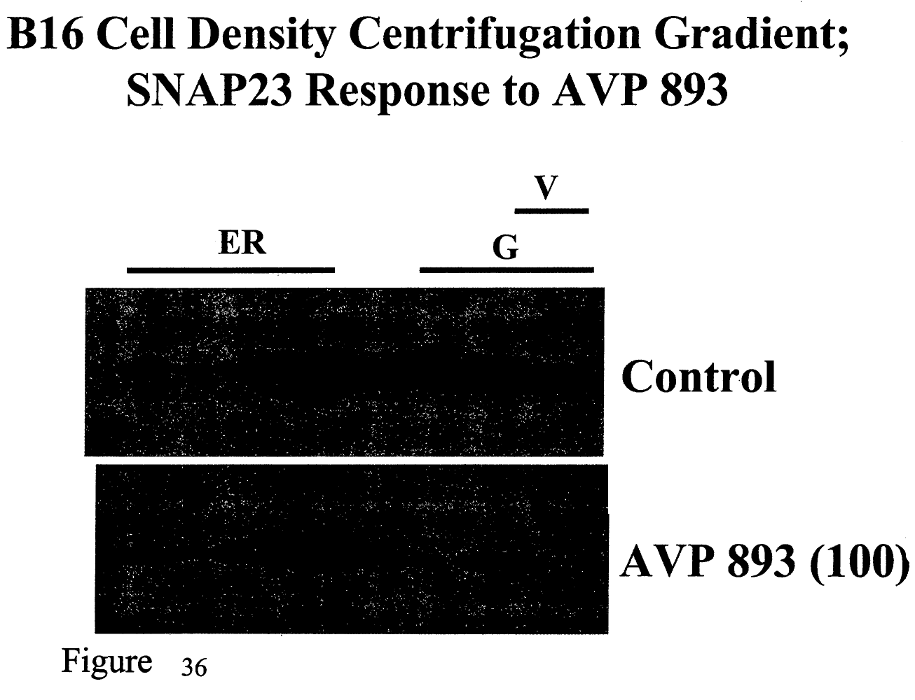

Although AVP 893-treated cells expressed much less GS28, its distribution was not significantly altered (FIG. 33). Nicastrin was distributed much more diffusely, and expressed predominantly as the partially glycosylated form in all fractions of lysates from drug treated cells (vs control cells). Rab 1b and Rab 6 expression were also tested. The results are illustrated in FIGS. 34 and 35, respectively. Although neither protein was quantitatively reduced in unfractionated lysates (in contrast to GS28), both Rab 1b and Rab 6 were retained in the ER at the expense of the golgi compartment. Similar results were noted for Rab1a (not shown). Rab 6 also appeared to localize in the vesicles suggesting a possibility that vesicle fusion with either the ER (retrograde) or golgi (anterograde) was inhibited by AVP 893. SNAP23, a SNARE protein located predominantly in a post-golgi compartment, experienced a similar shift to the ER (FIG. 36). In this case, however, SNAP23 is expressed in the ER as a cargo protein, passing from the ER to the golgi in transit to its peripheral compartment.

Comparison with Brefeldin A

Of the few chemical compounds known to affect the intracellular trafficking of proteins, the two most studied are Monensin and Brefeldin A. Monensin is a sodium ionophore that shares some of the effects noted for the AVP compounds (e.g., cytokine inhibition). However, because it acts in a post-golgi compartment, there are qualitative inconsistencies in their activity that clearly demonstrate that the compounds act differently. Brefeldin A, however, blocks movement of proteins from the ER to the golgi and shares many of the effects observed for AVP 893, including cytokine production/release and tumor cell proliferation. The mechanism of Brefeldin A is reasonably well mapped out and involves golgi disruption through inhibition of GDP-GTP transfer on Arfl, a GTPase responsible for activating budding of retrograde COPII vesicles from the golgi to the ER. However, although Arfl is primarily located in the ER-golgi region, it is also found in other compartments and appears to have more broad effects than just the ER-golgi area.

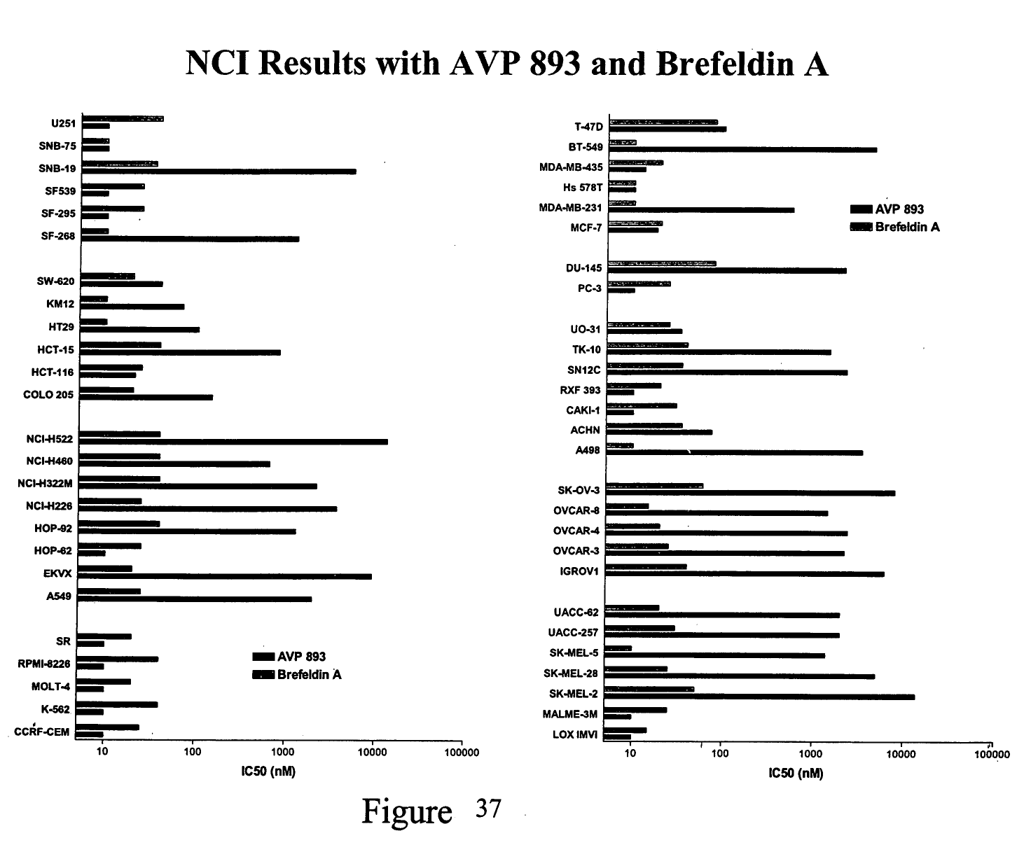

Brefeldin A was tested by the NCI for inhibition of tumor cell proliferation in the 60-cell screen. The NCI 60-cell screen was performed essentially as described for FIG. 9. Data available from the NCI database for Brefeldin A was compared with more recent AVP 893 data. Comparison of the results obtained for Brefeldin A with that of AVP 893 show that while Brefeldin A inhibits proliferation of virtually all cells at concentrations of 10 to 100 nM, AVP 893 showed considerable variation in potency (<10 nM to >10 μM) depending upon the cell line tested (FIG. 37). Several tumor cell lines were cultured in the presence of either AVP 893 or Brefeldin A for about 72 hours before assessing proliferation response by measuring total protein (SRB), as described for FIG. 9. The results of the head-to-head comparisons performed in-house also show substantial variation in the relative proliferative responses of cells to Brefeldin A and AVP 893 in vitro (TABLE 3).

TABLE 3
Inhibition of Tumor Cell Proliferation In Vitro

| | | IC50 (ng/ml) | | |
|---|---|---|---|---|
| Cell Line | NCI | Avanir | AVP 893 | Brefeldin A |
| MOLT-4 | <10 | 0.001 | 30 | 3 |
| Hs578T | <10 | 2 | <1 | 100 |
| HCC 1806 | n/a | 30, 300 | 4 | 30 |
| OVCAR3 | 1000 | 200 | | |
| NCI H-460 | 400 | >2000 | >3000 | 5 |
| SW480 | n/a | 1500 | | |

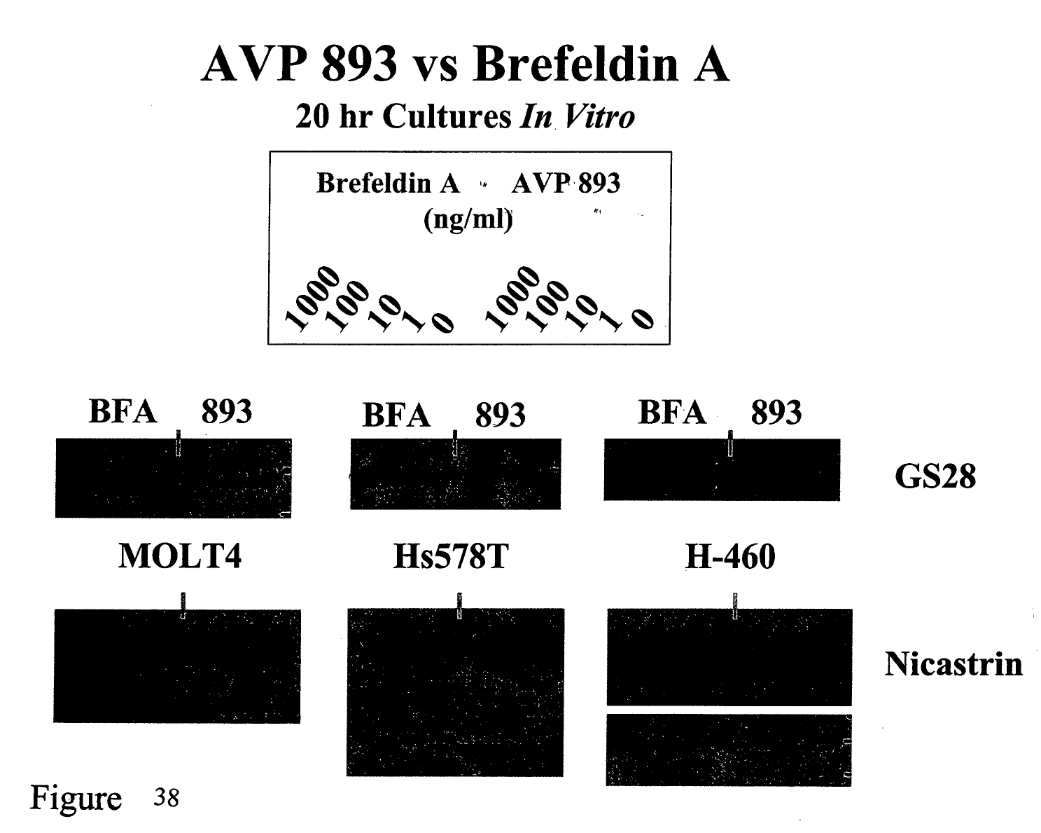

A further comparison of the compounds' effect on protein expression was carried out in the cell lines outlined in TABLE 3. As shown in FIG. 38, AVP 893 inhibited GS28 (and mature nicastrin) expression in the 2 “sensitive” cell lines at concentrations that closely paralleled their activity on proliferation. MOLT-4, Hs294T, and H460 cells were cultured overnight with either AVP 893 or Brefeldin A and harvested and prepared for Western blotting as described for FIG. 17. AVP 893 had little effect on GS28 or nicastrin in the resistant line, H-460. In contrast, Brefeldin A had variable effects on GS28 ranging from a small diminution (MOLT4, Hs578T) to a large increase in expression (H-460) at high concentrations. Moreover, the changes observed for GS28 did not parallel the IC50 of Brefeldin A for proliferation in these cell lines. Effects on nicastrin were minimal.

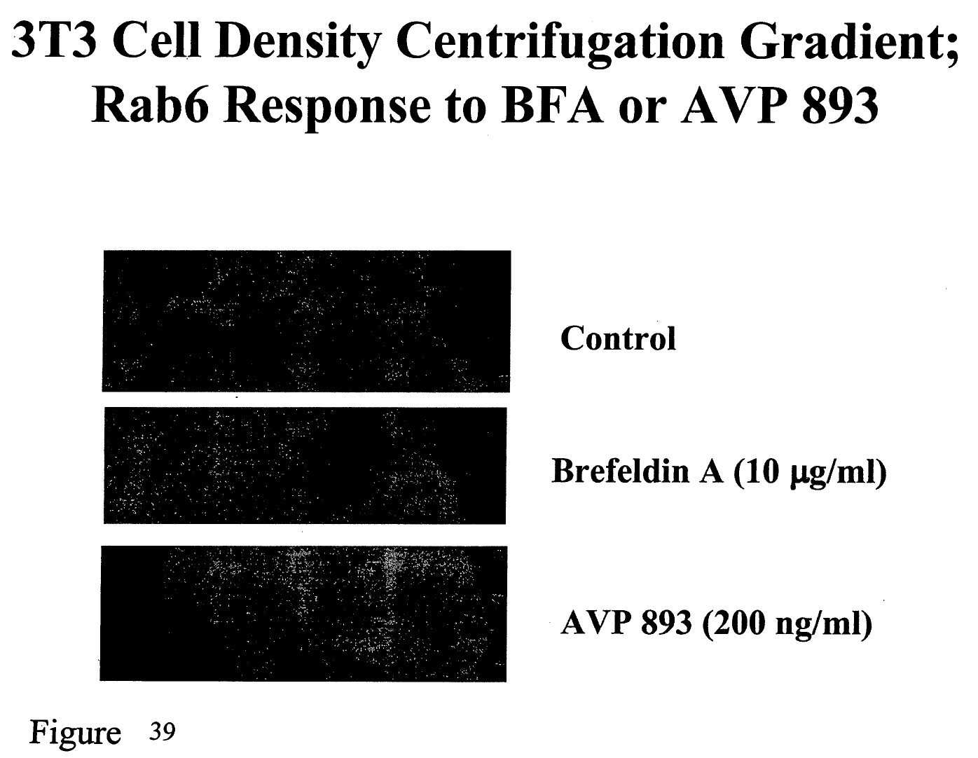

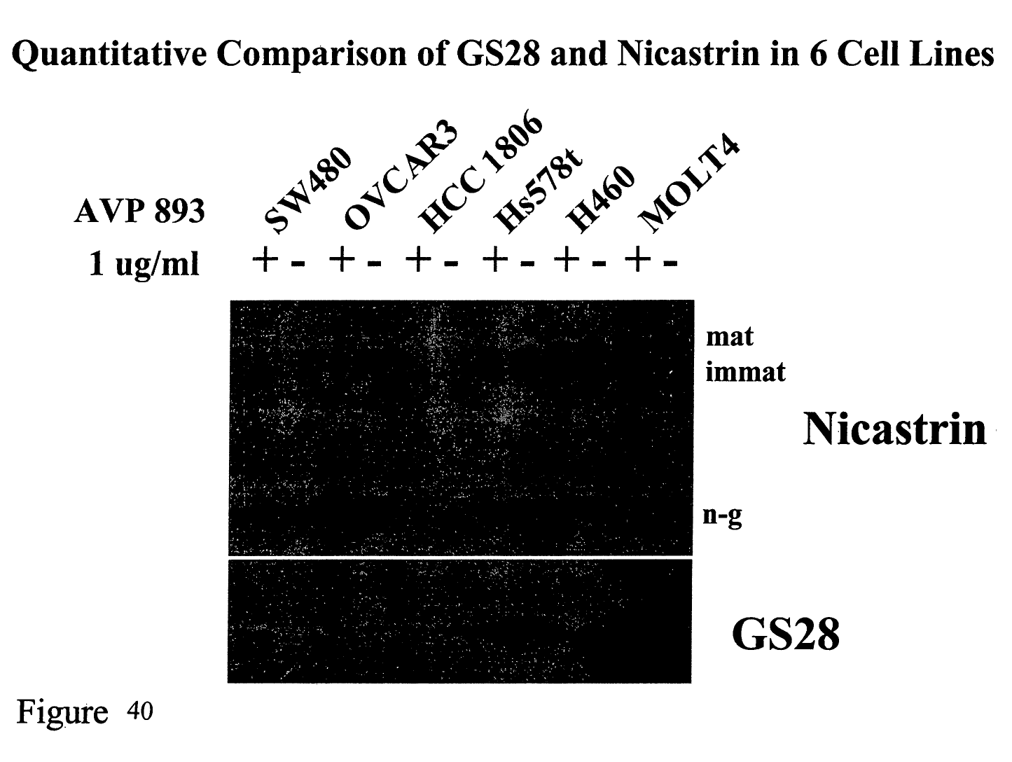

These results clearly show that Brefeldin A and AVP 893 act via different mechanisms to inhibit protein trafficking. Initial results comparing density gradient centrifugations of lysates from cells treated with either AVP 893 or Brefeldin A show that the two compounds modify the distribution of Rab 6 in a similar manner (FIG. 39). 3T3 cells were cultured, harvested, and prepared for density gradient centrifugation similar to the procedure described in FIG. 33. This supports the notion that AVP 893 is acting to inhibit protein movement through the ER-golgi. AVP compounds suppress GS28 in all non-transformed cells tested, but not all tumor cells respond in this manner (FIG. 40). Lysates from 6 cell lines that were treated with AVP 893 at 1 μg/ml for 18-20 hours were compared for their expression of Nicastrin and GS28. The same amount of total protein was loaded in each lane for Western blotting. Tumor cells undergo a variety of genetic modifications and, as such, may circumvent normal protein trafficking in order to increase its proliferative capacity. Thus, although the specific target for AVP 893 (or Brefeldin A) has not been identified, inhibition of protein trafficking through the ER-golgi is proposed as its mechanism.

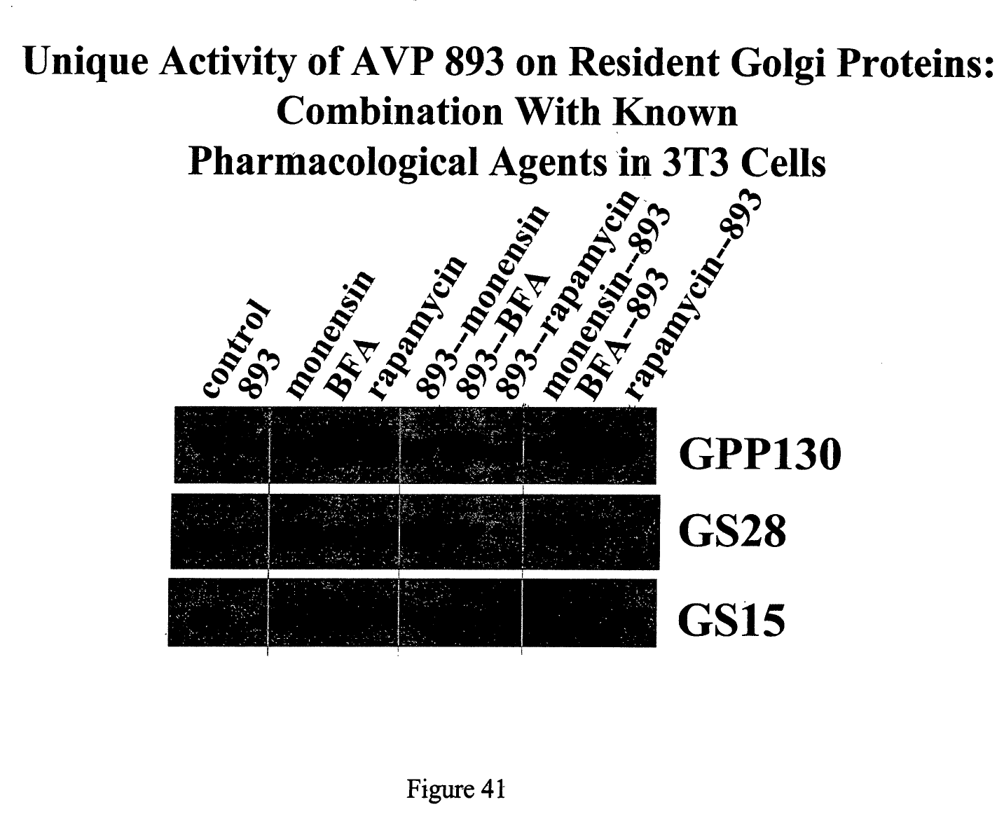

Further studies were conducted to show that AVP 893 has unique activity against resident golgi proteins, as compared to pharmacological agents known to affect the golgi. This comparison between the activity of AVP 893 and the known agents monensin, Brefeldin A, and rapamycin, helps demonstrate that AVP 893 affects resident golgi proteins in a unique fashion. For combination treatments, the first agent was added 1 hr before the second agent; 18 hour incubations followed. The doses of agents were as follows: AVP 893, 200 ng/ml; Brefeldin A, 10 mg/ml; monensin, 10 mg/ml; rapamycin, 10 nM. As shown in FIG. 41, AVP 893 decreased the expression of GS28 and GS15 more markedly than the other three agents, and its effect on GPP130 (causing expression of the lower, putative immature-form of the glycoprotein) was matched only by monensin. In addition, Brefeldin A and monensin, when combined with 893, dominated its activity, showing only a Brefeldin A or monensin-induced ‘phenotype’ of expression. Only when 893 was combined with rapamycin did the 893 ‘phenotype’ of protein expression occur. Thus, the activity of AVP 893 against resident golgi proteins was unique and distinct from the known pharmacological agents monenin, Brefeldin A, and rapamycin.

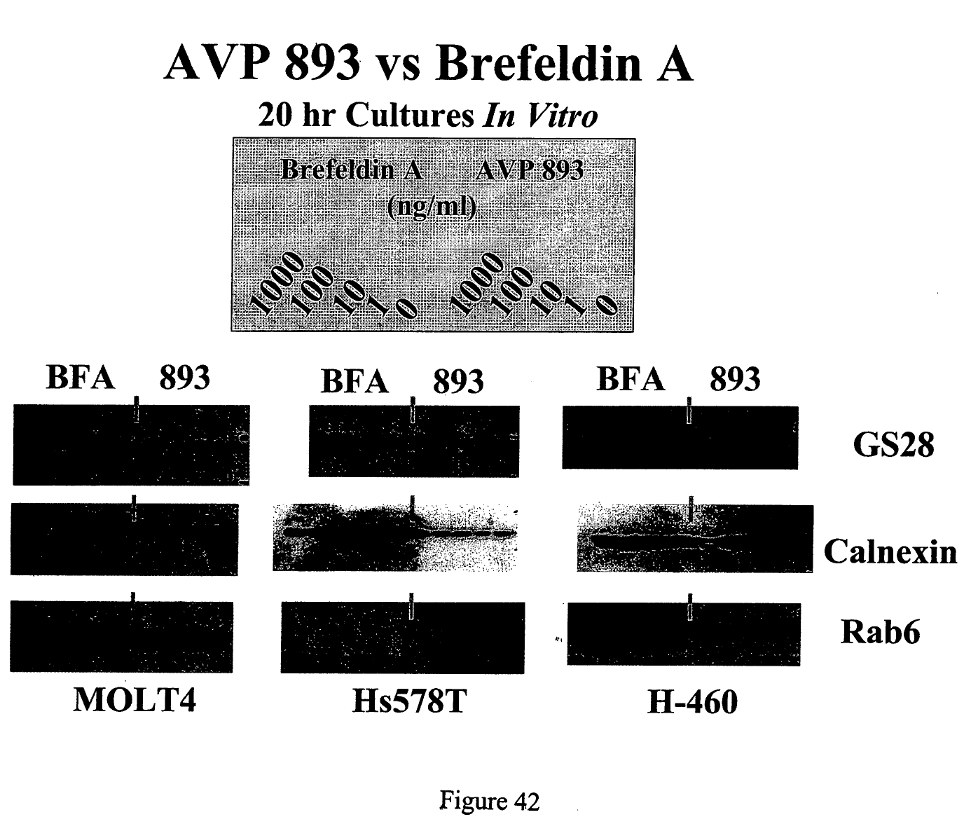

To determine whether the unique activity of AVP 893, as compared to another known pharmacological agent, Brefeldin A, the effects of increasing doses of AVP 893 and Brefeldin A on protein expression were compared in multiple cell lines. AVP 893 was shown to affect the resident golgi protein GS28 in a fashion different from Brefeldin A, across three different cell lines (FIG. 42). The effective range of AVP 893 treatment did not closely follow that of Brefeldin A. Furthermore, Rab6 expression was again shown to be largely unaffected by AVP 893, whereas Brefeldin A had varying effects on its expression, depending on the cell type. In conclusion, the unique activity of AVP 893 was present across multiple cell lines.

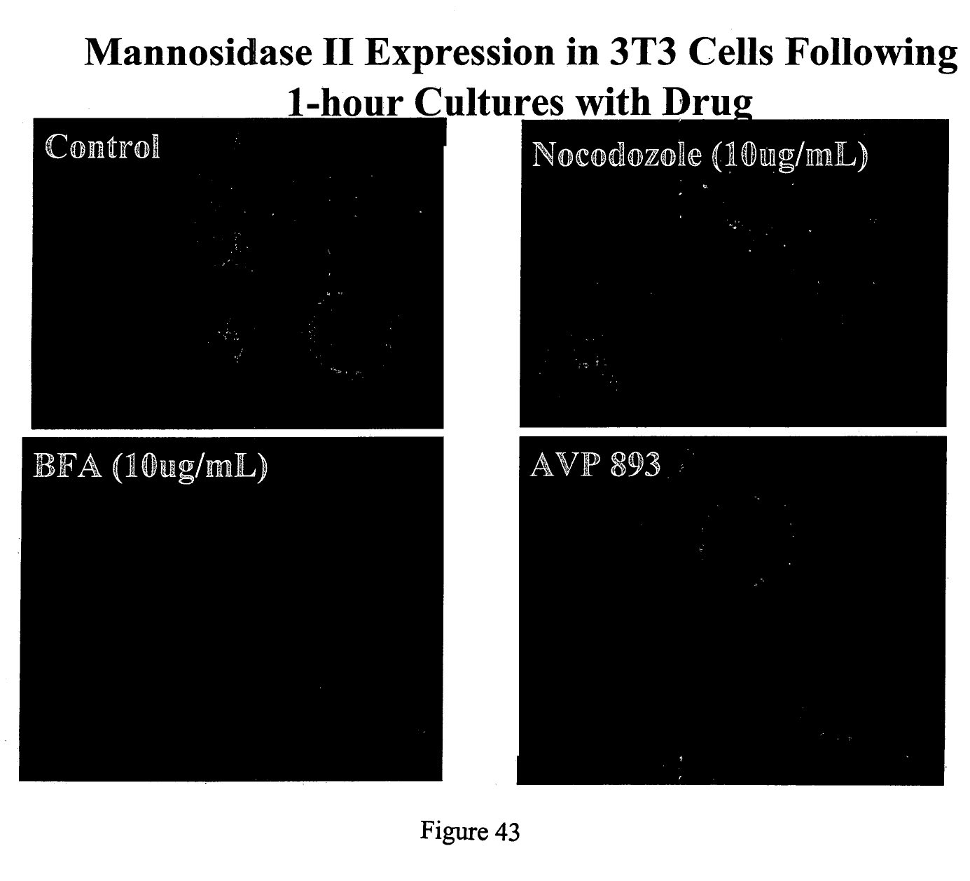

Additional evidence that AVP 893 has unique activity against resident golgi proteins (e.g. Mannosidase II), was found using both shorter durations of drug exposure and immunocytochemistry instead of western blot analysis (FIG. 43). This experiment showed that 1 hr of treatment of Brefeldin A and nocodozole disrupted the normal pattern of staining of Mannosidase II. The crescent-shaped golgi labeling was either completely dispersed, in the case of Brefeldin A, or spread into a myriad of small, punctate fragments, in the case of nocodozole. However, 1 hr of AVP 893 exposure had no apparent effect in this experiment, and certainly not any perturbation of Mannosidase II localization or expression levels. In conclusion, the results shown in FIG. 43 provide further evidence that AVP 893 acts in a unique fashion against resident golgi proteins.

Other Biological Effects Predicted by Inhibition of Protein Trafficking

Demonstration of an ER-to-golgi trafficking inhibition provides a clear explanation of the observed effects of the AVP compounds. The classical ER-golgi pathway is the preferred transportation/maturation path of most intracellular proteins, including IgE, many membrane receptors, and many cytokines. One exception to the latter is 1L-1, which by-passes the ER-golgi by the “non-classical” secretion pathway. Although AVP 13358 inhibits secretion of most cytokines, it does not affect IL-1 levels in vitro.

The proposed mechanism of the AVP compounds on intracellular protein transit also allows certain predictions as to other effects and non-effects that these compounds might share. For example, inhibition of vesicle fusion or budding between the ER and golgi should not affect exocytosis as would be expected of a post-golgi active compound such as Monensin. AVP 893 has minimal effects on the expression of proteins involved in exocytosis, particularly VAMP, SNAP23 (non-neuronal cells), and SNAP25 (neuronal cells). Accordingly, the compound does not affect the release of norepinephrine or the re-uptake of dopamine in PC12 pheochromocytoma cells (not shown). Moreover, the AVP 893 analog, AVP 13358, does not inhibit degranulation of rat basophilic leukemia (RBL) cells when induced with PMA/ionomycin or IgE-antigen complexes (not shown).

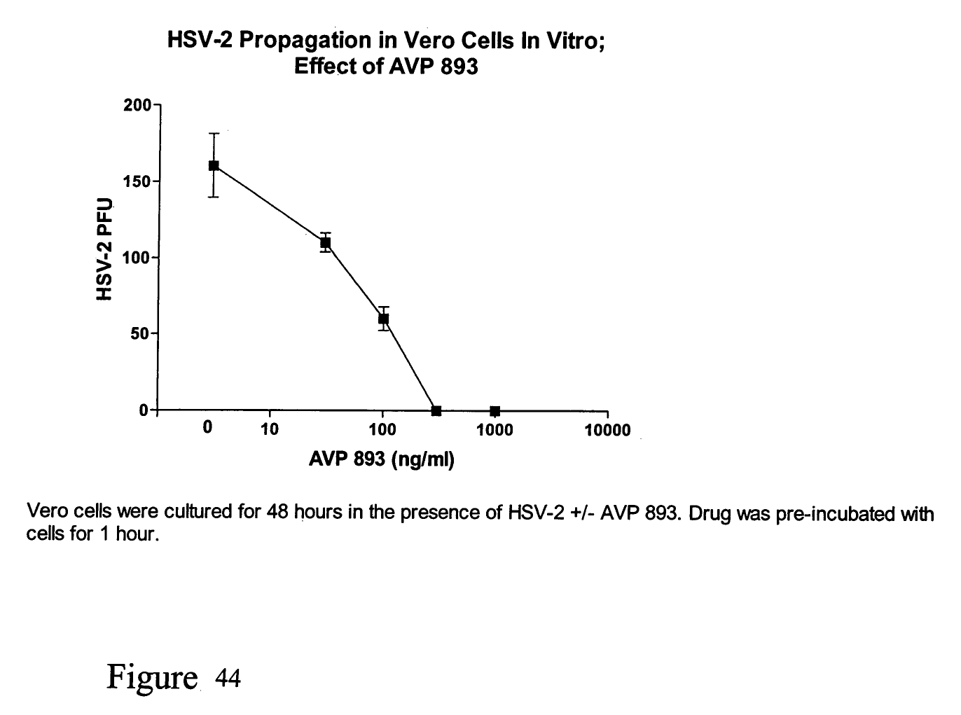

An important potential consequence of blocking normal vesicle movement between the ER and golgi is the inhibition of viral protein maturation and intracellular propagation. Most viruses rely on the classical ER-to-golgi pathway for assimilating its proteins and, ultimately, infectivity. Brefeldin A causes the accumulation of viral proteins in the ER-golgi. The capacity of AVP 893 to inhibit viral propagation was tested in vitro by infecting Vero cells with HSV-2 and observing the effect of increasing concentrations of drug (FIG. 44). Vero cells (1 million/ml) were cultured overnight and inoculated with about 150 PFU of live type 2 Herpes Virus (HSV-2, ATCC) about 1 hour after addition of AVP 893. After 48 hours, media was removed and the cells washed with saline and stained with Biological Plaque Stain for 20 min. One ml of water was added and the liquid removed before quantifying virus by enumerating PFU. AVP 893 suppressed plaque formation at all concentrations tested with a total block occurring at 300 ng/ml. Moreover, the steep concentration-response curve suggests a non-competitive inhibition, as would be expected of a drug that acts on the host cell rather than the virus.

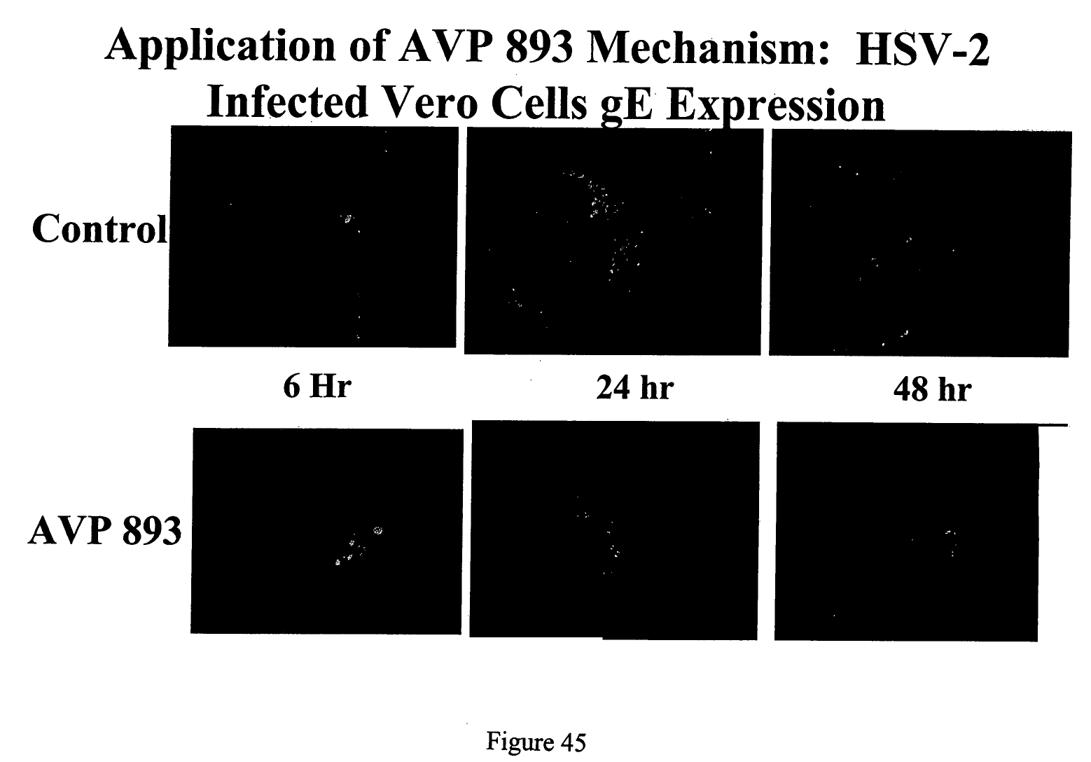

The effect of AVP 893 on the spread of viral infection was further investigated. AVP 893 (at 300 ng/ml) was applied 16 hr prior to virus inoculation. Time points shown in FIG. 45 represent the hours after virus inoculation. Having demonstrated that AVP 893 acts on the expression and localization of resident golgi proteins, the next series of experiments examined the effect of AVP 893 on HSV, a virus that utilizes the golgi in its life-cycle. In addition to the immunocytochemistry work shown in FIG. 45, extensive in vitro plaque assays were performed on HSV-1 and -2, as well as other families of virus that use the golgi in their life cycle (see Table 2).

We determined whether viral particles (as visualized in FIG. 45 by labeling HSV-2 glycoprotein E) spread beyond the initial site of infection in the presence of AVP 893. HSV-2-infected cultures were treated with AVP 893. The results demonstrate that beyond the initial site of infection, little to no labeling was found in surrounding cells. Other HSV proteins including gB, gD, and the capsid protein ICP5 were also examined with similar results (data not shown). In conclusion, AVP 893 blocked the spread of HSV-2 virus particles (or at least blocks the spread of infectious particles). Furthermore, AVP 893 didn't appear to stop the initial infection of the culture, only the subsequent spread of the virus. These results provide proof-of-concept that AVP 893, through its effect on resident golgi proteins, may be inhibiting the spread of virus particles that utilize the golgi in their lifecycle, as HSV-2 does.

In addition to affecting the expression of HSV proteins, AVP 893 was demonstrated to exert antiviral activity against other viral families. Representative viruses from families likely to utilize the golgi were tested. As shown in TABLE 4, the spread of many other viral families were inhibited by AVP 893 in vitro. In addition, a guinea pig topical HSV model has shown that AVP 893 may inhibit viral activity in vivo. (data not shown).

TABLE 4
Summary of Viral Families and the Effect of AVP 893

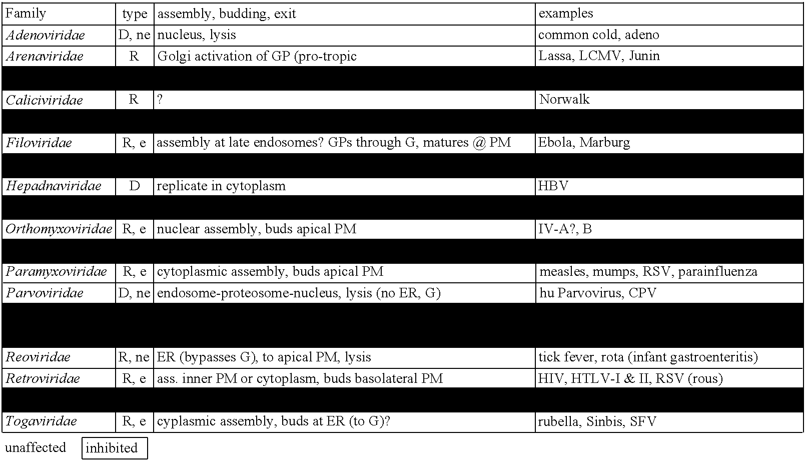

Inhibitors of Intracellular Protein Trafficking.

Preferred aspects of the described invention encompass chemical compounds of at least seventeen (17) structural classes (TABLE 5). Compounds representing all of these series inhibit IgE response and cell proliferation in vitro at similar concentrations where ER-to-golgi protein trafficking is inhibited. The latter is evidenced by inhibition of GS28 expression in non-transformed cells (FIG. 45).

TABLE 5

| | | | |
|---|---|---|---|
| 350 | 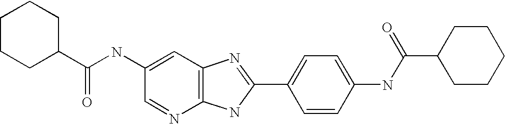 | 26350 | 40 |
| 359 | 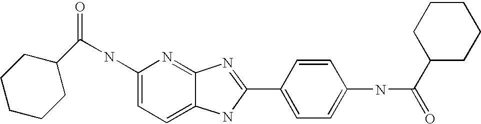 | 26359 | 25 |
| | 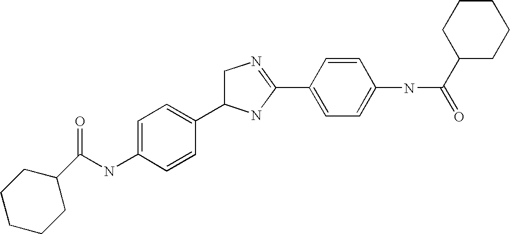 | | |
| 359 | 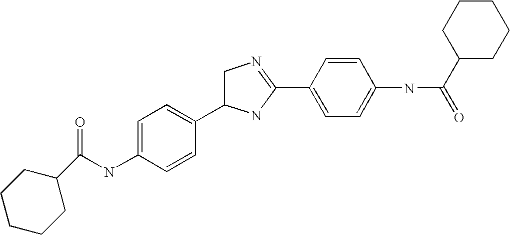 | 26359 | 25 |
| 405 | 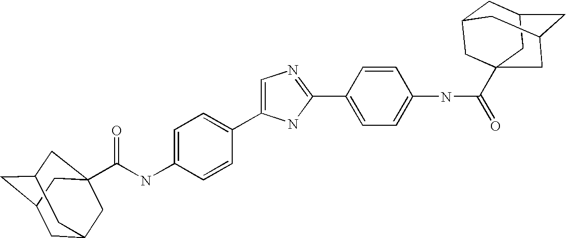 | 26405 26428 | 14 30 |
| 410 | 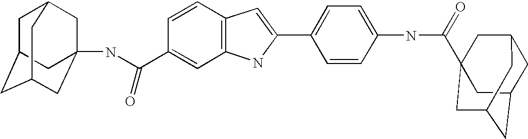 | 26410 26411 26412 26472 | 40 40 12 12 |
| 438 | 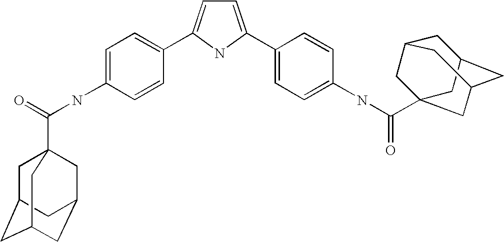 | 26438 26489 | 100 |
| 449 | 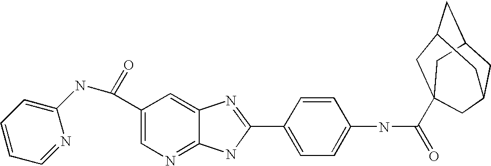 | 26449 | 40 |
| 465 | 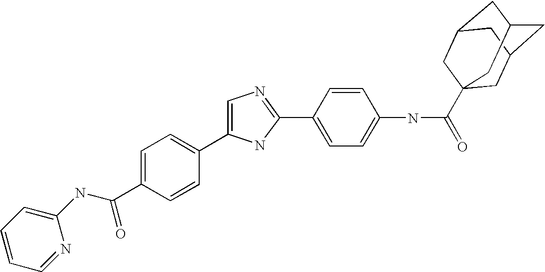 | 26465 | 5 |
| 135 | 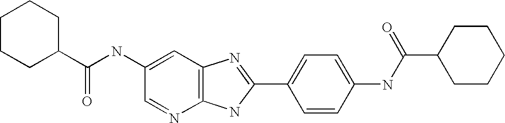 | 26135 | 60 |
| 350 | 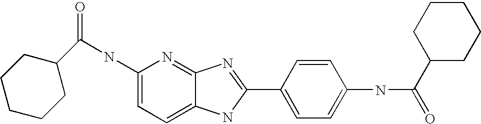 | 26350 | 40 |
| 359 | 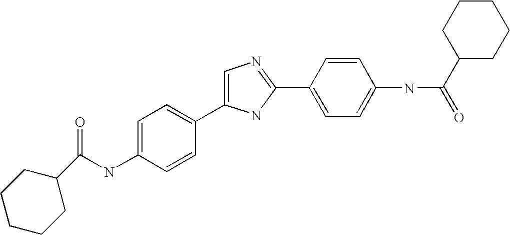 | 26359 | 25 |
| 359 | 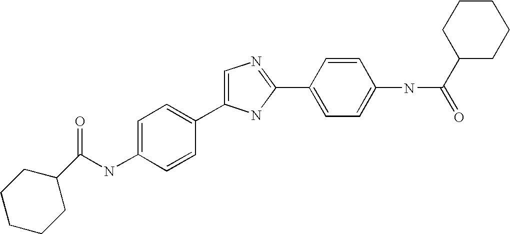 | 26359 | 25 |
| 405 | 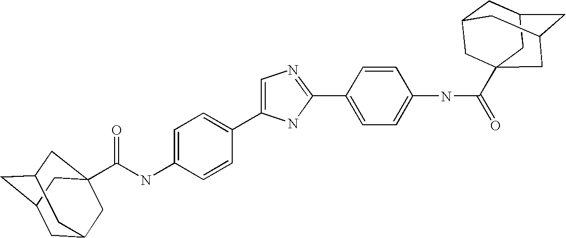 | 26405 26428 | 14 30 |
| 410 | 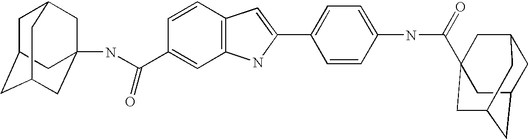 | 28410 26411 26412 26472 | 40 40 12 12 |
| 438 | 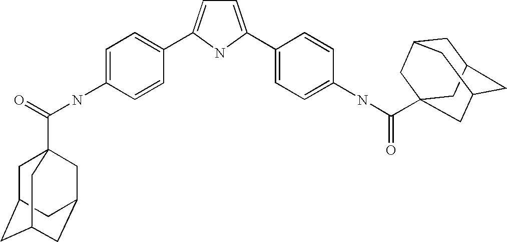 | 26438 26489 | 100 |
| 449 | 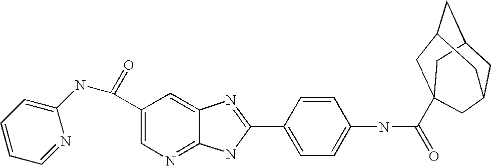 | 26449 | 40 |
| 465 | 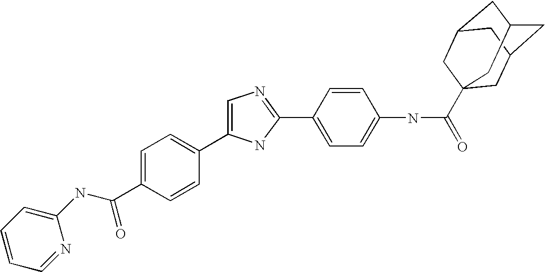 | 26465 | 5 |

Preferred aspects of the present invention relate to a novel mechanism for selectively modulating protein trafficking, which impacts numerous biological processes, including allergy, cell proliferation, and viral replication. More particularly, aspects of the present invention relate to the identification and characterization of compounds that regulate this mechanism and thereby modulate the biological processes. As described herein, both the t-SNARE protein, GS28, which is involved in the docking and fusion of vesicles in the golgi and the intermediate compartment (IC, located between the ER and golgi) and nicastrin, which participates in the intramembrane cleavage of proteins that translocate into the nucleus and act as transcription factors, were found to be affected by compounds that exhibit a wide range of biological activities. It was further elucidated that treatment with these compounds blocked nicastrin maturation such that the immature, partially glycosylated form of nicastrin accumulates at the expense of the fully glycosylated active moiety. Nicastrin normally passes through the ER where it is partially glycosylated and then to the golgi where glycosylation and sialation is completed. Thus, changes in nicastrin state seem to correlate with its intracellular compartment as it moves through the cell. By suppressing the maturation of nicastrin, these compounds may prevent the ER-to-golgi trafficking of nicastrin. The prevention of nicastrin trafficking may be due to the diminished expression of GS28 in the presence of drug.

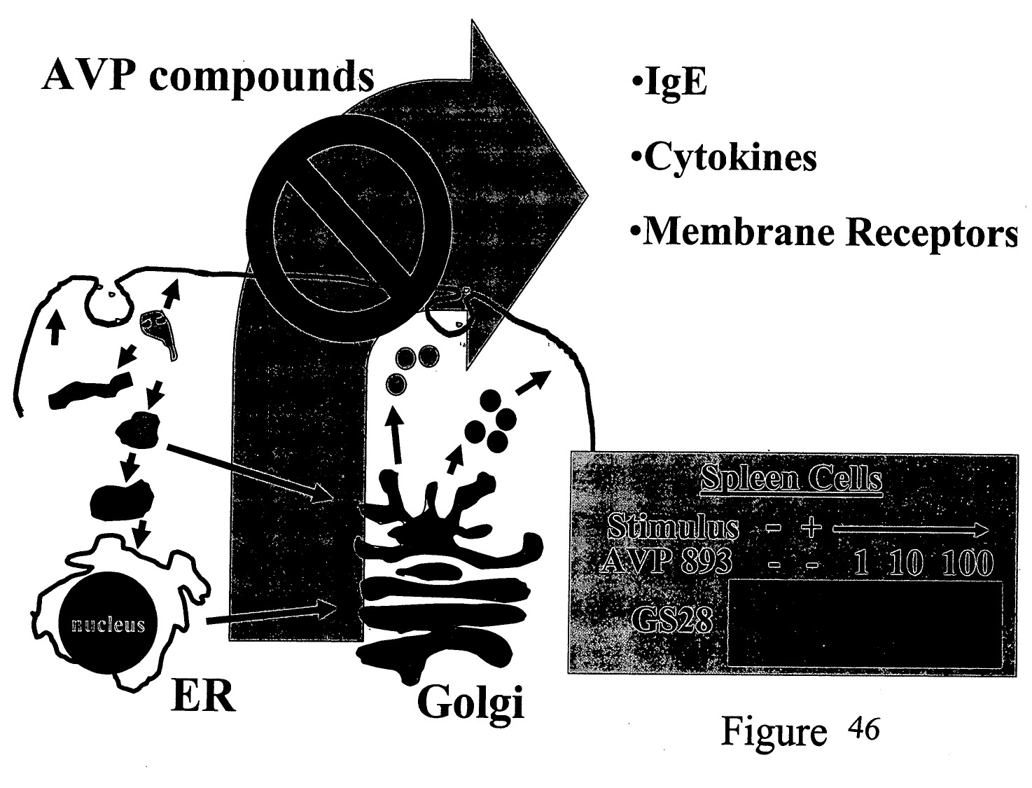

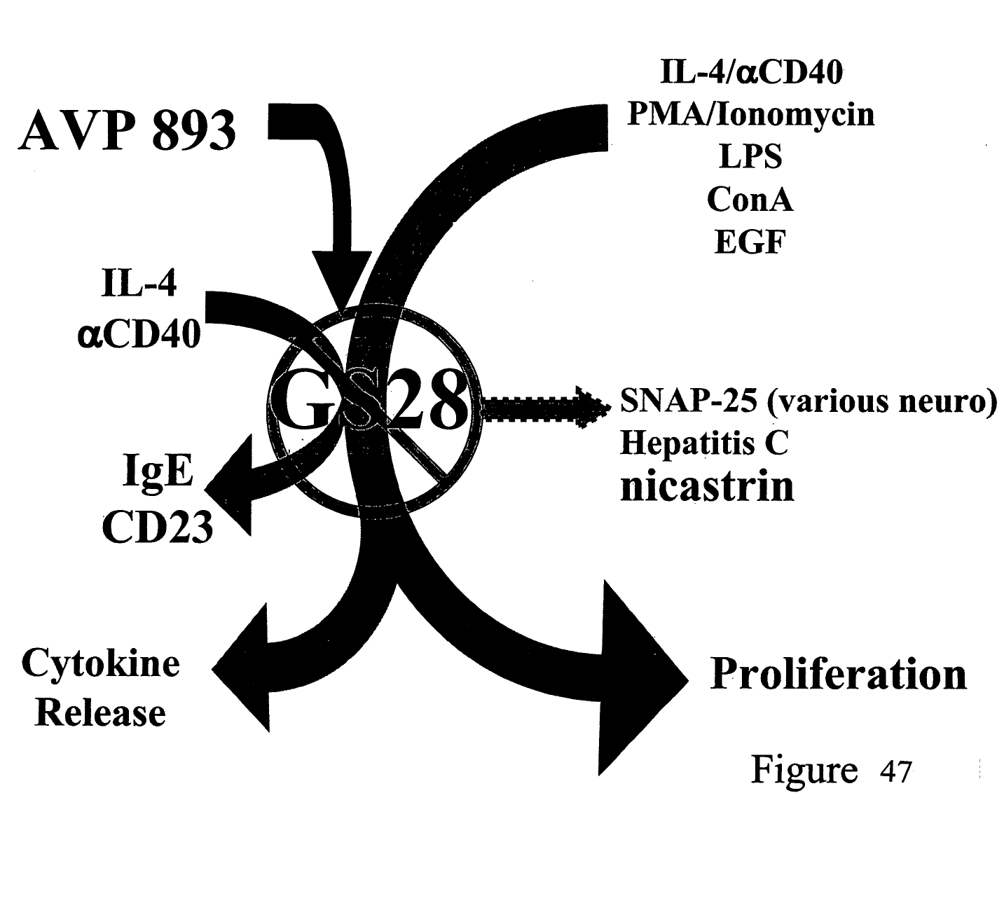

The above description of preferred embodiments of the present invention is not intended to be limiting on the scope of the invention. Indeed, Jung et al. (Electrphoresis (2000) 21:3369-3377) indicate that there are 157 resident proteins (SWISS—PROT database; Table 1) associated with the ER and golgi apparatus. Taylor et al. (Electrophoresis (1997) 18:643-654) reported 173 proteins in rat hepatocyte golgi. Thus, there may be many other ER/golgi protein targets, besides GS15, GS28, nicastrin and Rabs (shown herein to be suppressed by the AVP compounds), that influence protein trafficking in disease states (inter alia allergy, cancer, viral infection), via the same or redundant pathways described above (See e.g., FIG. 46). Accordingly, whereas pharmacologic suppression of GS28 levels, for example, has been identified by the inventors as one preferred means for selectively regulating protein trafficking that is necessary for proliferative (or viral replicative) cellular responses, modulation of other ER/golgi-associated proteins that act in concert with GS28 or which supplement or enhance the effects of GS28 may represent other preferred means for treating proliferative/replicative disorders (as shown in schematic form in FIGS. 46 and 47). Alternatively, combination therapies with other agents that target other ER/golgi proteins such that suppression of the pathologic trafficking response is enhanced, represent another embodiment within the scope of the present invention.

A compelling aspect of the preferred embodiments of the present invention is that redundant protein trafficking pathways, and the proteins involved therein, operate to allow cells to carry out their normal (or “good”) protein trafficking needs, despite selectively suppressing the “bad” trafficking associated with cells implicated in the disease condition (e.g., transformed, infected, etc.). Accordingly, the inventors have found that toxicity is minimized (in contrast to treatment regimens employing Brefeldin A) using the selective pharmacologic therapies disclosed herein.

Those skilled in the art will recognize or be able to ascertain, using no more than routine experimentation, many equivalents of the specific embodiments of the invention described therein. Such equivalents are intended to be encompassed by the following claims.

1. A method for selectively inhibiting eukaryotic cell proliferation associated with a disease condition, comprising administering an amount of a composition sufficient to suppress expression of at least one ER/golgi resident protein associated with proliferation-dependent protein trafficking between the ER and golgi, such that the cell proliferation associated with the disease condition is inhibited.

2. The method of claim 1, wherein said at least one ER/golgi resident protein is selected from the group consisting of GS 15, GS28, nicastrin and a Rab.

3. The method of claim 1, wherein said at least one ER/golgi resident protein is GS28.

4. The method of claim 1, wherein said composition comprises a compound selected from the group consisting of: 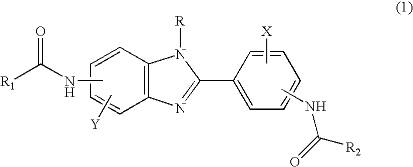
wherein X and Y are independently selected from the group consisting of H, alkyl, alkoxy, aryl, substituted aryl, hydroxy, halogen, amino, alkylamino, nitro, cyano, CF₃, OCF₃, CONH₂, CONHR and NHCOR₁;
wherein R is selected from the group consisting of H, CH₃, C₂H₅, C₃H₇, C₄H₉, CH₂Ph, and CH₂C₆H₄—F(p-); and
wherein R1 and R2 are independently selected from the group consisting of H, aryl, substituted aryl, cycloaryl substituted cycloaryl, multi-ring clycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like, wherein at least one of R1 and R2 are aromatic groups, 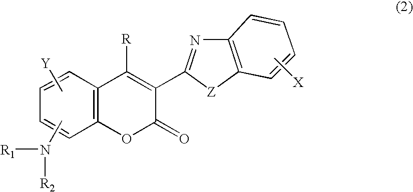
wherein X and Y are selected independently from the group consisting of alkyl, alkoxy, aryl, substituted aryl, heteroaryl, substituted heteroaryl, hydroxy, halogen, NO₂, CF₃, OCF₃, NH₂, NHR₃, NR₃R₄ and CN;
wherein Z is selected from the group consisting of O, S, NH, and N—R′; wherein R′ is further selected from the group consisting of H, alkyl, aminoalkyl, and dialkylaminoalkyl;
wherein R is selected from the group consisting of H, alkyl, halogen, alkoxy, CF3 and OCF3; and
R1 and R2 are independently selected from the group consisting of H, alkyl, aminoalkyl, dialkylaminoalkyl, hydoxyalkyl, alkoxyalkyl, cycloalkyl, oxacycloalkyl and thiocycloalkyl, 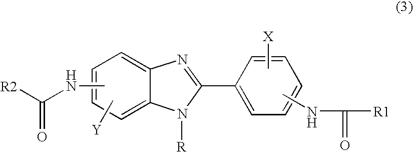
wherein X and Y are independently selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
wherein R is selected from the group consisting of H, CH3, C2H5, C3H7, C4H9, CH2Ph, and CH2C6H4-F(p-);
wherein R1 and R2 are independently selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, wherein said substitutions are not heterocyclic rings; and
wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, and substituted adamantyl are selected from the group consisting of alkyl, aryl, CF3, CH3, OCH3, OH, CN, COOR5, and COOH, 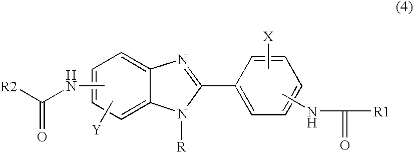
wherein X and Y are independently selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
wherein R is selected from the group consisting of H, CH3, C2H5, C3H7, C4H9, CH2Ph, and CH2C6H4-F(p-);
wherein R1 and R2 are independently selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, heterocyclic rings, and substituted heterocyclic rings;
wherein R1 and R2 cannot both be methyl groups;
wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, substituted adamantyl and substituted heterocyclic rings are selected from the group consisting of alkyl, acyl, aryl, CF3, CH3, OCH3, OH, CN, COOR5, COOH, COCF3, and heterocyclic rings; and
wherein at least one of R1, R2 or said substituents is a heterocyclic ring, 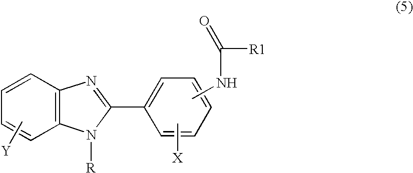
wherein X is selected from the group consisting of mono, di, tri, and tetra substituted H, alkyl, alkoxy, aryl, substituted aryl, hydroxy, halogen, amino, alkylamino, nitro, cyano, CF3, OCF3, CONH2, CONHR, and NHCOR1;
wherein R is selected from the group consisting of H, CH3, C2H5, C3H7, C4H9, CH2Ph, and CH2CH4-F(p-);
wherein Y is selected from the group consisting of mono, di, tri, and tetra substituted H, alkyl, alkoxy, aryl, benzo, substituted aryl, hydroxy, halogen, amino, alkylamino, nitro, cyano, CF3, OCF3, COPh, COOCH3, CONH2, CONHR, NHCONHR1, and NHCOR1;
wherein R1 is selected from the group consisting of alkyl, substituted alkyl, cycloalkyl, substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalkenyl, adamantyl, substituted adamantyl, heterocyclic rings containing one or more heteroatoms, and substituted heterocyclic rings; and
wherein the substituents on said substituted alkyl, substituted cycloalkyl, substituted cyclopropyl, substituted cyclobutyl, substituted cyclopentyl, substituted cyclohexyl, substituted cycloheptyl, substituted bicycloalkenyl, substituted adamantyl, and substituted heterocyclic rings are selected from the group consisting of alkyl, aryl, CF3, CH3, OCH3, OH, CN, COOR, COOH, and heterocyclic rings, 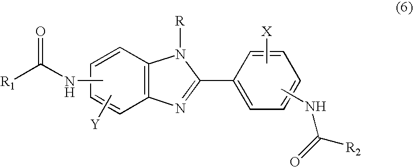
wherein X and Y are independently selected from the group consisting of H, alkyl, alkoxy, aryl, substituted aryl, hydroxy, halogen, amino, alkylamino, nitro, cyano, CF3, OCF3, CONH2, CONHR and NHCOR1;
wherein R is selected from the group consisting of H, CH3, C2H5, C3H7, C4H9, CH2Ph, CH2C6H4-F(p-); and
wherein R1 and R2 are independently selected from the group consisting of H, aryl, substituted aryl, cycloaryl substituted cycloaryl, multi-ring clycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like, wherein at least one of R1 and R2 are aromatic groups, 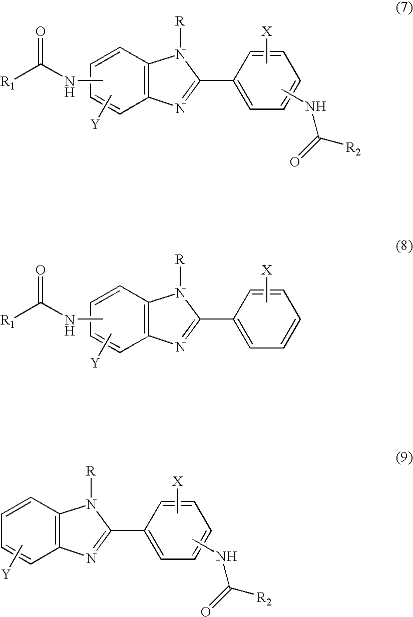
wherein X and Y are independently selected from the group consisting of H, alkyl, alkoxy, aryl, substituted aryl, hydroxy, halogen, amino, alkylamino, nitro, cyano, CF₃, OCF₃. CONH₂, CONHR and NHCOR₁;
wherein X and Y are independently selected from the group consisting of H, alkyl, alkoxy, aryl, substituted aryl, hydroxy, halogen, amino, alkylamino, nitro, cyano, CF3, OCF3. CONH2, CONHR and NHCOR1;
wherein R is selected from the group consisting of H, CH3, C2H5, C3H7, C4H9, CH2Ph, CH2C6H4-F(p-), COCH3, CO2CH2CH3, aminoalkyl and dialkylaminoalkyl; and
wherein R1 and R2 are independently selected from the group consisting of H, aryl, heteroaryl, thiophene, pyridyl, thiazolyl, isoxazolyl, oxazolyl, pyrimidinyl, substituted aryl, substituted heteroaryl, substituted thiophene, substituted pyridyl, substituted thiazolyl, substituted isoxazolyl, substituted oxazolyl, cycloaryl, cycloheteroaryl, quinolinyl, isoquinolinyl, substituted cycloaryl, substituted cycloheteroaryl, substituted quinolinyl, substituted isoqunolinyl, multi-ring cycloaryl, multi-ring cycloheteroaryl, benzyl, heteroaryl-methyl, substituted benzyl, substituted heteroaryl-methyl alkyl, dialkylaminoalkyl, cycloalkyl, cycloalkyl containing 1-3 heteroatoms, substituted cycloalkyl, substitute cycloalkyl containing 1-3 heteroatoms, multi-ring cycloalkyl, multiring cycloalkyl containing 1-3 heteroatoms, fused-ring aliphatic, fused-ring aliphatic containing 1-3 heteroatoms, cyclopropyl, substituted cyclopropyl, cyclobutyl, substituted cyclobutyl, cyclopentyl, pyrrole, piperidine, substituted cyclopentyl, cyclohexyl, substituted cyclohexyl, cycloheptyl, substituted cycloheptyl, bicycloheptyl, substituted pyrrole, substituted piperidine, bicyclooctyl, bicyclononyl, substituted bicycloalkenyl, adamantyl, and substituted adamantyl, heterocyclic ring, and substituted heterocyclic ring;
wherein at least one of R1 and R2 are aromatic groups or heteroaromatic groups; and
wherein R1 and R2 cannot both be phenyl groups, 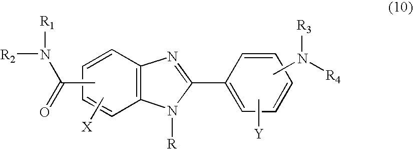
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, substituted polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein R3 and R4 are independently selected from the group consisting of H, alkyl, aryl, heteroaryl and COR′;
wherein R′ is selected from the group consisting of H, alkyl, substituted alkyl, C3-C9 cycloalkyl, substituted C3-C9 cycloalkyl, polycyclic aliphatics, substituted polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; wherein R′ is not haloalkyl;
wherein the substituent on R1, R2, and R′ is selected from the group consisting of H, halogens, polyhalogens, alkoxy group, substituted alkoxy, alkyl, substituted alkyl, dialkylaminoalkyl, hydroxyalkyl, carbonyl, OH, OCH3, COOH, OCOR′, COOR′, COR′, CN, CF3, OCF3, NO2, NR′R′, NHCOR′ and CONR′R′;
wherein X and Y are independently selected from the group consisting of H, halogens, alkoxy, substituted alkoxy, alkyl, substituted alkyl, dialkylaminoalkyl, hydroxyalkyl, OH, OCOR″, OCH3, COOH, CN, CF3, OCF3, NO2, COOR″, CHO and COR″;
wherein R″ is a C1-C8 alkyl, wherein said C1-C8 alkyl is selected from the group consisting of a straight chain, branched or cyclic alkyl; and wherein at least one of R1, R2, R3, or R4 is not H, 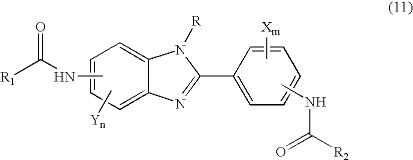
X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF3, OCF3, COR1, COOR1, CONH2, CONHR1, and NHCOR1;
n is an integer from one to three;
m is an integer from one to four;
R is selected from the group consisting of H, CH3, C2H5, C3H7, C4H9, CH2Ph, CH2C6H4-F(p-), COCH3, COCH2CH3, CH2CH2N(CH3)₂, and CH2CH2CH2N(CH3)₂; and
R1 and R2 are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom, 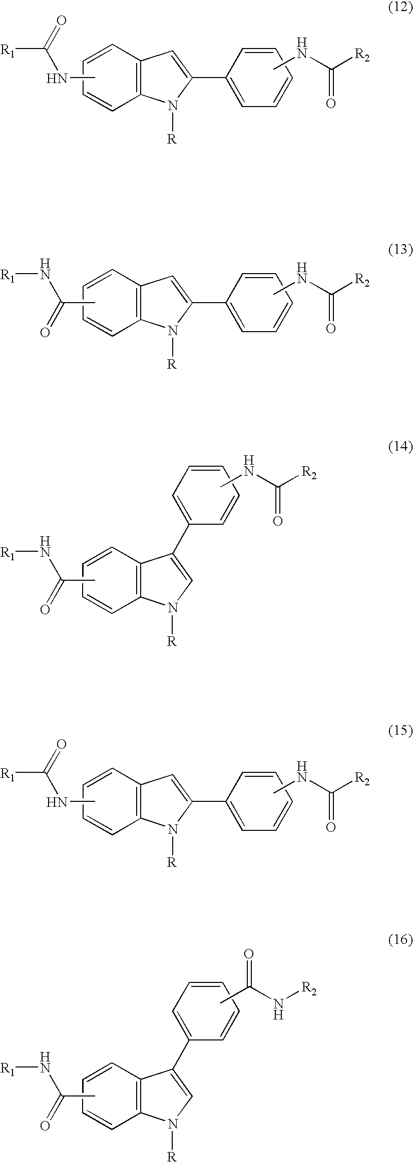 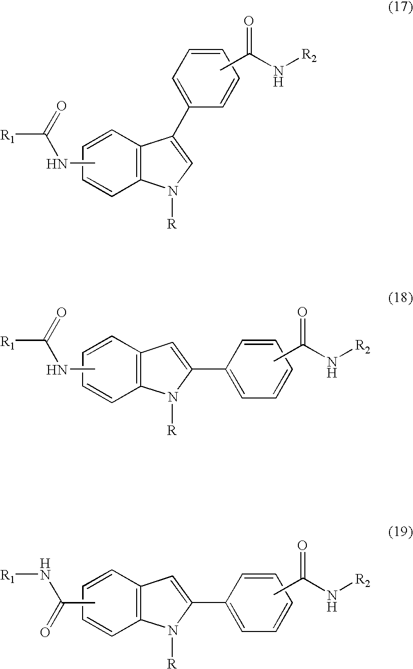
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is a C1-C8 alkyl, wherein said C1-C8 alkyl is selected from the group consisting of a straight chain, branched or cyclic alkyl, 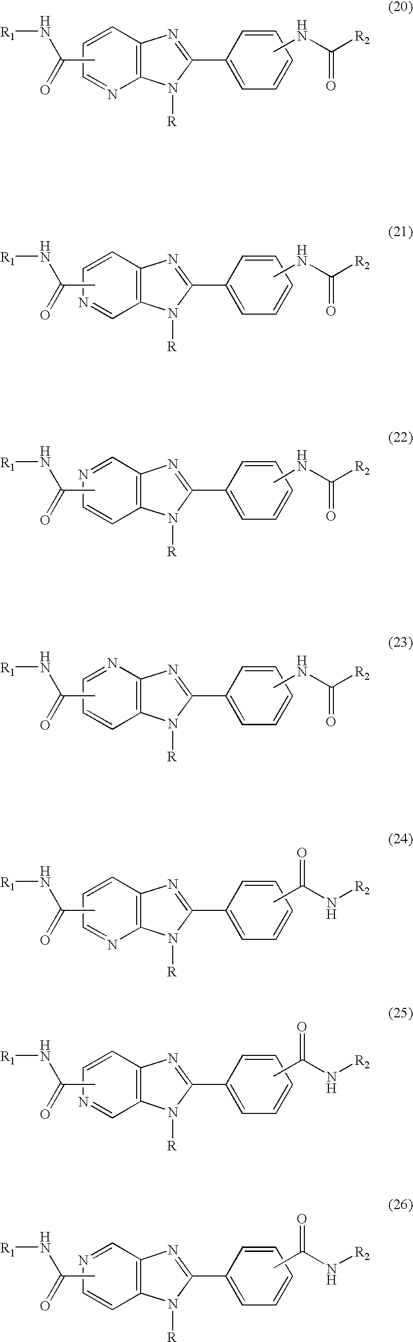 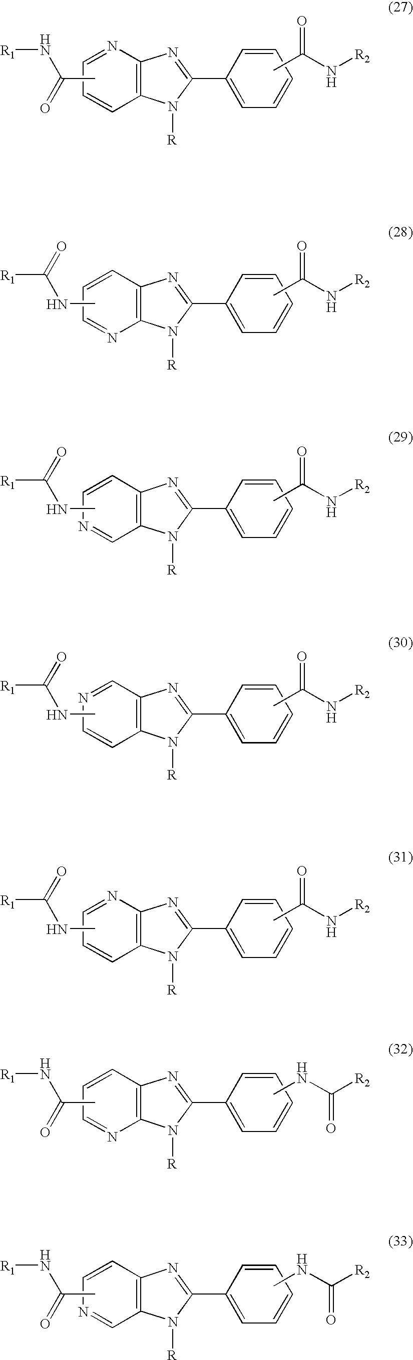 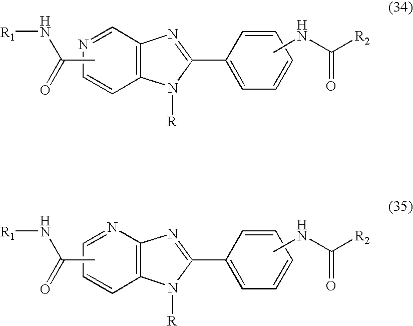
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is a C1-C8 alkyl, wherein said C1-C8 alkyl is selected from the group consisting of a straight chain, branched or cyclic alkyl, 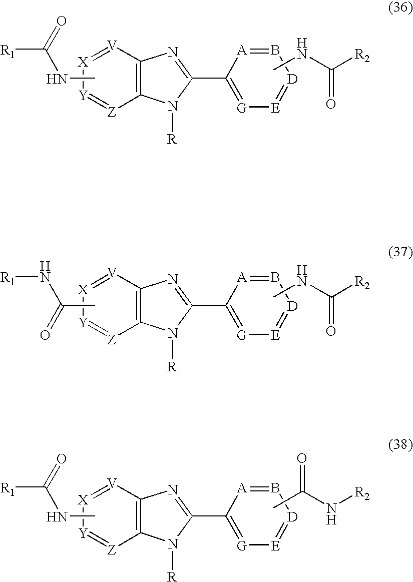
wherein A, B, D, E, G, V, X, Y, and Z are independently selected from carbon and nitrogen, with the proviso that at least one of A, B, D, E, G is nitrogen;
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur;
wherein said substituted phenyl, substituted naphthyl and substituted heteroaryl contain 1-3 substituents, wherein said substituent is selected from the group consisting of H, halogens, polyhalogens, alkoxy group, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′; and
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur, 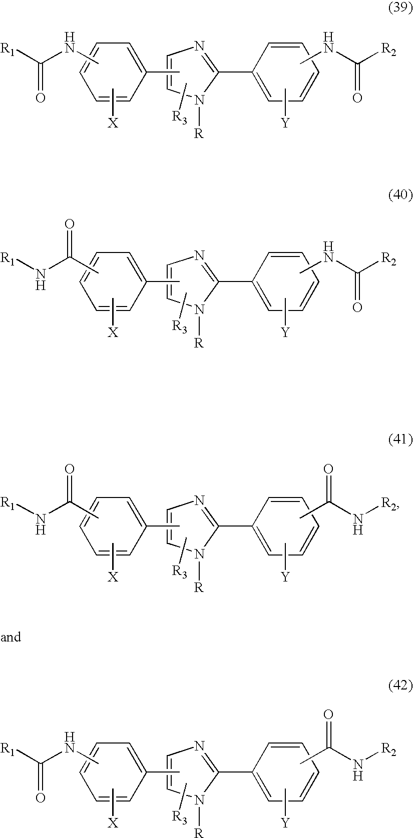
wherein R is selected from the group consisting of H, C1-C5 alkyl, benzyl, p-fluorobenzyl and di-alkylamino alkyl, wherein said C1-C5 alkyl is selected from the group consisting of a straight chain, branched or cyclic alkyl;
wherein R3, X, and Y are independently selected from the group consisting of H, halogen, alkoxy, substituted alkoxy, alkyl, substituted alkyl, dialkylaminoalkyl, hydroxyalkyl, OH, OCH3, COOH, CN, CF3, OCF3, NO2, COOR″, CHO, and COR″;
wherein R1 and R2 are independently selected from the group consisting of H, alkyl, substituted alkyl, C3-C9 cycloalkyl, substituted C3-C9 cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH3, COOH, COOR′COR′, CN, CF3, OCF3, NO2, NR′R′, NHCOR′ and CONR′R′;
wherein R′ is selected from the group consisting of H, alkyl, substituted alkyl, C3-C9 cycloalkyl, substituted C3-C9 cycloalkyl, polycyclic aliphatics, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C1-C9 alkyl, wherein said C1-C9 alkyl is selected from the group consisting of straight chain alkyl, branched alkyl, and cyclic alkyl.

5. The method of claim 1, wherein said composition comprises AVP 893.

6. A method for selectively inhibiting eukaryotic cell proliferation associated with a disease condition, comprising administering an amount of a composition comprising the compound: 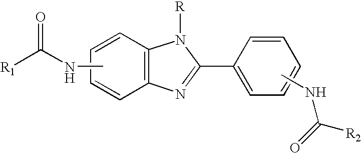
wherein R is selected from the group consisting of H, CH₃, C₂H₅, C₃H₇, C₄H₉, CH₂Ph, CH₂C₆H₄—F(p-); and
wherein R₁ and R₂ are independently selected from the group consisting of H, aryl, substituted aryl, cycloaryl substituted cycloaryl, multi-ring cycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like; and
wherein said amount is sufficient to suppress expression of at least one ER/golgi resident protein involved in proliferation-dependent protein trafficking between the ER and golgi, such that the cell proliferation associated with the disease condition is inhibited.

7. A method for selectively inhibiting eukaryotic cell proliferation associated with a disease condition, comprising administering an amount of a composition comprising the compound: 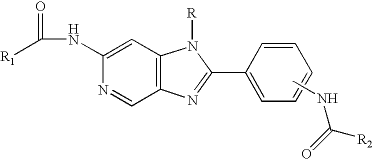
wherein R is selected from the group consisting of H, CH₃, C₂H₅, C₃H₇, C₄H₉, CH₂Ph, and CH₂C₆H₄—F(p-); and
wherein R₁ and R₂ are independently selected from the group consisting of H, aryl, substituted aryl, cycloaryl substituted cycloaryl, multi-ring cycloaryl, benzyl, substituted benzyl, alkyl, cycloalkyl substituted cycloalkyl, multi-ring cycloalkyl, fused-ring aliphatic, cyclopropyl, substituted cyclopropyl, cyclobutyl, substituted cyclobutyl, cyclopentyl, substituted cyclopentyl, cyclohexyl, substituted cyclohexyl, cycloheptyl, substituted cycloheptyl, bicycloheptyl, bicyclooctyl, bicyclononyl, substituted bicycloalknyl, adamantyl, substituted adamantyl and the like; and
wherein said amount is sufficient to suppress expression of at least one ER/golgi resident protein involved in proliferation-dependent protein trafficking between the ER and golgi, such that the cell proliferation associated with the disease condition is inhibited.

8. A method for selectively inhibiting cytokine responses associated with a disease condition, comprising administering an amount of a composition sufficient to suppress expression of at least one ER/golgi resident protein involved in cytokine-dependent protein trafficking between the ER and golgi, such that the cytokine responses associated with the disease condition are inhibited.

9. The method of claim 8, wherein said composition comprises a compound described in claim 4.

10. A method for selectively inhibiting viral replication, comprising administering an amount of a composition sufficient to suppress expression of at least one ER/golgi resident protein involved in viral protein trafficking between the ER and golgi, such that viral replication is inhibited.

11. The method of claim 10, wherein said composition comprises a compound described in claim 4.

12. A method for selectively reducing B-cell secretion of IgE associated with an allergic reaction, comprising administering an amount of a composition sufficient to suppress expression of at least one ER/golgi resident protein involved in protein trafficking, such that the B-cell secretion of IgE is reduced.

13. The method of claim 12, wherein said composition comprises a compound described in claim 4.

14. A method for diminishing GS28-mediated protein trafficking, comprising administering an amount of a composition sufficient to suppress GS28 expression such that GS28-mediated protein trafficking is diminished.

15. A method for modifying effects of external influences on eukaryotic cells, wherein said external influences depend on GS28-mediated protein trafficking, the method comprising administering an amount of a composition sufficient to alter GS28 expression in the cells such that the external influences are modified.

16. The method of claim 15, wherein said composition comprises a compound described in claim 4.

17. A method for treating a viral infection, comprising administering an amount of a composition sufficient to reduce GS28 expression and thereby reduce progeny virion assembly, such that the viral infection is treated.

18. A method for treating cancer, comprising administering an amount of an agent sufficient to inhibit expression of at least one ER-golgi protein, wherein said at least one ER-golgi protein is required for cancer cell proliferation.
